(19) United States
(12) Patent Application Publication
(10) Pub. No.: US 2024/0376512 A1
(43) Pub. Date: Nov. 14, 2024

(54) OVER EXPRESSION OF FOLDASES AND CHAPERONES IMPROVES PROTEIN PRODUCTION

(71) Applicant: DUPONT NUTRITION BIOSCIENCES APS, Kongens Lyngby (DK)

(72) Inventors: Steven Kim, Fremont, CA (US); Suzanne Lantz, San Carlos, CA (US); Robert Pratt, II, San Jose, CA (US)

(21) Appl. No.: 18/690,504

(22) PCT Filed: Sep. 2, 2022
(86) PCT No.: PCT/US2022/075905

§ 371 Date: Mar. 8, 2024

Related U.S. Application Data
(60) Provisional application No. 63/242,245, filed on Sep. 9, 2021.

(51) Int. Cl. C12P 21/02; C07K 14/37; C12N 1/14; C12N 9/24; C12N 9/50; C12R 1/885

(52) U.S. Cl. CPC C12P 21/02; C07K 14/37; C12N 1/14; C12N 9/2402; C12N 9/50; C07K 2319/036; C12N 2510/00; C12R 2001/885; C12Y 302/01091; C12Y 304/23004

(57) ABSTRACT

The present teachings provide methods for increasing protein secretion, e.g., chymosin in filamentous fungi by coexpressing certain chaperone(s) and/or foldase(s). The present teachings also provide filamentous fungi containing certain chaperone(s) and/or foldase(s) and a protein of interest for increased secretion.

### BACKGROUND OF THE INVENTION

Protein secretion is an important aspect of protein production in various cell expression systems. One of the factors associated with protein secretion is protein folding. Many proteins can be reversibly unfolded and refolded in vitro at dilute concentrations since all of the information required to specify a properly folded protein structure is present in the amino acid sequence of a protein. However, protein folding in vivo occurs in a concentrated milieu of numerous proteins in which intermolecular aggregation reactions compete with the intramolecular folding process. The first step in the eukaryotic secretory pathway is translocation of the nascent polypeptide across the ER membrane. Correct folding and assembly of a polypeptide occurs in the ER within the secretory pathway. Secretion is often the bottleneck in trying to over-express various classes of proteins in diverse expression systems. There is a need in the art to produce proteins efficiently in cellular production systems.

### SUMMARY OF THE INVENTION

In accordance with an aspect of the present invention, it has been discovered that expression hosts may be modified to reduce the presence of secreted enzymatic side activities that could interfere with the use of the expressed, heterologous protein in final (e.g., food) products that contain hydrocolloids and polysaccharides such as guar, cellulose, carboxymethyl cellulose, starch, xylan, and pectin.

In accordance with an aspect of the present invention, an engineered Trichoderma filamentous fungus host cell is presented having an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene. Optionally, the secretion enhancing protein is bip1, ppi1 or sil1. Optionally, said bip1 protein is a polypeptide having an amino acid sequence at least 60%, at least 65%, at least 70%, at least 80%, at least 85%, at least 90%, at least 95%, at least 99% or 100% sequence identity to SEQ ID NO: 16 or a mature version thereof, said ppi1 is a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 is a polypeptide having an amino acid sequence at least 60%, at least 65%, at least 70%, at least 80%, at least 85%, at least 90%, at least 95%, at least 99% or 100% sequence identity to SEQ ID NO:30 or a mature version thereof.

Optionally, the Trichoderma host cell is selected from the group consisting of Trichoderma harzianum, Trichoderma koningii, Trichoderma longibrachiatum, Trichoderma reesei, and Trichoderma viride. Optionally, the Trichoderma host cell is Trichoderma reesei.

In preferred aspects of the present invention, the host cell has a heterologous gene expressing a secretable polypeptide. Optionally, the secretable polypeptide is chymosin.

Optionally, the chymosin is bovine chymosin, camel chymosin, llama chymosin or alpaca chymosin.

Optionally, the bovine chymosin is a polypeptide having an amino acid sequence at least 60%, at least 65%, at least 70%, at least 80%, at least 85%, at least 90%, at least 95%, at least 99% or 100% sequence identity to SEQ ID NO:48, the camel chymosin is a polypeptide having an amino acid sequence at least 60%, at least 65%, at least 70%, at least 80%, at least 85%, at least 90%, at least 95%, at least 99% or 100% sequence identity to SEQ ID NO:49, the llama chymosin is a polypeptide having an amino acid sequence at least 60%, at least 65%, at least 70%, at least 80%, at least 85%, at least 90%, at least 95%, at least 99% or 100% sequence identity to SEQ ID NO:50 and the alpaca chymosin is a polypeptide having an amino acid sequence at least 60%, at least 65%, at least 70%, at least 80%, at least 85%, at least 90%, at least 95%, at least 99% or 100% sequence identity to SEQ ID NO:51.

Optionally, the chymosin is bovine chymosin. Still more preferably, the bovine chymosin comprises a polypeptide having an amino acid sequence according to SEQ ID NO:48.

Optionally, the chymosin is expressed through a Trichoderma reesei promoter. More preferably, the chymosin is expressed under a cbh1 promoter.

Optionally, the chymosin is produced as a fusion protein. Optionally, the chymosin is produced as a fusion protein with a CBHI, or a portion thereof. Optionally, the chymosin is produced as a fusion protein with a CBHI, or a portion thereof, and the CBHI amino acid sequence is altered to reduce or eliminate catalytic activity.

In another aspect of the present invention, use is presented of a host cell as described above for producing chymosin.

In another aspect of the present invention, a method is presented for production of a secretable polypeptide in an engineered Trichoderma filamentous fungus host cell having an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene. Optionally, the secretion enhancing protein is bip1, ppi1 or sil1. Optionally, the bip1 protein is a polypeptide having an amino acid sequence at least 60%, at least 65%, at least 70%, at least 80%, at least 85%, at least 90%, at least 95%, at least 99% or 100% sequence identity to SEQ ID NO:16 or a mature version thereof, said ppi1 is a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 is a polypeptide having an amino acid sequence at least 60%, at least 65%, at least 70%, at least 80%, at least 85%, at least 90%, at least 95%, at least 99% or 100% sequence identity to SEQ ID NO:30 or a mature version thereof.

Optionally, the Trichoderma host cell is selected from the group consisting of Trichoderma harzianum, Trichoderma koningii, Trichoderma longibrachiatum, Trichoderma reesei, and Trichoderma viride. More preferably, the Trichoderma host cell is Trichoderma reesei.

Optionally, the host cell has a heterologous gene expressing a secretable polypeptide. Optionally, the secretable polypeptide is chymosin. Optionally, the chymosin is bovine chymosin, camel chymosin, llama chymosin or alpaca chymosin.

Optionally, the bovine chymosin is a polypeptide having an amino acid sequence at least 60%, at least 65%, at least 70%, at least 80%, at least 85%, at least 90%, at least 95%, at least 99% or 100% sequence identity to SEQ ID NO:48, the camel chymosin is a polypeptide having an amino acid sequence at least 60%, at least 65%, at least 70%, at least 80%, at least 85%, at least 90%, at least 95%, at least 99% or 100% sequence identity to SEQ ID NO:49, the llama chymosin is a polypeptide having an amino acid sequence at least 60%, at least 65%, at least 70%, at least 80%, at least 85%, at least 90%, at least 95%, at least 99% or 100% sequence identity to SEQ ID NO:50 and the alpaca chymosin is a polypeptide having an amino acid sequence at least 60%, at least 65%, at least 70%, at least 80%, at least 85%, at least 90%, at least 95%, at least 99% or 100% sequence identity to SEQ ID NO:51.

Optionally, the chymosin is bovine chymosin. Still more preferably, the bovine chymosin comprises a polypeptide having an amino acid sequence according to SEQ ID NO:48.

Optionally, the chymosin is expressed through a Trichoderma reesei promoter. Optionally, the chymosin is expressed under a cbh1 promoter.

Optionally, the chymosin is produced as a fusion protein. Optionally, the chymosin is produced as a fusion protein with a CBHI, or a portion thereof. Optionally, the chymosin is produced as a fusion protein with a CBHI, or a portion thereof, and the CBHI amino acid sequence is altered to reduce or eliminate catalytic activity.

In the above-described host cell, optionally, the secretion level of the chymosin in the cell is at least 50 mg/liter when the host cell grows in a fermentation condition.

In accordance with an aspect of the present invention, a supernatant is presented from a culture of a host cell as described above wherein the supernatant contains a substantial amount of chymosin, but not a substantial amount of the host cell.

In accordance with an aspect of the present invention, an isolated mutant of a parental Trichoderma strain expressing a heterologous polynucleotide and one or more enzyme genes selected from the group consisting of a 1st enzyme (cbh1) gene encoding a polypeptide having an amino acid sequence having at least 60%, at least 65%, at least 70%, at least 80%, at least 85%, at least 90%, at least 95%, at least 99% or 100% sequence identity to SEQ ID NO:52 or the mature polypeptide thereof, a 2nd enzyme (cbh2) gene encoding a polypeptide having an amino acid sequence having at least 60%, at least 65%, at least 70%, at least 80%, at least 85%, at least 90%, at least 95%, at least 99% or 100% sequence identity to SEQ ID NO:53 or the mature polypeptide thereof, a 3rd enzyme (egl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:54 or the mature polypeptide thereof, a 4th enzyme (egl2) gene encoding a polypeptide having an amino acid sequence having at least 60%, at least 65%, at least 70%, at least 80%, at least 85%, at least 90%, at least 95%, at least 99% or 100% sequence identity to SEQ ID NO:55 or the mature polypeptide thereof, a 5th enzyme (gap1) gene encoding a polypeptide having an amino acid sequence having at least 60%, at least 65%, at least 70%, at least 80%, at least 85%, at least 90%, at least 95%, at least 99% or 100% sequence identity to SEQ ID NO:65 or the mature polypeptide thereof, a 6th enzyme (egl5) gene encoding a polypeptide having an amino acid sequence having at least 60%, at least 65%, at least 70%, at least 80%, at least 85%, at least 90%, at least 95%, at least 99% or 100% sequence identity to SEQ ID NO:58 or the mature polypeptide thereof, a 7th enzyme (egl3) gene encoding a polypeptide having an amino acid sequence having at least 60%, at least 65%, at least 70%, at least 80%, at least 85%, at least 90%, at least 95%, at least 99% or 100% sequence identity to SEQ ID NO:56 or the mature polypeptide thereof, a 8th enzyme (egl4) gene encoding a polypeptide having an amino acid sequence having at least 60%, at least 65%, at least 70%, at least 80%, at least 85%, at least 90%, at least 95%, at least 99% or 100% sequence identity to SEQ ID NO:57 or the mature polypeptide thereof, a 9th enzyme (egl6) gene encoding a polypeptide having an amino acid sequence having at least 60%, at least 65%, at least 70%, at least 80%, at least 85%, at least 90%, at least 95%, at least 99% or 100% sequence identity to SEQ ID NO:59 or the mature polypeptide thereof, a 10th enzyme (man1) gene encoding a polypeptide having an amino acid sequence having at least 60%, at least 65%, at least 70%, at least 80%, at least 85%, at least 90%, at least 95%, at least 99% or 100% sequence identity to SEQ ID NO:60 or the mature polypeptide thereof, a 11th enzyme (xyn2) gene encoding a polypeptide having an amino acid sequence having at least 60%, at least 65%, at least 70%, at least 80%, at least 85%, at least 90%, at least 95%, at least 99% or 100% sequence identity to SEQ ID NO:61 or the mature polypeptide thereof, a 12th enzyme (xyn3) gene encoding a polypeptide having an amino acid sequence having at least 60%, at least 65%, at least 70%, at least 80%, at least 85%, at least 90%, at least 95%, at least 99% or 100% sequence identity to SEQ ID NO:62 or the mature polypeptide thereof; a 13th enzyme (bgl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:63 or the mature polypeptide thereof, a 14th enzyme (tpp1) gene encoding a polypeptide having an amino acid sequence having at least 60%, at least 65%, at least 70%, at least 80%, at least 85%, at least 90%, at least 95%, at least 99% or 100% sequence identity to SEQ ID NO:64 or the mature polypeptide thereof, and a 15th enzyme (sed2) gene encoding a polypeptide having an amino acid sequence having at least 60%, at least 65%, at least 70%, at least 80%, at least 85%, at least 90%, at least 95%, at least 99% or 100% sequence identity to SEQ ID NO:66 or the mature polypeptide thereof, wherein said one or more enzyme genes are modified rendering the mutant strain deficient in their production relative to the parental strain when cultivated under the same conditions.

Optionally, the isolated mutant of a parental Trichoderma strain has a 1^{st} enzyme (cbh1) gene encoding a polypeptide having an amino acid sequence having at least 60%, at least 65%, at least 70%, at least 80%, at least 85%, at least 90%, at least 95%, at least 99% or 100% sequence identity to SEQ ID NO:52 or the mature polypeptide thereof, a 2^{nd} enzyme (cbh2) gene encoding a polypeptide having an amino acid sequence having at least 60%, at least 65%, at least 70%, at least 80%, at least 85%, at least 90%, at least 95%, at least 99% or 100% sequence identity to SEQ ID NO:53 or the mature polypeptide thereof, a 3^{rd} enzyme (egl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:54 or the mature polypeptide thereof, a 4^{th} enzyme (egl2) gene encoding a polypeptide having an amino acid sequence having at least 60%, at least 65%, at least 70%, at least 80%, at least 85%, at least 90%, at least 95%, at least 99% or 100% sequence identity to SEQ ID NO:55 or the mature polypeptide thereof, a 5^{th} enzyme (gap1) gene encoding a polypeptide having an amino acid sequence having at least 60%, at least 65%, at least 70%, at least 80%, at least 85%, at least 90%, at least 95%, at least 99% or 100% sequence identity to SEQ ID NO:65 or the mature polypeptide thereof, a 6^{th} enzyme (egl5) gene encoding a polypeptide having an amino acid sequence having at least 60%, at least 65%, at least 70%, at least 80%, at least 85%, at least 90%, at least 95%, at least 99% or 100% sequence identity to SEQ ID NO:58 or the mature polypeptide thereof, a 7^{th} enzyme (egl3) gene encoding a polypeptide having an amino acid sequence having at least 60%, at least 65%, at least 70%, at least 80%, at least 85%, at least 90%, at least 95%, at least 99% or 100% sequence identity to SEQ ID NO:56 or the mature polypeptide thereof, a 8^{th} enzyme (egl4) gene encoding a polypeptide having an amino acid sequence having at least 60%, at least 65%, at least 70%, at least 80%, at least 85%, at least 90%, at least 95%, at least 99% or 100% sequence identity to SEQ ID NO:57 or the mature polypeptide thereof, a 9^{th} enzyme (egl6) gene encoding a polypeptide having an amino acid sequence having at least 60%, at least 65%, at least 70%, at least 80%, at least 85%, at least 90%, at least 95%, at least 99% or 100% sequence identity to SEQ ID NO:59 or the mature polypeptide thereof, a 10^{th} enzyme (man1) gene encoding a polypeptide having an amino acid sequence having at least 60%, at least 65%, at least 70%, at least 80%, at least 85%, at least 90%, at least 95%, at least 99% or 100% sequence identity to SEQ ID NO:60 or the mature polypeptide thereof, a 11^{th} enzyme (xyn2) gene encoding a polypeptide having an amino acid sequence having at least 60%, at least 65%, at least 70%, at least 80%, at least 85%, at least 90%, at least 95%, at least 99% or 100% sequence identity to SEQ ID NO:61 or the mature polypeptide thereof, a 12^{th} enzyme (xyn3) gene encoding a polypeptide having an amino acid sequence having at least 60%, at least 65%, at least 70%, at least 80%, at least 85%, at least 90%, at least 95%, at least 99% or 100% sequence identity to SEQ ID NO:62 or the mature polypeptide thereof; a 13^{th} enzyme (bgl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:63 or the mature polypeptide thereof, a 14^{th} enzyme (tpp1) gene encoding a polypeptide having an amino acid sequence having at least 60%, at least 65%, at least 70%, at least 80%, at least 85%, at least 90%, at least 95%, at least 99% or 100% sequence identity to SEQ ID NO:64 or the mature polypeptide thereof, and a 15^{th} enzyme (sed2) gene encoding a polypeptide having an amino acid sequence having at least 60%, at least 65%, at least 70%, at least 80%, at least 85%, at least 90%, at least 95%, at least 99% or 100% sequence identity to SEQ ID NO:66 or the mature polypeptide thereof, wherein said enzyme genes are modified rendering the mutant strain deficient in their production relative to the parental strain when cultivated under the same conditions.

Optionally, the isolated mutant of a parental Trichoderma strain has an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene. Optionally, the secretion enhancing protein is bip1, ppi1 or sil1.

Optionally, said bip1 protein is a polypeptide having an amino acid sequence at least 60%, at least 65%, at least 70%, at least 80%, at least 85%, at least 90%, at least 95%, at least 99% or 100% sequence identity to SEQ ID NO:16 or a mature version thereof, said ppi1 is a polypeptide having an amino acid sequence at least 60%, at least 65%, at least 70%, at least 80%, at least 85%, at least 90%, at least 95%, at least 99% or 100% sequence identity to SEQ ID NO: 25 or a mature version thereof and said sil1 is a polypeptide having an amino acid sequence at least 60%, at least 65%, at least 70%, at least 80%, at least 85%, at least 90%, at least 95%, at least 99% or 100% sequence identity to SEQ ID NO:30 or a mature version thereof.

Optionally, the Trichoderma host cell is selected from the group consisting of Trichoderma harzianum, Trichoderma koningii, Trichoderma longibrachiatum, Trichoderma reesei, and Trichoderma viride. Optionally, the Trichoderma host cell is Trichoderma reesei.

In preferred aspects of the present invention, the host cell has a heterologous gene expressing a secretable polypeptide. Optionally, the secretable polypeptide is chymosin. Optionally, the chymosin is bovine chymosin, camel chymosin, llama chymosin or alpaca chymosin.

Optionally, the bovine chymosin is a polypeptide having an amino acid sequence at least 60%, at least 65%, at least 70%, at least 80%, at least 85%, at least 90%, at least 95%, at least 99% or 100% sequence identity to SEQ ID NO:48, the camel chymosin is a polypeptide having an amino acid sequence at least 60%, at least 65%, at least 70%, at least 80%, at least 85%, at least 90%, at least 95%, at least 99% or 100% sequence identity to SEQ ID NO:49, the llama chymosin is a polypeptide having an amino acid sequence at least 60%, at least 65%, at least 70%, at least 80%, at least 85%, at least 90%, at least 95%, at least 99% or 100% sequence identity to SEQ ID NO:50 and the alpaca chymosin is a polypeptide having an amino acid sequence at least 60%, at least 65%, at least 70%, at least 80%, at least 85%, at least 90%, at least 95%, at least 99% or 100% sequence identity to SEQ ID NO:51.

Optionally, the chymosin is bovine chymosin. Optionally, the bovine chymosin comprises a polypeptide having an amino acid sequence according to SEQ ID NO:48.

Optionally, the chymosin is expressed through a Trichoderma reesei promoter. Optionally, the chymosin is expressed under a cbh1 promoter.

Optionally, the chymosin is produced as a fusion protein. Optionally, the chymosin is produced as a fusion protein with a CBHI, or a portion thereof. Optionally, the chymosin is produced as a fusion protein with a CBHI, or a portion thereof, and the CBHI amino acid sequence is altered to reduce or eliminate catalytic activity.

In accordance with an aspect of the present invention, use is presented of an isolated mutant of a parental Trichoderma strain as described above to produce chymosin. Optionally, the isolated mutant of a parental Trichoderma strain further has one or more pectinase genes selected from the group consisting of a 1^{st} pectinase (pec1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:67 or the mature polypeptide thereof and a 2^{nd} pectinase (pec2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:68 or the mature polypeptide thereof, wherein said one or more pectinase genes are modified rendering the mutant strain deficient in their production relative to the parental strain when cultivated under the same conditions.

Optionally, the isolated mutant of a parental Trichoderma reesei strain has a 1^{st} pectinase (pec1) gene encoding a polypeptide having an amino acid sequence having at least 60%, at least 65%, at least 70%, at least 80%, at least 85%, at least 90%, at least 95%, at least 99% or 100% sequence identity to SEQ ID NO:67 or the mature polypeptide thereof and a 2^{nd} pectinase (pec2) gene encoding a polypeptide having an amino acid sequence having at least 60%, at least 65%, at least 70%, at least 80%, at least 85%, at least 90%, at least 95%, at least 99% or 100% sequence identity to SEQ ID NO:68 or the mature polypeptide thereof, wherein said pectinase genes are modified rendering the mutant strain deficient in their production of said 1^{st} and said 2^{nd} pectinase genes relative to the parental strain when cultivated under the same conditions.

Optionally, the isolated mutant of a parental Trichoderma reesei strain further has one or more amylase genes selected from the group consisting of a 1^{st} amylase (Trire2_105956) gene encoding a polypeptide having an amino acid sequence having at least 60%, at least 65%, at least 70%, at least 80%, at least 85%, at least 90%, at least 95%, at least 99% or 100% sequence identity to SEQ ID NO:69 or the mature polypeptide thereof and a 2^{nd} amylase (Trire2_123368) gene encoding a polypeptide having an amino acid sequence having at least 60%, at least 65%, at least 70%, at least 80%, at least 85%, at least 90%, at least 95%, at least 99% or 100% sequence identity to SEQ ID NO:70 or the mature polypeptide thereof, a 3^{rd} amylase (Trire2_1885) gene encoding a polypeptide having an amino acid sequence having at least 60%, at least 65%, at least 70%, at least 80%, at least 85%, at least 90%, at least 95%, at least 99% or 100% sequence identity to SEQ ID NO:71 or the mature polypeptide thereof and a 4^{th} amylase (Trire2_57128) gene encoding a polypeptide having an amino acid sequence having at least 60%, at least 65%, at least 70%, at least 80%, at least 85%, at least 90%, at least 95%, at least 99% or 100% sequence identity to SEQ ID NO:72 or the mature polypeptide thereof, wherein said one or more amylase genes are modified rendering the mutant strain deficient in their production relative to the parental strain when cultivated under the same conditions.

Optionally, the isolated mutant of a parental Trichoderma reesei strain has a 1^{st} amylase (Trire2_105956) gene encoding a polypeptide having an amino acid sequence having at least 60%, at least 65%, at least 70%, at least 80%, at least 85%, at least 90%, at least 95%, at least 99% or 100% sequence identity to SEQ ID NO:69 or the mature polypeptide thereof and a 2^{nd} amylase (Trire2_123368) gene having a polypeptide comprising an amino acid sequence having at least 60%, at least 65%, at least 70%, at least 80%, at least 85%, at least 90%, at least 95%, at least 99% or 100% sequence identity to SEQ ID NO:70 or the mature polypeptide thereof, a 3^{rd} amylase (Trire2_1885) gene encoding a polypeptide having an amino acid sequence having at least 60%, at least 65%, at least 70%, at least 80%, at least 85%, at least 90%, at least 95%, at least 99% or 100% sequence identity to SEQ ID NO:71 or the mature polypeptide thereof or a 4^{th} amylase (Trire2_57128) gene encoding a polypeptide having an amino acid sequence having at least 60%, at least 65%, at least 70%, at least 80%, at least 85%, at least 90%, at least 95%, at least 99% or 100% sequence identity to SEQ ID NO:72 or the mature polypeptide thereof, wherein said amylase genes are modified rendering the mutant strain deficient in their production relative to the parental strain when cultivated under the same conditions.

### BRIEF DESCRIPTION OF THE SEQUENCES

SEQ ID NO:1 is bip1 nucleic acid.

SEQ ID NO:2 is clx1 nucleic acid.

SEQ ID NO:3 is ero1 nucleic acid.

SEQ ID NO:4 is lhs1 nucleic acid.

SEQ ID NO:5 is prp3 nucleic acid.

SEQ ID NO:6 is prp4 nucleic acid.

SEQ ID NO:7 is prp1 nucleic acid.

SEQ ID NO:8 is tig1 nucleic acid.

SEQ ID NO:9 is pdi1 nucleic acid.

SEQ ID NO:10 is ppi1 nucleic acid.

SEQ ID NO:11 is ppi2 nucleic acid.

SEQ ID NO:12 is scj1 nucleic acid.

SEQ ID NO:13 is erv2 nucleic acid.

SEQ ID NO:14 is EDEM nucleic acid.

SEQ ID NO:15 is sil1 nucleic acid.

SEQ ID NO:16 is bip1 protein.

SEQ ID NO:17 is clx1 protein.

SEQ ID NO:18 is ero1 protein.

SEQ ID NO:19 is lhs1 protein.

SEQ ID NO:20 is prp3 protein.

SEQ ID NO:21 prp4 protein.

SEQ ID NO:22 is prp1 protein.

SEQ ID NO:23 is tig1 protein.

SEQ ID NO:24 is pdi1 protein.

SEQ ID NO: 25 is pp1 protein.

SEQ ID NO:26 is ppi2 protein.

SEQ ID NO:27 is scj1 protein.

SEQ ID NO:28 is erv2 protein.

SEQ ID NO:29 is EDEM protein.

SEQ ID NO:30 is sil1 protein.

SEQ ID NO:31 is the F-attB1 PCR primer.

SEQ ID NO:32 is the R-attB2 PCR primer.

SEQ ID NO:33 is the CBH1 forward primer.

SEQ ID NO:34 is the CBH1 reverse primer.

SEQ ID NO:35 is the Bip1 forward primer.

SEQ ID NO:36 is the Bip1 reverse primer.

SEQ ID NO:37 is the Chymosin forward primer.

SEQ ID NO:38 is the Chymosin reverse primer.

SEQ ID NO:39 is the CBHI linker region with SpeI restriction site change.

SEQ ID NO:40 is the hph1 PCR primer.

SEQ ID NO:41 is the hph2 PCR primer.

SEQ ID NO:42 is the synthetic pro-chymosin gene.

SEQ ID NO:43 is the bip1 promoter.

SEQ ID NO:44 is the bovine chymosin nucleic acid.

SEQ ID NO:45 is the camel chymosin nucleic acid.

SEQ ID NO:46 is the llama chymosin nucleic acid.

SEQ ID NO:47 is the alpaca chymosin nucleic acid.

SEQ ID NO:48 is the bovine chymosin protein.

SEQ ID NO:49 is the camel chymosin protein.

SEQ ID NO:50 is the llama chymosin protein.

SEQ ID NO:51 is the alpaca chymosin protein.

SEQ ID NO:52 is the Trichoderma CBH1 protein.

SEQ ID NO:53 is the Trichoderma CBH2 protein.

SEQ ID NO:54 is the Trichoderma EGL1 protein.

SEQ ID NO:55 is the Trichoderma EGL2 protein.

SEQ ID NO:56 is the Trichoderma EGL3 protein.

SEQ ID NO:57 is the Trichoderma EGL4 protein.

SEQ ID NO:58 is the Trichoderma EGL5 protein.

SEQ ID NO:59 is the Trichoderma EGL6 protein.

SEQ ID NO:60 is the Trichoderma MAN1 protein.

SEQ ID NO:61 is the Trichoderma XYN2 protein.

SEQ ID NO:62 is the Trichoderma XYN3 protein.

SEQ ID NO:63 is the Trichoderma BGL1 protein.

SEQ ID NO:64 is the Trichoderma TPP1 protein.

SEQ ID NO:65 is the Trichoderma GAP1 protein.

SEQ ID NO:66 is the Trichoderma SED2 protein.

SEQ ID NO:67 is the Trichoderma PEC1 protein.

SEQ ID NO:68 is the Trichoderma PEC2 protein.

SEQ ID NO:69 is the Trichoderma Trire2_105956 protein.

SEQ ID NO:70 is the Trichoderma Trire2_123368 protein.

SEQ ID NO:71 is the Trichoderma Trire2_1885 protein.

SEQ ID NO:72 is the Trichoderma Trire2_57128 protein.

### DESCRIPTION OF VARIOUS EMBODIMENTS

### Definition Section

The term “promoter” is defined herein as a nucleic acid that directs transcription of a downstream polynucleotide in a cell. In certain cases, the polynucleotide may contain a coding sequence and the promoter may direct the transcription of the coding sequence into translatable RNA.

The term “isolated” as defined herein means a compound, a protein, cell, nucleic acid sequence or amino acid that is removed from at least one component with which it is naturally associated.

The term “% homology” is used interchangeably herein with the term “% identity” herein and refers to the level of nucleic acid or amino acid sequence identity between the nucleic acid sequences, when aligned using a sequence alignment program. For example, as used herein, 80% homology means the same thing as 80% sequence identity. Exemplary levels of sequence identity include, but are not limited to, 80, 85, 90, 95, 98% or more sequence identity to a given sequence.

The term “coding sequence” is defined herein as a nucleic acid that, when placed under the control of appropriate control sequences including a promoter, is transcribed into mRNA which can be translated into a polypeptide. A coding sequence may contain a single open reading frame, or several open reading frames separated by introns, for example. A coding sequence may be cDNA, genomic DNA, synthetic DNA or recombinant DNA, for example. A coding sequence generally starts at a start codon (e.g., ATG) and ends at a stop codon (e.g., UAA, UAG and UGA).

The term “recombinant” refers to a polynucleotide or polypeptide that does not naturally occur in a host cell. A recombinant molecule may contain two or more naturally occurring sequences that are linked together in a way that does not occur naturally.

The term “heterologous” refers to elements that are not normally associated with each other. For example, if a recombinant host cell produces a heterologous protein, that protein is not produced in a wild-type host cell of the same type, a heterologous promoter is a promoter that is not present in nucleic acid that is endogenous to a wild type host cell, and a promoter operably linked to a heterologous coding sequence is a promoter that is operably linked to a coding sequence that it is not usually operably linked to in a wild-type host cell. A “heterologous” nucleic acid construct or sequence has a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

The term “operably linked” refers to an arrangement of elements that allows them to be functionally related. For example, a promoter is operably linked to a coding sequence if it controls the transcription of the sequence, and a signal sequence is operably linked to a protein if the signal sequence directs the protein through the secretion system of a host cell.

The term “nucleic acid” and “polynucleotide” are used interchangeably and encompass DNA, RNA, cDNA, single stranded or double stranded and chemical modifications thereof. Because the genetic code is degenerate, more than one codon may be used to encode a particular amino acid, and the present invention encompasses all polynucleotides, which encode a particular amino acid sequence.

The term “over-expression of a gene” means the introduction of an additional copy or copies of an expression cassette consisting of the gene controlled by either a native or heterologous promoter sequence; or increasing expression of the gene compared to a parent strain by replacing the native promoter with a heterologous promoter in the endogenous gene locus.

The term “DNA construct” as used herein means a nucleic acid sequence that comprises at least two DNA polynucleotide fragments.

The term “signal sequence” refers to a sequence of amino acids at the N-terminal portion of a protein, which facilitates the secretion of the mature form of the protein outside the cell. The mature form of the extracellular protein lacks the signal sequence which is cleaved off during the secretion process.

The term “vector” is defined herein as a polynucleotide designed to carry nucleic acid sequences to be introduced into one or more cell types. Vectors include cloning vectors, expression vectors, shuttle vectors, plasmids, phage or virus particles, DNA constructs, cassettes and the like.

An “expression vector” as used herein means a DNA construct comprising a coding sequence that is operably linked to suitable control sequences capable of effecting expression of a protein in a suitable host. Such control sequences may include a promoter to effect transcription, an optional operator sequence to control transcription, a sequence encoding suitable ribosome binding sites, enhancers and sequences which control termination of transcription and translation. Expression vectors may include regulatory sequences such as promoters, signal sequences, coding sequences and transcription terminators.

As used herein, the terms “polypeptide” and “protein” are used interchangeably and include reference to a polymer of any number of amino acid residues. The terms apply to amino acid polymers in which one or more amino acid residue is an artificial chemical analog of a corresponding naturally occurring amino acid, as well as to naturally occurring amino acid polymers. The terms also apply to polymers containing conservative amino acid substitutions such that the polypeptide remains functional.

A “host” refers to a suitable host for an expression vector comprising a DNA construct encoding a desired protein. A host may be any cell type. The term “filamentous fungi” refers to all filamentous forms of the subdivision Eumycotina (See, Alexopoulos, C. J. (1962), INTRODUCTORY MYCOLOGY, Wiley, New York). These fungi are characterized by a vegetative mycelium with a cell wall composed of chitin, glucans, and other complex polysaccharides. The filamentous fungi of the present teachings are morphologically, physiologically, and genetically distinct from yeasts. Vegetative growth by filamentous fungi is by hyphal elongation and carbon catabolism is obligatory aerobic.

As used herein, “deletion of a gene” or the symbol “A” refer to its removal from the genome of a host cell. Where a gene includes control elements (e.g., enhancer elements) that are not located immediately adjacent to the coding sequence of a gene, deletion of a gene refers to the deletion of the coding sequence, and optionally adjacent promoter and/or terminator sequences.

As used herein, “disruption of a gene” also sometimes called “A” refers broadly to any genetic or chemical manipulation that substantially prevents a cell from producing a functional gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a functional gene product.

The present teachings are based on the discovery that protein secretion in a host can be modulated by a group of chaperones and/or foldases. Accordingly, the present teachings provide methods for increasing protein secretion in a host, e.g., filamentous fungi by coexpressing certain chaperone(s) and/or foldase(s). The present teachings also provide expression hosts, e.g., filamentous fungi containing certain chaperone(s) and/or foldase(s) and a polypeptide of interest for increased secretion.

According to one aspect of the present teachings, it provides methods for increasing the secretion of a polypeptide of interest in a host by expressing a secretion enhancing protein along with the desired polypeptide in the host. The secretion enhancing protein of the present teachings can be any suitable protein associated with protein folding and/or secretion. In some embodiments, the secretion enhancing protein of the present teachings can be a member of a chaperone or a foldase protein family. In some embodiments, the secretion enhancing protein can be a member of a chaperone or a foldase protein family of the host origin. In some embodiments, the secretion enhancing protein includes a combination of a chaperone protein and a foldase protein. In some embodiments, the secretion enhancing protein can be a fragment of a chaperone or foldase protein with substantially the same protein secretion enhancing function as the full-length chaperone or foldase.

In various embodiments, the secretion enhancing protein of the present teachings can be bip1, clx1, ero1, lhs1, prp3, prp4, prp1, tig1, pdi1, ppi1, ppi2, Scj1, erv2, EDEM, and/or sil1 or combinations thereof. In the context of the present teachings, the name of any particular chaperone or foldase means that particular chaperone or foldase from any species, native or recombinant, or any particular chaperone or foldase with an amino acid sequence identical or substantially identical, e.g., at least 50%, 60%, 70%, 80%, 90%, or 95% identical to the corresponding chaperone or foldase sequence illustrated in the present application, or any polypeptide that can be a homolog of that particular chaperone or foldase, e.g., based on function or structure similarities commonly accepted by one skilled in the art. Examples of nucleic acid and polypeptide sequences of bip1, clx1, ero1, lhs1, prp3, prp4, prp1, tig1, pdi1, ppi1, ppi2, Scj1, erv2, EDEM, and sil1 are illustrated in the present application as SEQ ID NOs. 1-30 (see Table 1).

TABLE 1
Exemplary nucleic acid and polypeptide sequences
of secretion enhancing proteins.

| | Exemplary Nucleotide | Exemplary Polypeptide |
|---|---|---|
| Protein | Acid Sequence | Sequence |
| bip1 | SEQ ID NO: 1 | SEQ ID NO: 16 |
| clx1 | SEQ ID NO: 2 | SEQ ID NO: 17 |
| ero1 | SEQ ID NO: 3 | SEQ ID NO: 18 |
| lhs1 | SEQ ID NO: 4 | SEQ ID NO: 19 |
| prp3 | SEQ ID NO: 5 | SEQ ID NO: 20 |
| prp4 | SEQ ID NO: 6 | SEQ ID NO: 21 |
| prp1 | SEQ ID NO: 7 | SEQ ID NO: 22 |
| tig1 | SEQ ID NO: 8 | SEQ ID NO: 23 |
| pdi1 | SEQ ID NO: 9 | SEQ ID NO: 24 |
| ppi1 | SEQ ID NO: 10 | SEQ ID NO: 25 |
| ppi2 | SEQ ID NO: 11 | SEQ ID NO: 26 |
| scj1 | SEQ ID NO: 12 | SEQ ID NO: 27 |
| erv2 | SEQ ID NO: 13 | SEQ ID NO: 28 |
| EDEM | SEQ ID NO: 14 | SEQ ID NO: 29 |
| sil1 | SEQ ID NO: 15 | SEQ ID NO: 30 |

In general, the secretion enhancing protein of the present teachings can be co-expressed along with one or more desired polypeptides, e.g., polypeptides of interest in a host. The expression of the secretion enhancing protein can be under any suitable promoter known or later discovered in the art. In some embodiments, the secretion enhancing protein can be expressed under a promoter native to the host. In some embodiments, the secretion enhancing protein can be expressed under a heterologous promoter. In some embodiments, the secretion enhancing protein can be expressed under a constitutive or inducible promoter.

As used herein, the term “promoter” refers to a nucleic acid sequence that functions to direct transcription of a downstream gene. A promoter can include necessary nucleic acid sequences near the start site of transcription, such as, in the case of a polymerase II type promoter, a TATA element. The promoter together with other transcriptional and translational regulatory nucleic acid sequences, collectively referred to as regulatory sequences controls the expression of a gene. In general, the regulatory sequences include, but are not limited to, promoter sequences, ribosomal binding sites, transcriptional start and stop sequences, translational start and stop sequences, and enhancer or activator sequences. The regulatory sequences will generally be appropriate to and recognized by the host in which the downstream gene is being expressed.

A constitutive promoter is a promoter that is active under most environmental and developmental conditions. An inducible or repressible promoter is a promoter that is active under environmental or developmental regulation. Promoters can be inducible or repressible by changes in environment factors such as, but not limited to, carbon, nitrogen or other nutrient availability, temperature, pH, osmolarity, the presence of heavy metal, the concentration of an inhibitor, stress, or a combination of the foregoing, as is known in the art. Promoters can be inducible or repressible by metabolic factors, such as the level of certain carbon sources, the level of certain energy sources, the level of certain catabolites, or a combination of the foregoing, as is known in the art.

Suitable non-limiting examples of promoters include cbh1, cbh2, egl1, egl2, egl3, egl4, egl5, xyn1, and xyn2, repressible acid phosphatase gene (phoA) promoter of P. chrysogenum (see Graessle et al., Applied and Environmental Microbiology (1997), 63 (2), 753-756), glucose-repressible PCK1 promoter (see Leuker et al. Gene (1997), 192 (2), 235-240), maltose-inducible, glucose-repressible MRP1 promoter (see Munro et al. Molecular Microbiology (2001), 39 (5), 1414-1426), methionine-repressible MET3 promoter (see Liu et al. Eukaryotic Cell (2006), 5 (4), 638-649).

In some embodiments of the present teachings, the promoter in the reporter gene construct is a temperature-sensitive promoter. Preferably, the activity of the temperature-sensitive promoter is repressed by elevated temperature. In some embodiments, the promoter is a catabolite-repressed promoter. In some embodiments, the promoter is repressed by changes in osmolarity. In some embodiments, the promoter is inducible or repressible by the levels of polysaccharides, disaccharides, or monosaccharides.

An example of an inducible promoter useful in the present teachings is the cbh1 promoter of Trichoderma reesei, the nucleotide sequence of which is deposited in GenBank under Accession Number D86235. Other exemplary promoters are promoters involved in the regulation of genes encoding cellulase and hemicellulase enzymes, such as, but not limited to, cbh2, egl1, egl2, egl3, egl5, xyn1 and xyn2.

According to the present teachings, the secretion enhancing protein can be used to increase the secretion of any suitable polypeptide in a host. In some embodiments, the polypeptide can be a heterologous polypeptide. In some embodiments, the polypeptide can be a secretable polypeptide. For example, a secretable polypeptide can be a protein or polypeptide usually secreted outside of a cell or a protein or polypeptide operably linked to a signal sequence, e.g., an amino acid sequence tag leading proteins or polypeptides through the secretion pathway of a cell. Usually, any suitable signal sequence known or later discovered can be used including, without any limitation, signal sequences derived from preprochymosin, e.g., bovine preprochymosin, glucoamylase, e.g., A. niger glucoamylase, aspartic protease, e.g., Rhizomucor miehei or Trichoderma reesei aspartic proteases or cellulases, e.g., Trichoderma reesei cellobiohydrolase I, cellobiohydrolase II, endoglucanase I, endoglucanase II or endoglucanase III.

In some embodiments, the polypeptide of interest can be a member of the aspartic proteinase family, e.g., family A1 of aspartic proteinases according to the MEROPS classification (Rawlings et al., Nucleic Acids Res (2006) 34: D270-72). This protein family contains endopeptidases with a catalytic center formed by two aspartic acid residues that are active at acidic pH. Chymosins (peptidase 3.4.23.4 by the NC-IUMB classification) are aspartic proteases that perform limited digestion of kappa-casein in neonatal gastric digestion. Bovine chymosin is used to clot milk during cheese making. In some embodiments, the polypeptide of interest can be a member of chymosin family, e.g., chymosin of any species including, without any limitation, chymosin of bovine, sheep, or goat origin. In some embodiments, the polypeptide of interest can be a modified chymosin, e.g., chymosin modified, such as mutated, to increase its function in any cheese making or milk coagulation process or optimize its expression in expression hosts. In some embodiments, the polypeptide of interest can be a fusion chymosin including at least two chymosins from two different species. In the context of the present application, the term “chymosin” means chymosin of any species, native or recombinant, or any polypeptide with substantially the same amino acid sequence as chymosin, e.g., any polypeptide having at least 60%, 70%, 80%, 90%, or 95% sequence identity of a chymosin, or any polypeptide with substantially the same protein folding characteristics of a chymosin, or a chymosin homolog, e.g., based on function or structure similarities commonly accepted by one skilled in the art. In some embodiments, the heterologous protein can be any protein expressible in a filamentous fungal host. Examples of proteins expressible in filamentous fungal hosts include, but are not limited to, laccases, endopeptidases, glucoamylases, alpha-amylase, granular starch hydrolyzing enzyme, cellulases, lipases, xylanases, cutinases, hemicellulases, proteases, oxidases, and combinations thereof. In general, the expression of a desired polypeptide in the present teachings can be under any suitable promoter known or later discovered in the art. In some embodiments, the polypeptide of interest in the present teachings can be expressed under a promoter native to the host. In some embodiments, the polypeptide of interest in the present teachings can be expressed under a heterologous promoter. In some embodiments, the polypeptide of interest in the present teachings can be expressed under a constitutive or inducible promoter. In some embodiments, the polypeptide of interest in the present teachings can be expressed in a Trichoderma expression system with a cellulase promoter, e.g., cbh1 promoter.

According to the present teachings, the secretion enhancing protein can be used in any host, e.g., expression host to increase the secretion of a desired polypeptide in the host. For example, the expression hosts of the present teachings can be filamentous fungi. In general, the filamentous fungi of the present teachings are eukaryotic microorganisms and include all filamentous forms of the subdivision Eumycotina. These fungi are characterized by a vegetative mycelium with a cell wall composed of chitin, beta-glucan, and other complex polysaccharides. In various embodiments, the filamentous fungi of the present teachings are morphologically, physiologically, and genetically distinct from yeasts. In some embodiments, the filamentous fungi of the present teachings include, but are not limited to the following genera: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus. In some embodiments, the filamentous fungi of the present teachings include, but are not limited to the following: A. nidulans, A. niger, A. awamori, e.g., NRRL 3112, ATCC 22342 (NRRL 3112), ATCC 44733, ATCC 14331 and strain UVK 143f, A. oryzae, e.g., ATCC 11490, N. crassa, Trichoderma reesei, e.g., NRRL 15709, ATCC 13631, 56764, 56765, 56766, 56767, and Trichoderma viride, e.g., ATCC 32098 and 32086.

According to another aspect of the present teachings, it provides an expression host expressing a secretion enhancing protein and a desired polypeptide, e.g., polypeptide of interest.

In some embodiments, the expression host of the present teachings contains a first polynucleotide encoding a secretion enhancing protein and a second polynucleotide encoding a polypeptide of interest. In some embodiments, the expression host of the present teachings contains a first polynucleotide encoding a secretion enhancing protein, a second polynucleotide encoding a polypeptide of interest, and a third polynucleotide encoding a secretion enhancing protein, e.g., different from the one encoded by the first polynucleotide.

In some embodiments, the expression host of the present teachings contains a first polynucleotide encoding a secretion enhancing protein that can be a chaperone or foldase protein and a second polynucleotide encoding a polypeptide of interest. In some embodiments, the expression host of the present teachings contains a first polynucleotide encoding a secretion enhancing protein that can be a chaperone, a second polynucleotide encoding a polypeptide of interest, and a third polynucleotide encoding a secretion enhancing protein that can be a foldase.

According to the present teachings, the first, second, and/or third polynucleotide in the expression host of the present teachings can be operably linked to one or more promoters, e.g., native or heterologous promoters of the expression host. Any suitable promoter can be used in the present teachings. In some embodiments, the promoter operably linked to the first and/or third polynucleotide can be a constitutive or inducible promoter. In some embodiments, the promoter operably linked to the second polynucleotide can be a promoter native to the expression host containing the second polynucleotide. In some embodiments, the promoter operably linked to the second polynucleotide can be a native promoter associated with any gene characteristic of active transcription or expression in the expression host. In some embodiments, the promoter operably linked to the second polynucleotide can be a modified native promoter, e.g., mutated native promoter with enhanced transcription activity of the promoter. In some embodiments, the promoter operably linked to the second polypeptide in a Trichoderma expression system can be a cellulase promoter, e.g., cbh1 promoter.

In some embodiments the desired polypeptide may be produced as a fusion polypeptide. In some embodiments the desired polypeptide may be fused to a polypeptide that is efficiently secreted by a filamentous fungus. In some embodiments the desired polypeptide may be fused to a CBHI polypeptide, or portion thereof. In some embodiments the desired polypeptide may be fused to a CBHI polypeptide, or portion thereof, that is altered to minimize or eliminate catalytic activity. In some embodiments the desired polypeptide may be fused to a polypeptide to enhance secretion, facilitate subsequent purification or enhance stability.

In general, the first, second, and/or third polynucleotide in the expression host of the present teachings can be either genetically inserted or integrated into the genomic makeup of the expression host, e.g., integrated into the chromosome of the expression host, or existing extrachromosomally, e.g., existing as a replicating vector within the expression host under selection condition for a selection marker carried by the vector.

According to the present teachings, the secretion level of a desired polypeptide in the expression host of the present teachings can be determined by various factors, e.g., growth conditions of the host, etc., however normally higher than the secretion level of the desired polypeptide expressed in the host without the expression of a secretion enhancing protein. In some embodiments, the secretion level of a desired polypeptide, e.g., bovine chymosin in the expression host of the present teachings, e.g., T. reesei can be at least 1 mg/liter, 2 mg/liter, 3 mg/liter, 4 mg/liter, or 5 mg/liter when the host grows in a batch fermentation mode in a shake flask, or at least 50 mg/liter, 100 mg/liter, 150 mg/liter, 200 mg/liter, 250 mg/liter, or 300 mg/liter when the host grows in a fermenter environment with controlled pH, feed-rate, etc. e.g., fed-batch fermentation.

According to yet another aspect of the present teachings, it provides extracts, e.g., solids or supernatant obtained from the culture of the expression host of the present teachings. In some embodiments, the supernatant does not contain substantial amount of the expression host, in some embodiments, the supernatant does not contain any amount of the expression host.

### PREFERRED EMBODIMENTS

In accordance with an aspect of the present invention, it has been discovered that expression hosts may be modified to reduce the presence of secreted enzymatic side activities that could interfere with the use of the expressed, heterologous protein in final (e.g., food) products that contain hydrocolloids and polysaccharides such as guar, cellulose, carboxymethyl cellulose, starch, xylan, and pectin.

In accordance with an aspect of the present invention, an engineered Trichoderma filamentous fungus host cell is presented having an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene. Preferably, the secretion enhancing protein is bip1, ppi1 or sil1. Preferably, said bip1 protein is a polypeptide having an amino acid sequence at least 60%, at least 65%, at least 70%, at least 80%, at least 85%, at least 90%, at least 95%, at least 99% or 100% sequence identity to SEQ ID NO: 16 or a mature version thereof, said ppi1 is a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 is a polypeptide having an amino acid sequence at least 60%, at least 65%, at least 70%, at least 80%, at least 85%, at least 90%, at least 95%, at least 99% or 100% sequence identity to SEQ ID NO:30 or a mature version thereof.

Preferably, the Trichoderma host cell is selected from the group consisting of Trichoderma harzianum, Trichoderma koningii, Trichoderma longibrachiatum, Trichoderma reesei, and Trichoderma viride. More preferably, the Trichoderma host cell is Trichoderma reesei.

In preferred aspects of the present invention, the host cell has a heterologous gene expressing a secretable polypeptide. Preferably, the secretable polypeptide is chymosin. Preferably, the chymosin is bovine chymosin, camel chymosin, llama chymosin or alpaca chymosin.

Preferably, the bovine chymosin is a polypeptide having an amino acid sequence at least 60%, at least 65%, at least 70%, at least 80%, at least 85%, at least 90%, at least 95%, at least 99% or 100% sequence identity to SEQ ID NO:48, the camel chymosin is a polypeptide having an amino acid sequence at least 60%, at least 65%, at least 70%, at least 80%, at least 85%, at least 90%, at least 95%, at least 99% or 100% sequence identity to SEQ ID NO:49, the llama chymosin is a polypeptide having an amino acid sequence at least 60%, at least 65%, at least 70%, at least 80%, at least 85%, at least 90%, at least 95%, at least 99% or 100% sequence identity to SEQ ID NO:50 and the alpaca chymosin is a polypeptide having an amino acid sequence at least 60%, at least 65%, at least 70%, at least 80%, at least 85%, at least 90%, at least 95%, at least 99% or 100% sequence identity to SEQ ID NO:51.

More preferably, the chymosin is bovine chymosin. Still more preferably, the bovine chymosin comprises a polypeptide having an amino acid sequence according to SEQ ID NO:48.

Preferably, the chymosin is expressed through a Trichoderma reesei promoter. More preferably, the chymosin is expressed under a cbh1 promoter.

Preferably, the chymosin is produced as a fusion protein. More preferably, the chymosin is produced as a fusion protein with a CBHI, or a portion thereof. Still more preferably, the chymosin is produced as a fusion protein with a CBHI, or a portion thereof, and the CBHI amino acid sequence is altered to reduce or eliminate catalytic activity.

In another aspect of the present invention, use is presented of a host cell as described above for producing chymosin.

In another aspect of the present invention, a method is presented for production of a secretable polypeptide in an engineered Trichoderma filamentous fungus host cell having an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene. Preferably, the secretion enhancing protein is bip1, ppi1 or sil1. Preferably, said bip1 protein is a polypeptide having an amino acid sequence at least 60%, at least 65%, at least 70%, at least 80%, at least 85%, at least 90%, at least 95%, at least 99% or 100% sequence identity to SEQ ID NO:16 or a mature version thereof, said ppi1 is a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 is a polypeptide having an amino acid sequence at least 60%, at least 65%, at least 70%, at least 80%, at least 85%, at least 90%, at least 95%, at least 99% or 100% sequence identity to SEQ ID NO:30 or a mature version thereof.

Preferably, the Trichoderma host cell is selected from the group consisting of Trichoderma harzianum, Trichoderma koningii, Trichoderma longibrachiatum, Trichoderma reesei, and Trichoderma viride. More preferably, the Trichoderma host cell is Trichoderma reesei.

In preferred aspects of the present invention, the host cell has a heterologous gene expressing a secretable polypeptide. Preferably, the secretable polypeptide is chymosin. Preferably, the chymosin is bovine chymosin, camel chymosin, llama chymosin or alpaca chymosin.

Preferably, the bovine chymosin is a polypeptide having an amino acid sequence at least 60%, at least 65%, at least 70%, at least 80%, at least 85%, at least 90%, at least 95%, at least 99% or 100% sequence identity to SEQ ID NO:48, the camel chymosin is a polypeptide having an amino acid sequence at least 60%, at least 65%, at least 70%, at least 80%, at least 85%, at least 90%, at least 95%, at least 99% or 100% sequence identity to SEQ ID NO:49, the llama chymosin is a polypeptide having an amino acid sequence at least 60%, at least 65%, at least 70%, at least 80%, at least 85%, at least 90%, at least 95%, at least 99% or 100% sequence identity to SEQ ID NO:50 and the alpaca chymosin is a polypeptide having an amino acid sequence at least 60%, at least 65%, at least 70%, at least 80%, at least 85%, at least 90%, at least 95%, at least 99% or 100% sequence identity to SEQ ID NO:51.

More preferably, the chymosin is bovine chymosin. Still more preferably, the bovine chymosin comprises a polypeptide having an amino acid sequence according to SEQ ID NO:48.

Preferably, the chymosin is expressed through a Trichoderma reesei promoter. More preferably, the chymosin is expressed under a cbh1 promoter. Preferably, the chymosin is produced as a fusion protein. More preferably, the chymosin is produced as a fusion protein with a CBHI, or a portion thereof. Still more preferably, the chymosin is produced as a fusion protein with a CBHI, or a portion thereof, and the CBHI amino acid sequence is altered to reduce or eliminate catalytic activity.

In the above-described host cell, it is preferred that the secretion level of the chymosin in the cell is at least 50 mg/liter when the host cell grows in a fermentation condition.

In accordance with an aspect of the present invention, a supernatant is presented from a culture of a host cell as described above wherein the supernatant contains substantial amount of chymosin, but not a substantial amount of the host cell.

In accordance with an aspect of the present invention, it has been discovered that fermentation produced chymosin (sometimes referred to herein as “FPC”) (i.e. chymosin expressed by, for example, filamentous fungi or yeast as opposed to chymosin that is extracted from the fourth stomach of a suckling calf) can have enzymatic side activities leading to off-flavors, poor texture, or interference with other ingredients used in the food products containing cheese or whey. In an aspect of the present invention, it has been discovered that as an alternative to costly purification of FPC (for example by column chromatography), certain host genes may be deleted or inactivated to reduce or eliminate unwanted side activities.

In accordance with an aspect of the present invention, an isolated mutant of a parental Trichoderma strain expressing a heterologous polynucleotide and one or more enzyme genes selected from the group consisting of a 1^{st} enzyme (cbh1) gene encoding a polypeptide having an amino acid sequence having at least 60%, at least 65%, at least 70%, at least 80%, at least 85%, at least 90%, at least 95%, at least 99% or 100% sequence identity to SEQ ID NO:52 or the mature polypeptide thereof, a 2^{nd} enzyme (cbh2) gene encoding a polypeptide having an amino acid sequence having at least 60%, at least 65%, at least 70%, at least 80%, at least 85%, at least 90%, at least 95%, at least 99% or 100% sequence identity to SEQ ID NO:53 or the mature polypeptide thereof, a 3^{rd} enzyme (egl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:54 or the mature polypeptide thereof, a 4^{th} enzyme (egl2) gene encoding a polypeptide having an amino acid sequence having at least 60%, at least 65%, at least 70%, at least 80%, at least 85%, at least 90%, at least 95%, at least 99% or 100% sequence identity to SEQ ID NO:55 or the mature polypeptide thereof, a 5^{th} enzyme (gap1) gene encoding a polypeptide having an amino acid sequence having at least 60%, at least 65%, at least 70%, at least 80%, at least 85%, at least 90%, at least 95%, at least 99% or 100% sequence identity to SEQ ID NO:65 or the mature polypeptide thereof, a 6^{th} enzyme (egl5) gene encoding a polypeptide having an amino acid sequence having at least 60%, at least 65%, at least 70%, at least 80%, at least 85%, at least 90%, at least 95%, at least 99% or 100% sequence identity to SEQ ID NO:58 or the mature polypeptide thereof, a 7^{th} enzyme (egl3) gene encoding a polypeptide having an amino acid sequence having at least 60%, at least 65%, at least 70%, at least 80%, at least 85%, at least 90%, at least 95%, at least 99% or 100% sequence identity to SEQ ID NO:56 or the mature polypeptide thereof, a 8^{th} enzyme (egl4) gene encoding a polypeptide having an amino acid sequence having at least 60%, at least 65%, at least 70%, at least 80%, at least 85%, at least 90%, at least 95%, at least 99% or 100% sequence identity to SEQ ID NO:57 or the mature polypeptide thereof, a 9^{th} enzyme (egl6) gene encoding a polypeptide having an amino acid sequence having at least 60%, at least 65%, at least 70%, at least 80%, at least 85%, at least 90%, at least 95%, at least 99% or 100% sequence identity to SEQ ID NO:59 or the mature polypeptide thereof, a 10^{th} enzyme (man1) gene encoding a polypeptide having an amino acid sequence having at least 60%, at least 65%, at least 70%, at least 80%, at least 85%, at least 90%, at least 95%, at least 99% or 100% sequence identity to SEQ ID NO:60 or the mature polypeptide thereof, a 11^{th} enzyme (xyn2) gene encoding a polypeptide having an amino acid sequence having at least 60%, at least 65%, at least 70%, at least 80%, at least 85%, at least 90%, at least 95%, at least 99% or 100% sequence identity to SEQ ID NO:61 or the mature polypeptide thereof, a 12^{th} enzyme (xyn3) gene encoding a polypeptide having an amino acid sequence having at least 60%, at least 65%, at least 70%, at least 80%, at least 85%, at least 90%, at least 95%, at least 99% or 100% sequence identity to SEQ ID NO:62 or the mature polypeptide thereof; a 13^{th} enzyme (bgl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:63 or the mature polypeptide thereof, a 14^{th} enzyme (tpp1) gene encoding a polypeptide having an amino acid sequence having at least 60%, at least 65%, at least 70%, at least 80%, at least 85%, at least 90%, at least 95%, at least 99% or 100% sequence identity to SEQ ID NO:64 or the mature polypeptide thereof, and a 15^{th} enzyme (sed2) gene encoding a polypeptide having an amino acid sequence having at least 60%, at least 65%, at least 70%, at least 80%, at least 85%, at least 90%, at least 95%, at least 99% or 100% sequence identity to SEQ ID NO:66 or the mature polypeptide thereof, wherein said one or more enzyme genes are modified rendering the mutant strain deficient in their production relative to the parental strain when cultivated under the same conditions.

Preferably, the isolated mutant of a parental Trichoderma strain has a 1^{st} enzyme (cbh1) gene encoding a polypeptide having an amino acid sequence having at least 60%, at least 65%, at least 70%, at least 80%, at least 85%, at least 90%, at least 95%, at least 99% or 100% sequence identity to SEQ ID NO:52 or the mature polypeptide thereof, a 2^{nd} enzyme (cbh2) gene encoding a polypeptide having an amino acid sequence having at least 60%, at least 65%, at least 70%, at least 80%, at least 85%, at least 90%, at least 95%, at least 99% or 100% sequence identity to SEQ ID NO:53 or the mature polypeptide thereof, a 3^{rd} enzyme (egl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:54 or the mature polypeptide thereof, a 4^{th} enzyme (egl2) gene encoding a polypeptide having an amino acid sequence having at least 60%, at least 65%, at least 70%, at least 80%, at least 85%, at least 90%, at least 95%, at least 99% or 100% sequence identity to SEQ ID NO:55 or the mature polypeptide thereof, a 5^{th} enzyme (gap1) gene encoding a polypeptide having an amino acid sequence having at least 60%, at least 65%, at least 70%, at least 80%, at least 85%, at least 90%, at least 95%, at least 99% or 100% sequence identity to SEQ ID NO:65 or the mature polypeptide thereof, a 6^{th} enzyme (egl5) gene encoding a polypeptide having an amino acid sequence having at least 60%, at least 65%, at least 70%, at least 80%, at least 85%, at least 90%, at least 95%, at least 99% or 100% sequence identity to SEQ ID NO:58 or the mature polypeptide thereof, a 7^{th} enzyme (egl3) gene encoding a polypeptide having an amino acid sequence having at least 60%, at least 65%, at least 70%, at least 80%, at least 85%, at least 90%, at least 95%, at least 99% or 100% sequence identity to SEQ ID NO:56 or the mature polypeptide thereof, a 8^{th} enzyme (egl4) gene encoding a polypeptide having an amino acid sequence having at least 60%, at least 65%, at least 70%, at least 80%, at least 85%, at least 90%, at least 95%, at least 99% or 100% sequence identity to SEQ ID NO:57 or the mature polypeptide thereof, a 9^{th} enzyme (egl6) gene encoding a polypeptide having an amino acid sequence having at least 60%, at least 65%, at least 70%, at least 80%, at least 85%, at least 90%, at least 95%, at least 99% or 100% sequence identity to SEQ ID NO:59 or the mature polypeptide thereof, a 10^{th} enzyme (man1) gene encoding a polypeptide having an amino acid sequence having at least 60%, at least 65%, at least 70%, at least 80%, at least 85%, at least 90%, at least 95%, at least 99% or 100% sequence identity to SEQ ID NO:60 or the mature polypeptide thereof, a 11^{th} enzyme (xyn2) gene encoding a polypeptide having an amino acid sequence having at least 60%, at least 65%, at least 70%, at least 80%, at least 85%, at least 90%, at least 95%, at least 99% or 100% sequence identity to SEQ ID NO:61 or the mature polypeptide thereof, a 12^{th} enzyme (xyn3) gene encoding a polypeptide having an amino acid sequence having at least 60%, at least 65%, at least 70%, at least 80%, at least 85%, at least 90%, at least 95%, at least 99% or 100% sequence identity to SEQ ID NO:62 or the mature polypeptide thereof; a 13^{th} enzyme (bgl1) gene encoding a polypeptide having an amino acid sequence having at least 60%, at least 65%, at least 70%, at least 80%, at least 85%, at least 90%, at least 95%, at least 99% or 100% sequence identity to SEQ ID NO:63 or the mature polypeptide thereof, a 14^{th} enzyme (tpp1) gene encoding a polypeptide having an amino acid sequence having at least 60%, at least 65%, at least 70%, at least 80%, at least 85%, at least 90%, at least 95%, at least 99% or 100% sequence identity to SEQ ID NO:64 or the mature polypeptide thereof, and a 15^{th} enzyme (sed2) gene encoding a polypeptide having an amino acid sequence having at least 60%, at least 65%, at least 70%, at least 80%, at least 85%, at least 90%, at least 95%, at least 99% or 100% sequence identity to SEQ ID NO:66 or the mature polypeptide thereof, wherein said enzyme genes are modified rendering the mutant strain deficient in their production relative to the parental strain when cultivated under the same conditions.

Preferably, the isolated mutant of a parental Trichoderma strain has an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene. Preferably, the secretion enhancing protein is bip1, ppi1 or sil1. Preferably, said bip1 protein is a polypeptide having an amino acid sequence at least 60%, at least 65%, at least 70%, at least 80%, at least 85%, at least 90%, at least 95%, at least 99% or 100% sequence identity to SEQ ID NO: 16 or a mature version thereof, said ppi1 is a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 is a polypeptide having an amino acid sequence at least 60%, at least 65%, at least 70%, at least 80%, at least 85%, at least 90%, at least 95%, at least 99% or 100% sequence identity to SEQ ID NO: 30 or a mature version thereof.

Preferably, the Trichoderma host cell is selected from the group consisting of Trichoderma harzianum, Trichoderma koningii, Trichoderma longibrachiatum, Trichoderma reesei, and Trichoderma viride. More preferably, the Trichoderma host cell is Trichoderma reesei.

In preferred aspects of the present invention, the host cell has a heterologous gene expressing a secretable polypeptide. Preferably, the secretable polypeptide is chymosin. Preferably, the chymosin is bovine chymosin, camel chymosin, llama chymosin or alpaca chymosin.

Preferably, the bovine chymosin is a polypeptide having an amino acid sequence at least 60%, at least 65%, at least 70%, at least 80%, at least 85%, at least 90%, at least 95%, at least 99% or 100% sequence identity to SEQ ID NO:48, the camel chymosin is a polypeptide having an amino acid sequence at least 60%, at least 65%, at least 70%, at least 80%, at least 85%, at least 90%, at least 95%, at least 99% or 100% sequence identity to SEQ ID NO:49, the llama chymosin is a polypeptide having an amino acid sequence at least 60%, at least 65%, at least 70%, at least 80%, at least 85%, at least 90%, at least 95%, at least 99% or 100% sequence identity to SEQ ID NO:50 and the alpaca chymosin is a polypeptide having an amino acid sequence at least 60%, at least 65%, at least 70%, at least 80%, at least 85%, at least 90%, at least 95%, at least 99% or 100% sequence identity to SEQ ID NO:51.

More preferably, the chymosin is bovine chymosin. Still more preferably, the bovine chymosin comprises a polypeptide having an amino acid sequence according to SEQ ID NO:48.

Preferably, the chymosin is expressed through a Trichoderma reesei promoter. More preferably, the chymosin is expressed under a cbh1 promoter. Preferably, the chymosin is produced as a fusion protein. More preferably, the chymosin is produced as a fusion protein with a CBHI, or a portion thereof. Still more preferably, the chymosin is produced as a fusion protein with a CBHI, or a portion thereof, and the CBHI amino acid sequence is altered to reduce or eliminate catalytic activity.

In accordance with an aspect of the present invention, use is presented of an isolated mutant of a parental Trichoderma strain as described above to produce chymosin.

Preferably, the isolated mutant of a parental Trichoderma strain further has one or more pectinase genes selected from the group consisting of a 1^{st} pectinase (pec1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:67 or the mature polypeptide thereof and a 2^{nd} pectinase (pec2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:68 or the mature polypeptide thereof, wherein said one or more pectinase genes are modified rendering the mutant strain deficient in their production relative to the parental strain when cultivated under the same conditions.

More preferably, the isolated mutant of a parental Trichoderma reesei strain has a 1^{st} pectinase (pec1) gene encoding a polypeptide having an amino acid sequence having at least 60%, at least 65%, at least 70%, at least 80%, at least 85%, at least 90%, at least 95%, at least 99% or 100% sequence identity to SEQ ID NO:67 or the mature polypeptide thereof and a 2^{nd} pectinase (pec2) gene encoding a polypeptide having an amino acid sequence having at least 60%, at least 65%, at least 70%, at least 80%, at least 85%, at least 90%, at least 95%, at least 99% or 100% sequence identity to SEQ ID NO:68 or the mature polypeptide thereof, wherein said pectinase genes are modified rendering the mutant strain deficient in their production of said 1^{st} and said 2^{nd} pectinase genes relative to the parental strain when cultivated under the same conditions.

Preferably, the isolated mutant of a parental Trichoderma reesei strain further has one or more amylase genes selected from the group consisting of a 1^{st} amylase (Trire2_105956) gene encoding a polypeptide having an amino acid sequence having at least 60%, at least 65%, at least 70%, at least 80%, at least 85%, at least 90%, at least 95%, at least 99% or 100% sequence identity to SEQ ID NO:69 or the mature polypeptide thereof and a 2^{nd} amylase (Trire2_123368) gene encoding a polypeptide having an amino acid sequence having at least 60%, at least 65%, at least 70%, at least 80%, at least 85%, at least 90%, at least 95%, at least 99% or 100% sequence identity to SEQ ID NO:70 or the mature polypeptide thereof, a 3^{rd} amylase (Trire2_1885) gene encoding a polypeptide having an amino acid sequence having at least 60%, at least 65%, at least 70%, at least 80%, at least 85%, at least 90%, at least 95%, at least 99% or 100% sequence identity to SEQ ID NO:71 or the mature polypeptide thereof and a 4^{th} amylase (Trire2_57128) gene encoding a polypeptide having an amino acid sequence having at least 60%, at least 65%, at least 70%, at least 80%, at least 85%, at least 90%, at least 95%, at least 99% or 100% sequence identity to SEQ ID NO:72 or the mature polypeptide thereof, wherein said one or more amylase genes are modified rendering the mutant strain deficient in their production relative to the parental strain when cultivated under the same conditions.

More preferably, the isolated mutant of a parental Trichoderma reesei strain has a 1^{st} amylase (Trire2_105956) gene encoding a polypeptide having an amino acid sequence having at least 60%, at least 65%, at least 70%, at least 80%, at least 85%, at least 90%, at least 95%, at least 99% or 100% sequence identity to SEQ ID NO:69 or the mature polypeptide thereof and a 2^{nd} amylase (Trire2_123368) gene having a polypeptide comprising an amino acid sequence having at least 60%, at least 65%, at least 70%, at least 80%, at least 85%, at least 90%, at least 95%, at least 99% or 100% sequence identity to SEQ ID NO:70 or the mature polypeptide thereof, a 3^{rd} amylase (Trire2_1885) gene encoding a polypeptide having an amino acid sequence having at least 60%, at least 65%, at least 70%, at least 80%, at least 85%, at least 90%, at least 95%, at least 99% or 100% sequence identity to SEQ ID NO:71 or the mature polypeptide thereof or a 4^{th} amylase (Trire2_57128) gene encoding a polypeptide having an amino acid sequence having at least 60%, at least 65%, at least 70%, at least 80%, at least 85%, at least 90%, at least 95%, at least 99% or 100% sequence identity to SEQ ID NO:72 or the mature polypeptide thereof, wherein said amylase genes are modified rendering the mutant strain deficient in their production relative to the parental strain when cultivated under the same conditions.

### EXAMPLES

Aspects of the present teachings may be further understood in light of the Examples, which should not be construed as limiting the present teachings in any way.

### Example 1: Vector for Over-Expression of Bip1 in T. reesei

A Gateway-compatible expression vector, pTrex2g/hygB, was designed to enable over-expression of the T. reesei chaperone gene bip1. After insertion into pTrex2g/hygB the open reading frame of the bip1 gene was flanked by the promoter sequences of the T. reesei pki1 gene and the terminator sequences of the T. reesei cbh1 gene. The vector also contained the E. coli hygromycin phosphotransferase (hph) gene flanked by the promoter sequences of the Neurospora crassa cpc-1 gene and the terminator sequences of the Aspergillus nidulans trpC gene.

The following segments of DNA were assembled in the construction of Trex2g/HygB:

A 728 bp fragment of T. reesei genomic DNA representing the promoter region from the pki1 (pyruvate kinase) gene. At the 5′ end of this DNA were 6 bp of synthetic DNA representing a SpeI restriction site and at the 3′ end were 6 bp of synthetic DNA adding a SacII restriction site.

The 1714 bp Gateway cassette may be used to allow insertion of the chaperone or foldase sequence using Invitrogen Gateway cloning technology (Thermo Fisher Scientific). This cassette has the following components: the 125 bp E. coli attR1 phage λ attachment site, a chloramphenicol resistance gene, the E. coli cedB gene and the 125 bp E. coli attR2 phage λ attachment site.

The Gateway cassette was followed by a 17 bp fragment of synthetic DNA ending with an AscI site. The native T. reesei cbh1 terminator region (356 bp) immediately followed the AscI site. This terminator region ended with 4 bp of synthetic DNA being the half of a Pmel restriction site (GTTT) remaining after digestion.

A 2.6 kb cassette consisting of the Neurospora crassa cpc-1 promoter fused to the E. coli hph open reading frame was followed by the Aspergillus nidulans trpC terminator. This cassette was amplified by PCR from the vector pFAC1 described by Barreau et al. (1998). The PCR product had 55 bp of synthetic DNA (part of a multiple cloning site) at one end and was blunt-end ligated to the digested Pmel site at the end of the cbh1 terminator. At the other end the PCR product had 20 bp of synthetic DNA terminating in a SphI site that was digested to link with pSL1180 below.

The above DNA fragments were inserted in the E. coli vector pSL1180 between the SpeI and SphI sites of the multiple cloning sites.

### Example 2: The Trichoderma reesei Chymosin Production Strain CHY1-2

A synthetic version of the bovine prochymosin B open reading frame (SEQ ID NO: 42) was constructed with codon usage optimized for expression in Trichoderma. A vector, pTrex4-ChyGA was designed for the expression of an open reading frame encoding a fusion protein that consists of the following components from the amino-terminus: the T. reesei CBHI secretion signal sequence, the T. reesei CBHI catalytic core and linker region, and the bovine prochymosin B protein. This open reading frame is flanked by the promoter and terminator sequences of the T. reesei cbh1 gene. The vector also contains the Aspergillus nidulans amdS gene, encoding acetamidase, as a selectable marker for transformation of T. reesei.

The following segments of DNA were assembled in the construction of pTrex4-ChyGA: The T. reesei cbh1 promoter and coding region. This DNA sequence begins at a naturally occurring HindIII site approximately 2250 bp upstream of the coding region. It ends at a SpeI site created at the end of the sequence encoding the CBHI linker region by changing the codon for the threonine residue at position 479 of preCBHI from ACC to ACT and adding AGT nucleotides immediately afterwards.

The synthetic coding region for bovine prochymosin B was directly fused to the end of the CBHI coding region. Immediately after the prochymosin B stop codon are 8 nucleotides of synthetic DNA representing an AscI restriction site (GGCGCGCC).

The native T. reesei cbh1 terminator region (356 bp) immediately followed the above AscI site.

A blunt-end fragment of a 2.75 kb fragment of Aspergillus nidulans genomic DNA including the promoter, coding region and terminator of the amdS (acetamidase) gene was generated by digestions with Ssp1 at naturally occurring restriction sites.

The above DNA fragments were inserted in the E. coli vector pSL1180 (Pharmacia) between the HindIII and StuI sites of the multiple cloning site.

Plasmid pTrex4-CHY GA was inserted into the Trichoderma reesei Morph1 1.1 pyr4+, a strain derived from RL-P37 (Sheir-Neiss, G. and Montenecourt, B. S., 1984, Appl. Microbiol. Biotechnol. 20:46-53) and deleted for the cbh1, cbh2, egl1, and egl2 genes described by Bower et al (Carbohydrases from Trichoderma reesei and other micro-organisms, Royal Society of Chemistry, Cambridge, 1998, p. 327-334) by polyethylene glycol (PEG)-mediated transformation of protoplasts. Transformants were selected on agar medium containing acetamide as sole nitrogen source. This resulted in the chymosin production host strain T. reesei CHY1-2.

### Example 3: Cloning the T. reesei Bip1 Gene and Insertion into pTrex2g/hygB

In order to insert the T. reesei bip1 gene into pTrex2g/HygB the DNA sequence was amplified by PCR using attB PCR primers. The forward primer (F-attB1) had the following sequence at the 5′ end, 5′-GGGGACAAGTTTGTACAAAAAAGCAGGCT-3′, followed by a sequence specific to the 5′ end of the bip1 open reading frame. The reverse primer (R-attB2) had the following sequence at the 5′ end, 5′-GGGGACCACTTTGTACAAGAAAGCTGGGT-3′, followed by a sequence specific to the 3′ end of the bip1 open reading frame. The full sequence of the two primers was:

(SEQ ID NO: 31)
5′-GGGGACAAGTTTGTACAAAAAAGCAGGCTATGGCTCGTTCACGGAG
CTCCC-3′
(SEQ ID NO: 32)
5′-GGGGACCACTTTGTACAAGAAAGCTGGGTTTACAATTCGTCGTGGA
AGTCGCC -3′

The bip1 gene was amplified using Phusion polymerase (Thermo Fisher Scientific) according to the manufacturer's directions. The PCR mixture contained 1 μl T. reesei genomic DNA, 10 μl 5× buffer HF, 1 μl of 10 mM dNTPs, 1.5 μl DMSO, 0.5 μl Phusion DNA polymerase, 2 μl each of the forward and reverse bip1 primers and 32 μl MilliQ H2O. The following temperature and time conditions were used for the PCR. Denaturation of DNA at 98° C. for 30 sec followed by 30 cycles at 98° C. for 10 sec, 55° C. for 30 sec and 72° C. for 90 sec, and a final extension at 72° C. for 10 min.

After agarose gel electrophoresis the 2.3 kb PCR product was purified using a Qiagen QIAquick gel extraction kit (Cat. No. 28706) according to the manufacturer's instructions. The purified PCR product was inserted into the vector pDONR201 (Invitrogen; Cat. No. 11798014) using a BP Clonase reaction (Invitrogen; Cat. No. 11789013) according the following protocol. The following components were mixed; 2 μl pDONR201, 4 μl PCR product, 4 μl BP Enzyme buffer, 6 μl TE buffer, and 4 μl BP Enzyme. After overnight incubation at 25° C. the reaction was stopped by adding Proteinase K solution and incubating for 10 minutes at 37° C. 2 μl of the reaction mixture was used for transformation of E. coli TOP10 chemical competent cells (Invitrogen Cat. No. C4040-10) according to the manufacturer's directions. After sequence analysis, the bip1 sequence was transferred to the expression vector pTrex2g/hygB using the LR Clonase reaction (Invitrogen; Cat. No. 11791019) according to the following protocol. The following components were mixed. 2 μl pDON201R with inserted bip1 gene, 2 μl pTrex2g/hygB, 4 μl LR enzyme buffer, 4 μl LR enzyme mix, and 8 μl TE. Following overnight incubation at 25° C. the reaction was stopped by addition of Proteinase K solution and incubation for 10 minutes at 37° C. 2 μl of the reaction mixture was transformed into E. coli MAX EFFICIENCY DH5a Competent Cells (Invitrogen; Cat. No. 18258012). Plasmid DNA, pTrex2g/HygB/bip1 was isolated from two resulting E. coli colonies for transformation of T. reesei CHY1-2.

### Example 4: Trichoderma Transformation

Expression vector pTrex2g/HygB/bip1 was inserted into spores of T. reesei CHY1-2 using a biolistic transformation procedure. DNA-coated tungsten particles were prepared as follows. 60 mg of M10 tungsten particles were added to 1 ml ethanol (70% or 100%) in a microcentrifuge tube. This mixture was allowed to soak for 15 minutes, followed by centrifugation for 15 min at 15,000 rpm. The supernatant was then decanted and the pellet washed three times with sterile distilled water. The majority of the distilled water was removed after the final wash. The pellet was then resuspended in 1 ml of a 50% glycerol (v/v, sterile) solution. While continuously vortexing a 25 μl aliquot of this particle suspension was removed and placed in a microcentrifuge tube. To this tube the following components were added (while continuously vortexing) in the following order. 0.5-5 μl of pTrex2g/HygB/bip1 DNA solution (1 μg/μl), 25 μl 2.5M CaCl2, and 10 μl 0.1M spermidine.

The mixture was allowed to coat the particles for 5-15 minutes during continuous vortexing and was used as soon as possible to avoid tungsten degradation of the DNA. The mixture was then centrifuged for approximately three seconds. The supernatant was then removed and the pellet was washed with approx 200 μl of 70% ethanol (v/v) followed by a 3 second centrifugation and removal of the supernatant. The pellet was again washed with 200 μl of 100% ethanol, followed by another 3 second centrifugation. The supernatant was removed and the pellet was then resuspended in 24 μl 100% ethanol and mixed by pipetting. 8 μl aliquots were placed onto macrocarrier discs (Bio-Rad, Hercules, CA) by pipetting the aliquots in the exact center of the disks while the disks were in a desiccator. The discs were kept in a desiccator until thoroughly dry and kept there until immediately before use. The macrocarrier discs were inserted into a Model PDS-1000/He Biolistic Particle Delivery System (Bio-Rad, Hercules, CA). This apparatus was used according to the manufacturer's directions to propel the DNA-coated tungsten particles at the T. reesei spores prepared as below.

A spore suspension of strain CHY1-2 was made with approximately 5×10⁸ spores/ml. 100-200 μl aliquots of the spore suspension was spread over an area approximately 6 cm in diameter at the center of a plate of agar medium containing acetamide as sole nitrogen source. After the biolistic transformation, the plates were placed in a 25° C. incubator for 1 day. Then, 1 ml Hygromycin B solution (4 mg/ml) was spread onto the plates and an additional incubation of 3 days at 28° C. was performed. Transformants were transferred onto fresh agar plates with acetamide as sole nitrogen source and Hygromycin B (200 μl/ml), and placed at 28° C.

### Example 5: Chymosin Expression in Shake Flasks

Lactose defined liquid medium (LD) contained the following components. Casamino acids, 9 g/L; (NH4) 2SO4, 5 g/L; MgSO4·7H2O, 1 g/L; KH2PO4, 4.5 g/L; CaCl2.2H2O, 1 g/L, PIPPS, 33 g/L, 400× T. reesei trace elements, 2.5 ml/L; pH adjusted to 5.5 with NaOH. After sterilization, lactose was added to a final concentration of 2% v/v.

400× T. reesei trace elements solution contained the following: citric acid (anhydrous), 175 g/L; FeSO4·7H2O, 200 g/L; ZnSO4·7H2O, 16 g/L; CuSO4·5H2O, 3.2 g/L; MnSO4·H2O, 1.4 g/L; H3BO3, 0.8 g/L.

Ten transformants of T. reesei strain CHY1-2 with the bip1 expression vector were evaluated by shake flask culture in lactose defined liquid medium for improved chymosin production. From each morphologically stable transformant colony on a Petri dish, one square cm was excised and used to inoculate a single 30 ml LD medium in a baffled shake flask. After 3 days of growth at 28° C. and 150 rpm, 5 ml of this pre-culture was used to inoculate 45 ml LD medium in a baffled shake flask. This production culture was grown for 3 days at 28° C. and 150 rpm. Supernatants were collected by centrifugation of the fermentation broth. Chymosin activity was measured and SDS-PAGE and Western analysis were performed to determine the chymosin concentration.

The chymosin activity in the culture supernatant was measured using essentially the same methods as previously described (Dunn-Coleman et al., 1991, Bio/Technology 9:976-981). Two transformants, bip1 #1.2 and bip1 #1.10 were chosen for further study because they showed a significant improvement in chymosin production compared to the host strain T. reesei CHY1-2 (see Table 2, column 2).

Culture supernatants from these two transformants were subjected to SDS-PAGE (sodium dodecyl sulfate-polyacrylamide gel electrophoresis). Following electrophoresis protein was stained with Coomassie Brilliant Blue. Based on the intensity of the 35 kDa band corresponding to mature chymosin transformants bip1 #1.2 and bip1 #1.10 produced more chymosin than strain CHY1-2.

Four replicate shake flask cultures of bip1 #1.2, bip1 #1.10 and strain CHY1-2 were grown and chymosin activity analysis was performed. Again, transformants bip1 #1.2 and bip1 #1.10 clearly produced more active chymosin than the host strain T. reesei CHY1-2 (Table 2, column 3).

TABLE 2
Percentage of chymosin activity in shake flask
supernatants of transformants bip1 #1.2 and bip1
#1.10 compared to T. reesei CHY1-2

| | % of chymosin activity | % of chymosin activity |
|---|---|---|
| Strain | (Single shake flask experiment) | (Average of four flasks) |
| CHY 1-2 | 100 | 100 |
| Bip1 #1.2 | 282 | 363 |
| Bip1 #1.10 | 240 | 370 |

It was possible that some secreted chymosin was present in an inactive form due to degradation. Chymosin was initially secreted as a CBHI-prochymosin fusion protein. At low pH, mature active chymosin was expected to be released by autocatalytic cleavage at the junction between the chymosin pro-region and mature chymosin. Therefore, it was also possible that some chymosin was present as CBHI-prochymosin fusion protein in the culture supernatant and consequently inactive. For these reasons, Western blot analysis was performed to determine the total amount of secreted chymosin; as active, inactive and fused protein. Proteins were separated by SDS-PAGE using the NuPAGE Novex pre-cast gel system according to the manufacturer's instructions (Thermo Fisher Scientific). Following electrophoresis, the proteins were electro-blotted onto a PVDF membrane using an XCell II Blot Module as directed by the manufacturer (Thermo Fisher Scientific). Western Blotting was used to detect alkaline phosphatase-labeled antibodies. Primary antibodies (affinity-purified polyclonal rabbit anti-chymosin) were diluted 1000 times. The blot was scanned and the intensities of the chymosin-specific bands were measured using Total Lab Software. Based on this measure of total chymosin production, transformants bip1 #1.2 and bip1 #1.10 showed a clear increase compared to strain CHY 1-2.

### Example 6: mRNA Analysis of T. reesei CHY1-2 and Bip1 #1.10

Shake flask fermentations were performed to collect mycelium for mRNA level analysis for chymosin, CBHI and Bip1 in two T. reesei strains, CHY1-2 and bip1 #1.10. Broth was collected after 72 hrs of culture and frozen in liquid nitrogen. Total RNA was isolated using a FastRNA Red Kit (Bio 101, Inc., Carlsbad, CA) according to the manufacturer's instructions. In brief, the following protocol was used. Lysing tubes were chilled on dry ice and 500 μl CRSR RED, 500 μl PAR, and 100 μl CIA were added and frozen.

A piece of frozen mycelia (approx. 0.7 cm cubed) was added to the lysing tube with frozen reagents. The tube was placed at 60° C. for 2-5 minutes, until bottom reagents around the beads started to thaw, but not top reagents or sample. The tube was immediately secured in a FastPrep machine and shaken for 3×30 seconds at setting 6, allowing 1 min rest between disruptions. The tubes were removed and placed on wet ice 5 min before centrifugation. The aqueous phase was drawn off to a new tube and an equal volume of CIA was added, vortexed to mix and centrifuged. The last step was repeated and an equal volume of DIPS was added, mixed and incubated at room temperature for 1-2 minutes. The tube was centrifuged to pellet the RNA and the supernatant was removed. The pellet was washed with 500 μl SEWS by adding the wash and removing immediately. The last traces of wash were removed and the pellet was air dried for 5-10 min before resuspending in 200 μl RNase-free water. 40 μl of LiCl solution was added and the sample was incubated at 4° C. overnight. The tube was centrifuged to pellet the RNA, the RNA was washed as before, and finally resuspended in 100-200 μl of RNase-free water.

Complementary DNA (cDNA) synthesis was performed with a High Archive cDNA synthesis kit from Applied Biosystems Inc. according to the manufacturer's directions, after which the cDNA was amplified with gene specific primers (Table 3).

TABLE 3
Gene-specific primers designed for use in TaqMan Gene Expression Assays

| Name | Sequence |
|---|---|
| CBH1 forward | AGTTACCACGAGCGGTAACAG (SEQ ID NO: 33) |
| CBH1 reverse | AAGAGAACTCGTTGCCAAGC (SEQ ID NO: 34) |
| Bip1 forward | CACCAACACCGTCTACGATG (SEQ ID NO: 35) |
| Bip1 reverse | CGTTCTTCTCAATGACCTTGTAG (SEQ ID NO: 36) |
| Chymosin forward | CAGCAAGCTCGTCGGC (SEQ ID NO: 37) |
| Chymosin reverse | GGTACATCTTGCCGTTGATCTC (SEQ ID NO: 38) |

Quantification of the amplified cDNA was performed using the TaqMan Gene Expression Assay kit from Applied Biosystems, Inc. with an Applied Biosystems 7900 HT thermal cycler according the manufacturer's instructions (Thermo Fisher Scientific). In brief, the TaqMan Universal PCR Master Mix, No AmpErase UNG was mixed with 20× TaqMan Gene Expression Assay Mix (containing unlabelled gene-specific primers and TaqMan MGB probe) and cDNA. The following thermal cycler conditions were then applied. Two minutes at 50° C., 10 min at 95° C., and 40 cycles of 15 sec at 95° C., 1 min at 60° C. The bip1, chymosin and cbh1 levels were determined relative to the native T. reesei genes, gpd1 (encoding glyceraldehyde-3-phosphate dehydrogenase) and act1 (encoding actin). For each gene, a cycle threshold value was determined. This value is equivalent to the number of PCR cycles required for a fluorescence signal to be detectable. The difference between the cycle threshold value (ΔCT) for each of bip1, chymosin or cbh1 and either gpd1 or act1 was calculated. The above mRNA analyses showed that bip1, chymosin and cbh1 levels are all increased as a result of bip1 over-expression in transformant bip1 #1.10 compared to strain CHY 1-2.

### Example 7: T. reesei Strain for Chymosin Production

A vector, pCBHI×CBD-Chy, was designed for the expression of an open reading frame encoding a fusion protein that consists of the following components from the amino-terminus: the T. reesei CBHI secretion signal sequence, the full-length T. reesei CBHI mature protein (including catalytic domain, linker region and cellulose binding domain), and the Bos taurus prochymosin B protein. A single codon was altered within the CBHI catalytic domain in order to inactivate the CBHI enzyme. This open reading frame is flanked by the promoter and terminator sequences of the T. reesei cbh1 gene. The vector also contains the Aspergillus nidulans amdS gene, encoding acetamidase, as a selectable marker for transformation of T. reesei.

The following segments of DNA were assembled in the construction of pCBHI×CBD-Chy: the T. reesei cbh1 promoter and coding region. This DNA sequence begins at a naturally occurring XbaI site approximately 1500 bp upstream of the coding region. The following changes to the native T. reesei genomic DNA sequence were made. Within the CBHI coding region the codon for amino acid 212 of the mature CBHI protein was changed from GAG (Glutamic acid) to CAG (Glutamine), known to result in production of an inactive form of CBHI (Stahlberg, J. (1996) J. Mol. Biol. 264:337-349).

Within the segment of the coding region encoding the CBHI linker region a change was made to create a SpeI restriction site. This changed the sequence from ACC CAG to ΔCT AGT ACC CAG (SEQ ID NO: 39) altering the amino acid sequence by insertion of two residues from Thr Gln to Thr Ser Thr Gln. The Gln in this sequence represents the first amino acid of the cellulose binding domain of CBHI.

The synthetic coding region for bovine prochymosin B is directly fused in-frame to the end of the CBHI linker coding region. Immediately after the prochymosin B stop codon are 8 nucleotides of synthetic DNA representing an AscI restriction site (GGCGCGCC).

The native T. reesei cbh1 terminator region (356 bp) immediately follows the above AscI site. This terminator region ends with 4 bp of synthetic DNA being the half of a Pmel restriction site (GTTT) remaining after digestion.

A 2.75 kb fragment of Aspergillus nidulans genomic DNA including the promoter, coding region and terminator of the amdS (acetamidase) gene. This is a blunt-ended fragment generated by digestion with SspI at naturally occurring restriction sites. A natural XbaI site occurs before the SspI site at the end of the terminator region. A 55 bp fragment of the multiple cloning site of pSL1180 from the StuI to the KpnI site.

The above DNA fragments were inserted in the E. coli vector pNEB193 (New England Biolabs, Inc., USA) between the XbaI and KpnI sites of the multiple cloning site. pNEB193 is identical to pUC19 (Yannisch-Perron et al., 1985) except for the addition of several restriction endonuclease sites to the multiple cloning site.

The expression vector pCBHI×CBD-Chy was digested with XbaI to release a fragment of DNA containing only the cbh1 promoter, CHI-prochymosin B coding sequence, cbh1 terminator and A. nidulans amdS gene. Only this XbaI fragment of DNA, not the entire pCBHI×CBD-Chy expression vector, was inserted into the T. reesei production strain.

In more detail, this XbaI fragment contains the following segments of DNA: The T. reesei cbh1 promoter and coding region. This DNA sequence begins at a naturally occurring XbaI site approximately 1500 bp upstream of the coding region. The following changes to the native T. reesei genomic DNA sequence were made.

Within the CBHI coding region the codon for amino acid 212 of the mature CBHI protein was changed from GAG (Glutamic acid) to CAG (Glutamine) resulting in production of an inactive form of CBHI. Within the segment of the coding region encoding the CBHI linker region a change was made to create a SpeI restriction site. This changed the sequence from ACC CAG to ACT AGT ACC CAG altering the amino acid sequence by insertion of two residues from Thr Gln to Thr Ser Thr Gln. The Gln in this sequence represents the first amino acid of the cellulose binding domain of CBHI. At the end of the CBHI coding sequence two additional codons (ACT AGT encoding Thr Ser) were added to create a SpeI restriction site.

The synthetic coding region for bovine prochymosin B was directly fused to the end of the CBHI coding region. Immediately after the prochymosin B stop codon are 8 nucleotides of synthetic DNA representing an AscI restriction site (GGCGCGCC).

The native T. reesei cbh1 terminator region (356 bp) immediately follows the above AscI site. This terminator region ends with 4 bp of synthetic DNA being the half of a Pmel restriction site (GTTT) remaining after digestion. A 2.75 kb fragment of Aspergillus nidulans genomic DNA including the promoter, coding region and terminator of the amdS (acetamidase) gene. This fragment begins at a naturally occurring SspI site and ends at a natural XbaI site.

The expression vector pTrex2g/HygB/Bip1 was described in Example 1.

Plasmid pTrex2g/HygB/Bip1 was digested with SpeI and BmrI and the Bip1 expression cassette was purified by agarose gel electrophoresis. T. reesei strain Pent A (derived from strain RL-P37 with deletions or disruptions in the cbh1, cbh2, egl1, egl2 and egl3 genes) was transformed with a mixture of the purified CBHI-prochymosin B and Bip1 expression cassettes using a PEG-mediated protoplast transformation protocol.

Several transformants were isolated, grown in shake flasks and examined for chymosin production. One transformant was chosen and called strain Trichoderma reesei Pent CHY-Bip 3. The integration of DNA in transformant Pent CHY-Bip 3 was investigated by Southern analysis to show that only the intended modifications to the T. reesei Pent A strain had been made. Chromosomal DNA was extracted from the transformant, as well as from the host strain PentΔ. The chromosomal DNA was digested, independently, with XbaI, SpeI or StuI. The digests were purified and concentrated by ethanol precipitation. Digested DNA (5-10 ug) was subjected to electrophoresis on 1% agarose gels. DNA molecular weight markers, and expression vectors pCBHI×CBD-Chy (digested with XbaI) and pTrex2g/HygB/Bip1 (digested with BmrI), were also run on appropriate gels. Following electrophoresis, DNA was transferred to nylon membrane (Nytran SuperCharge; Schleicher & Schuell BioScience). After blotting, the membranes were hybridized with 32P-labeled pCBHI×CBD-Chy, pTrex2g/HygB/Bip1, pUC18, or a PCR product consisting of the entire Hygromycin B resistance cassette (including cpc-1 promoter, hph coding region, and trpC terminator). The latter PCR product was generated from pTrex2g/HygB/Bip1 as template using the following two primers:

hph1,
(SEQ ID NO: 40)
5′ TCTCCGGTGTCCCTTGTCCCTTC-3′
and
hph2,
(SEQ ID NO: 41)
5′-ACCTGTGGCGCCGGTGATGCCGG-3′.

No hybridizing bands were observed with chromosomal DNA extracted from T. reesei Pent A or transformant Pent CHY-Bip 3 using the pUC18 probe demonstrating that no bacterial vector DNA was integrated in either of these strains. Similarly, hybridization with the Hygromycin B resistance cassette demonstrated that this DNA had not integrated in strain Pent CHY-Bip 3. The hybridization results with pCBHI×CBD-Chy and pTrex2g/HygB/Bip1 demonstrated that both the CBHI-prochymosin B expression cassette and the Bip1 expression cassette were integrated in strain Pent CHY-Bip 3. These results showed that only the intended CBHI-prochymosin B and Bip1 expression cassettes were integrated into the T. reesei chromosome.

### Example 8: Optimization of Chaperone

T. reesei transcript data from RNA-Seq experiments conducted under cellulase-inducing conditions were compared at various times during the fermentation. Total RNA was isolated from a fermentation time-course with a cellulase expressing strain, N3, using TriZOL (Thermo Fisher Scientific). A TruSeq RNA library kit (Illumina, USA) was used to construct sequencing libraries for fermentation time points in two biological replicates. The libraries were sequenced on an Illumina HiSeq 2000 platform. Using the GeneData Analysis vXY platform (GeneData, Switzerland), reads were mapped to the T. reesei JGI v2 genome assembly (JGI Trichoderma reesei genome database v. 2.0 [see genome.jgi.doe.gov]; Martinez et al., 2008, Nature Biotechnol. 26:553-560) using Bowtie2 (Langmead B, Salzberg S. Fast gapped-read alignment with Bowtie 2. Nature Methods. 2012, 9:357-359.). Read counts were quantified using featureCounts (www.rdocumentation.org/packages/Rsubread/versions/1.22.2/topics/featureCounts; Yang Liao, Gordon K Smyth and Wei Shi. featureCounts: an efficient general-purpose program for assigning sequence reads to genomic features. Bioinformatics, 30 (7): 923-30, 2014). The mRNA levels for native bip1 were determined to be substantially higher compared to pki (pyruvate kinase) transcript. See Table 4 below. Transcript data for the respective genes are represented by TPM values (Transcripts per million) for respective 14 L fermentation time points.

TABLE 4
mRNA levels

| | sil1 | bip1 | PPI | pki |
|---|---|---|---|---|
| t = 31 hr | 26 | 475 | 978 | 113 |
| t = 113 hr | 70 | 1427 | 9672 | 23 |
| t = 160 hr | 59 | 1726 | 5210 | 35 |

A T. reesei (Δcbh1, Δcbh2, Δegl1, Δegl2) strain derived from RL-P37 was transformed with a CBHI core-prochymosin B fusion cassette (essentially as described in Example 2 above) and one of several native ER chaperone expression cassettes including, Sill, Bip1, and PPI (prolyl peptidyl isomerase) using PEG-mediated protoplast transformation. The bip1 expression cassettes contained either the native bip1 promoter or the promoter of pyruvate kinase (pki). The pyruvate kinase promoter has historically been used in Trichoderma expression as a weak constitutive promoter. Co-expression with the bip1 with the native promoter resulted in the highest level of chymosin production, as determined by SDS-PAGE from MTP-grown cultures (WO2014047520, incorporated here by reference). See Table 5 below.

TABLE 5
Over-expression of Chymosin and Chaperones

| CBHI-core_chymosin | Relative chymosin |
|---|---|
| expressed with: | expression level |
| None (native copy only) | + |
| Bip1 with native bip1 promoter | ++++ |
| PPI (prolyl peptidyl isomerase) | ++ |
| Sil1 (nucleotide exchange factor) | +++ |
| Bip1 with pki promoter | ++ |

### Example 10: Low Side Activity T. reesei Host

The deletion or disruption of cellulase genes in T. reesei (Δcbh1, Δcbh2, Δegl1, Δegl2) were described in U.S. Pat. No. 5,650,322, incorporated herein by reference. The deletion of cbh1 additionally resulted in deletion of the protease gap1 (Trire2_69555) (Nordberg H, Cantor M, Dusheyko S, Hua S, Poliakov A, Shabalov I, Smirnova T, Grigoriev I V, Dubchak I. Nucleic Acids Res. 2014, 42 (1): D26-31). A T. reesei (Δcbh1, Δcbh2, Δegl1, Δegl2) strain derived from RL-P37 was transformed with a CBHI core-prochymosin B fusion cassette (essentially as described in Example 2 above) to generate the 4d-cell strain in Table 7. Secreted enzymatic side activities were further reduced by sequential gene deletion of additional endoglucanases, mannanase, xylanases, and beta-glucosidase: egl5 (WO2012/054554 A2), egl3, egl4, egl6, man1 (WO2015/020876 A1, incorporated by reference herein), xyn2 (WO2015/114108 A1), xyn3 and bgl1 (WO2016/100272 A1), yielding the host strain Cellulighter.

### Example 11: Impact of Low Side Activity on Hydrocolloid Viscosity

DNA encoding bovine prochymosin B was synthesized by GeneArt (USA) and expressed as a C-terminal fusion to the catalytically inactive CBHI core protein, under control of the native T. reesei cbh1 promoter and terminator, using the T. reesei pyr2 gene (orotate phosphoribosyl transferase) as a selectable marker. On the same expression cassette, bip1 chaperone was under control of the native bip1 promoter. The expression cassette was amplified by PCR. A second copy of bip1, under control of the native bip1 promoter was also amplified and both cassettes were co-transformed into the Cellulighter strain by PEG-mediated protoplast transformation. Transformants were selected on agar plates with minimal media without uridine. Transformants were inoculated into liquid culture (WO2014047520, incorporated herein by reference) and fermented in a shaking incubator for 4 days. Cellulighter CHY-Bip culture supernatants were analyzed by SDS-PAGE for expression. Milk clotting activity was determined as previously described and a high activity clone was chosen for further characterization. Genome sequencing confirmed a lower protease background in this clone due to deletion of sedolisin genes tpp1 (Trire2_82623) and sed2 (Trire2_70962).

Enzymatic side activities were detected and quantified by determining the remaining viscosity, after enzyme exposure, of hydrocolloid cellulase, mannanase, and pectinase substrates in the manner described in WO2011107472, using Microlab® STAR™ liquid handler with Total Aspiration Dispense Monitoring (TADM) software (Hamilton Company) and the following modifications (see Table 6 below):

TABLE 6
Modifications

| Liquid-handler model | Hamilton Microlab STAR | |
|---|---|---|
| Tip type | 300-μL standard volume, | |
| | unmodified, capacitive | |
| Volume aspirated | 150 | μL |
| Liquid Level Sensing | Capacitive; 2 mm below liquid | |
| | surface (following during | |
| | aspiration and dispense) | |
| Flow rate (aspiration and dispense) | 20 | μL/second |
| Mix flow rate (aspiration and dispense) | 100 | μL/second |
| Blowout volume (aspiration and | 0 | μL |
| dispense) | | |
| Swap speed (aspiration and dispense) | 15 | mm/second |
| Settling time (aspiration) | 1 | second |

Relative viscosity was measured as the negative pressure value at the 7000-millisecond timepoint (the median value between 6800 and 7200 milliseconds) in the TADM aspiration pressure curve.

Cellulase, mannanase, and pectinase-degrading activities were reported as “percent viscosity remaining relative to blank,” with 100% viscosity remaining represented by a well containing hydrocolloid substrate with a water blank added, and 0% viscosity remaining represented by a well containing water without substrate.

### Substrate Preparation

Hydrocolloid substrates were buffered using McIlvaine buffer, comprising two stock solutions, A and B, which were combined as described for each substrate.
Stock solution A: 0.1M citric acid (21.0 g C6H807*H2O/1 L MilliQ water)
Stock solution B: 0.2M disodium hydrogen phosphate (35.6 g Na2HPO4*2H2O/1 L MilliQ water)

To prepare 0.5% carboxymethyl cellulose (CMC) substrate (per 100 mL): Sterile water (80 ml) was combined with 0.5 g of CMC 1250 (e.g. CMC CEKOL 10000 from Nouryon Functional Chemicals or Blanose 9H4F from Ashland Industries Europe GMBH) or Grinsted® Cellulose gum MAS 250 (aka Texturecel 40000 PA Sodium, IFF Nutrition and Biosciences) and stirred. The mixture was heated to dissolve the CMC, cooled, and then 5 mL stock solution A and 15 mL stock solution B were added with stirring.

To prepare 0.5% guar gum substrate (per 100 mL): Sterile water (80 ml) was combined with 0.5 g of Grinsted® Guar 200 (Danisco Switerzerland AG). The slurry was heated while stirring, forming a homogeneous suspension. Once the suspension cooled, 5 mL stock solution A and 15 mL stock solution B were added with stirring.

To prepare 1.4% pectin substrate (per 100 mL): Sterile water (80 ml) was heated and 1.4 g of Pectin AMD783 (IFF Nutrition and Biosciences, Grindsted, Denmark) were added gradually with stirring. Once the suspension cooled, 12.29 mL stock solution A and 7.71 mL stock solution B were added with stirring.

### Reactions

1 mL of hydrocolloid substrate was dispensed to each well of a deep-well 96-well plate with round-bottom, square polypropylene wells (Corning Life Sciences, Tewksbury, MA) using a Matrix Wellmate multichannel peristaltic dispenser (Thermo Scientific, Waltham, MA). 20 μL of enzyme sample or water blank were added per well using a Biomek liquid-handling robot (Beckman Coulter, Brea, CA). Enzyme sample dose was based on chymosin activity. Culture broths from strains with different side activity deletions were compared. Commercially available Chy-Max® Extra chromatographically purified chymosin (Christian Hansen) was used as a reference. The deep-well plates were thermally sealed and shaken for 20 hours at 37° C., 300 RPM, 0.25 cm throw. The plates were mixed on orbital benchtop shakers for an additional hour while they cooled to ambient room temperature, then placed on the Hamilton Microlab STAR for viscometric determination.

### Results

Set forth below in Table 7 are the results showing the impact of strain background on the selected cellulase substrates. Use of the Δcbh1, Δcbh2, Δegl1, Δegl2, Δgap1, Δegl5, Δegl3, Δegl4, Δegl6, Δman1, Δxyn2, Δxyn3, Δbgl1, Δtpp1, Δsed2 T. reesei shows a marked improvement in cellulase activity (i.e., less cellulase activity and greater remaining hydrocolloid viscosity) as compared to its predecessors. Genetic deletion achieved low undesired cellulase side activity without costly purification; For example, compare Table 7 experimental row 2 to row 5.

TABLE 7
Cellulase Substrate Remaining Viscosity

| | | | Dose | % remaining |
|---|---|---|---|---|
| | | | (chymosin | viscosity |
| Chymosin Strain | Deletion genotype | Cellulase substrate | activity U/ml) | (n = 2) |
| Commercial | Chy-Max Extra ® | Cellulose gum MAS | 65 | 94 +/− 4 |
| Aspergillus niger | | 550 | | |
| chromatographically | | | | |
| purified chymosin | | | | |
| product (Christian | | | | |
| Hansen). See | | | | |
| Berka, R M, et al. | | | | |
| (1991) Food | | | | |
| Biotechnology Vol. | | | | |
| 19, pp. 681-685. | | | | |
| Commercial | Chy-Max Extra ® | Cellulose gum MAS | 100 | 90 +/− 13 |
| Aspergillus niger | | 550 | | |
| chromatographically | | | | |
| purified chymosin | | | | |
| product (Christian | | | | |
| Hansen). See Berka, | | | | |
| R M, et al. (1991) | | | | |
| Food Biotechnology | | | | |
| Vol. 19, pp. 681- | | | | |
| 685. | | | | |
| T. reesei 4d-cell | Δcbh1, Δchh2, Δegl1, | Cellulose gum MAS | 80 | 1 +/− 0 |
| unpurified | Δegl2, Δgap1 | 550 | | |
| chymosin broth | | | | |
| T. reesei Pent CHY- | Δchh1, Δcbh2, Δegl1, | Cellulose gum MAS | 79 | 2 +/− 0 |
| Bip 3 unpurified | Δegl2, Δgap1, Δegl3 | 550 | | |
| broth | | | | |
| T. reesei | Δcbh1, Δcbh2, Δegl1, | Cellulose gum MAS | 100 | 79 +/− 1 |
| Cellulighter CHY- | Δegl2, Δgap1, Δegl5, | 550 | | |
| Bip unpurified broth | Δegl3, Δegl4, Δegl6, | | | |
| | Δman1, Δxyn2, Δxyn3, | | | |
| | Δbgl1, Δtpp1, Δsed2 | | | |
| T. reesei Pent CHY- | Δcbh1, Δchh2, Δegl1, | CMC 1250 | 79 | 2 +/− 0 |
| Bip 3 unpurified | Δegl2, Δgap1, Δegl3 | | | |
| broth | | | | |
| T. reesei | Δcbh1, Δcbh2, Δegl1, | CMC 1250 | 100 | 75 +/− 3 |
| Cellulighter CHY- | Δegl2, Δgap1, Δegl5, | | | |
| Bip unpurified broth | Δegl3, Δegl4, Δegl6, | | | |
| | Δman1, Δxyn2, Δxyn3, | | | |
| | Δbgl1, Δtpp1, Δsed2 | | | |

Set forth below in Table 8 are the results showing the impact of strain background on the selected mannanase substrate. Use of the Δcbh1, Δcbh2, Δegl1, Δegl2, Δgap1, Δegl5, Δegl3, Δegl4, Δegl6, Δman1, Δxyn2, Δxyn3, Δbgl1, Δtpp1, Δsed2 T. reesei shows a marked improvement in mannanase activity as compared to its predecessors.

TABLE 8
Mannanase Substrate Remaining Viscosity

| | | | Dose | % remaining |
|---|---|---|---|---|
| | | Mannanase | (chymosin | viscosity |
| | Chymosin strain deletion genotype | substrate | activity U/ml) | (n = 2) |
| T. reesei 4d-cell | Δcbh1, Δcbh2, Δegl1, Δegl2, | Guar 200 | 80 | 2 +/− 1 |
| chymosin broth | Δgap1 | | | |
| T. reesei | Δcbh1, Δchh2, Δegl1, Δegl2, | Guar 200 | 100 | 100 +/− 2 |
| Cellulighter | Δgap1, Δegl5, Δegl3, Δegl4, Δegl6, | | | |
| CHY-Bip broth | Δman1, Δxyn2, Δxyn3, Δbgl1, | | | |
| | Δtpp1, Δsed2 | | | |

### Example 12: Bip1 and Chymosin Homolog Expression

Chymosin expression vectors were constructed by using the GeneArt™ Seamless Cloning and Assembly Kit (Thermofisher, Waltham U.S.), assembling the following two PCR products: (1) the vector backbone contains Trichoderma reesei cbh1 core (E212Q) and linker flanked by the native cbh1 promoter and terminator, Trichoderma reesei pyr2 marker downstream of terminator, and native expression cassette Trichoderma reesei bip1 chaperone upstream of promoter; (2) chymosin prosequence (lacking signal peptide) amplified from synthetic DNA synthesized by Twist Bioscience (San Francisco, U.S.) that contains homology to Trichoderma reesei cbh1 linker at the 5′ end and Trichoderma reesei cbh1 terminator at the 3′ end for assembly of the CBHI-chymosin fusion. PCR reactions were carried out using Q5 High-Fidelity DNA Polymerase (NEB-Ipswich, U.S.) according to standard protocol. The assembled product was added to 50 μL TOP10 Chemically Competent E. coli (Thermo Fisher Scientific) and transformation was carried out according to standard protocol. Plasmid DNA was isolated from E. coli colonies using the NucleoSpin Plasmid Mini Kit (Macherey-Nagel, Düren, Germany) according to standard protocol.

CRISPR-Cas9 technology was used to generate a double strand break (DSB) at a specific genomic position that stimulated DNA repair and integration of the expression cassette. The DSB was generated via in vitro assembled RNP (ribonucleoprotein) complexes that specifically cut the T. reesei genome to introduce the donor DNA fragments of the Chymosin expression cassettes. PEG-mediated transformation was used to introduce the expression vector into protoplasts of a Trichoderma reesei strain Cellulighter. The donor DNA amplified from the chymosin expression vectors contained the expression cassette and the pyr2 marker. The expression cassette comprised of chymosin that was fused to the Trichoderma reesei cbh1 core (inactive) and linker, flanked by the Trichoderma reesei cbh1 promoter and cbh1 terminator, pyr2 marker and bip1 chaperone. Vectors were also constructed lacking the bip1 chaperone cassette.

To generate the Cas9-RNP, EnGen® Spy Cas9 NLS (NEB-Ipswich, U.S.), and site-specific guide RNA (gRNA) from Synthego (Redwood City, U.S.) were combined using the reaction conditions recommended. For the transformation reaction 2 μl of Cas9-RNP were combined with approximately 1 ug of donor DNA. Transformants were selected on minimal media agarose overlays without uridine in 24-well plates. Once transformants were visible, they were transferred into a 24-well plate containing minimal media agar without uridine and incubated in a light cycling incubator for 5 days. Once sporulation occurred, spores were harvested and inoculated in triplicate into liquid culture (WO2014047520, incorporated herein by reference) and fermented in a shaking incubator for 4 days. Cultures were then filtered through a 0.2 μm filter and supernatants analyzed by SDS-PAGE for expression and by reverse phase HPLC for specific determination of the chymosin concentration.

Set forth below in Table 9 is the expression levels of various chymosins from different species plus and minus Bip1. Bip1 enhances chymosin levels from all species.

TABLE 9
Chymosin Homolog Expression

| Chymosin Sample | mg/L chymosin |
|---|---|
| B. taurus chymosin | 142 +/− 14 |
| B. taurus chymosin (Bip1) | 444 +/− 19 |
| C. dromedarius chymosin | 161 +/− 19 |
| C. dromedarius chymosin (Bip1) | 691 +/− 38 |
| L. glama chymosin | 88 +/− 1 |
| L. glama chymosin (Bip1) | 195 +/− 6 |
| V. pacos chymosin | 202 +/− 9 |
| V. pacos chymosin (Bip1) | 710 +/− 3 |

### Example 13: Lower Side Activity T. reesei Host

RNAseq and protein mass spectrometry secretome analyses were used to identify polygalacturonase genes for reduction of pectin degrading side activity. Two genes encoding putative polygalacturonases, Trire2_103049 (pec2) and Trire2_112140 (pec1), were individually deleted from a T. reesei chymosin-expressing parent strain (Δcbh1, Δcbh2, Δegl2, Δegl1, Δgap1, Δpyr2, Δegl5, Δman1, Δegl6, Δegl3, Δegl4). Table 10 below shows the loss of pectinase in the pec1 and pec2 deleted strains.

TABLE 10
Pectinase acitivty of strain extracts

| Sample | % Remaining viscosity |
|---|---|
| Parent strain | 46 +/− 0.8% |
| Δpec1 | 50 +/− 0.4% |
| Δpec2 | 86 +/− 0.2% |
| No enzyme blank | 100 +/− 0.5% |

### Example 14: Reduced Starch Degrading Side Activity

Genomic analysis was used to predict amylase genes for deletion to reduce starch degrading side activity. Alpha-amylase Trire2_105956 was deleted a T. reesei Cellulighter chymosin-expressing strain. Culture broth from the deleted strain was compared to the broth from the parent strain, cultured in the same manner. Broths were spotted onto a starch agar plate based on equal chymosin activity. The deleted strain broth produced a smaller clearing zone when the plate was flooded with iodine solution to aid visualization. Amylase activity was reduced but not completely removed from the deletion strain, so other amylases could additionally be deleted. Some candidate genes for deletion include Trire2_123368, Trire2_1885, and Trire2_57128.

What is claimed is:

1. An engineered Trichoderma filamentous fungus host cell comprising an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene.

2. The host cell of claim 1 wherein the secretion enhancing protein is bip1, ppi1 or sil1.

3. The host cell of claim 2 wherein said bip1 protein comprises a polypeptide having an amino acid sequence at least 60%, at least 65%, at least 70%, at least 80%, at least 85%, at least 90%, at least 95%, at least 99% or 100% sequence identity to SEQ ID NO:16 or a mature version thereof, said ppi1 comprises a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 comprises a polypeptide having an amino acid sequence at least 60%, at least 65%, at least 70%, at least 80%, at least 85%, at least 90%, at least 95%, at least 99% or 100% sequence identity to SEQ ID NO: 30 or a mature version thereof.

4. The host cell of claim 3 wherein the secretion enhancing protein is bip1 comprising a polypeptide having an amino acid sequence according to SEQ ID NO:16 or a mature version thereof.

5. The host cell of any of claims 1 to 4 wherein the Trichoderma host cell is selected from the group consisting of Trichoderma harzianum, Trichoderma koningii, Trichoderma longibrachianun, Trichoderma reesei, and Trichoderma viride.

6. The host cell of claim 5 wherein the Trichoderma host cell is Trichoderma reesei.

7. The host cell of any of claims 1 to 6 further comprising a heterologous gene expressing a secretable polypeptide.

8. The host cell of claim 7 wherein the secretable polypeptide is chymosin.

9. The host cell of claim 8 wherein the chymosin is bovine chymosin, camel chymosin, llama chymosin or alpaca chymosin.

10. The host cell of claim 9 wherein said bovine chymosin comprises a polypeptide having an amino acid sequence at least 60%, at least 65%, at least 70%, at least 80%, at least 85%, at least 90%, at least 95%, at least 99% or 100% sequence identity to SEQ ID NO:48, said camel chymosin comprises a polypeptide having an amino acid sequence at least 60%, at least 65%, at least 70%, at least 80%, at least 85%, at least 90%, at least 95%, at least 99% or 100% sequence identity to SEQ ID NO:49, said llama chymosin comprises a polypeptide having an amino acid sequence at least 60%, at least 65%, at least 70%, at least 80%, at least 85%, at least 90%, at least 95%, at least 99% or 100% sequence identity to SEQ ID NO:50 and said alpaca chymosin comprises a polypeptide having an amino acid sequence at least 60%, at least 65%, at least 70%, at least 80%, at least 85%, at least 90%, at least 95%, at least 99% or 100% sequence identity to SEQ ID NO:51.

11. The host cell of claim 9 wherein the chymosin is bovine chymosin.

12. The host cell of claim 11 wherein said bovine chymosin comprises a polypeptide having an amino acid sequence according to SEQ ID NO:48.

13. The host cell of any of claims 8 to 12 wherein the chymosin is expressed through a Trichoderma reesei promoter.

14. The host cell of claim 13 wherein the chymosin is expressed under a cbh1 promoter.

15. The host cell of claim 14 wherein the chymosin is produced as a fusion protein.

16. The host cell of claim 15 wherein the chymosin is produced as a fusion protein with a CBHI, or a portion thereof.

17. The host cell of claim 16 wherein the chymosin is produced as a fusion protein with a CBHI, or a portion thereof, and the CBHI amino acid sequence is altered to reduce or eliminate catalytic activity.

18. Use of a host cell according to any of claims 8 to 17 to produce chymosin.

19. A method for production of a secretable polypeptide in an engineered Trichoderma filamentous fungus host cell comprising an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene, the method comprising:
expressing a heterologous gene in said host cell to provide said secretable polypeptide.

20. The method of claim 19 wherein the secretion enhancing protein is bip1, ppi1 or sil1.

21. The method of claim 20 wherein said bip1 protein comprises a polypeptide having an amino acid sequence at least 60%, at least 65%, at least 70%, at least 80%, at least 85%, at least 90%, at least 95%, at least 99% or 100% sequence identity to SEQ ID NO: 16 or a mature version thereof, said ppi1 comprises a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 comprises a polypeptide having an amino acid sequence at least 60%, at least 65%, at least 70%, at least 80%, at least 85%, at least 90%, at least 95%, at least 99% or 100% sequence identity to SEQ ID NO: 30 or a mature version thereof.

22. The method of claim 20 wherein the secretion enhancing protein is bip1.

23. The method of claim 22 wherein said bip1 protein comprises a polypeptide having an amino acid sequence according to SEQ ID NO:16 or a mature version thereof.

24. The method of any of claims 19 to 23 wherein the Trichoderma host cell is selected from the group consisting of Trichoderma harzianum, Trichoderma koningii, Trichoderma longibrachianim, Trichoderma reesei, and Trichoderma viride.

25. The method of claim 24 wherein the Trichoderma host cell is Trichoderma reesei.

26. The method of any of claims 19 to 25 wherein the secretable polypeptide is chymosin.

27. The method of claim 26 wherein the chymosin is bovine chymosin, camel chymosin, llama chymosin or alpaca chymosin.

28. The method of claim 27 wherein said bovine chymosin comprises a polypeptide having an amino acid sequence at least 60%, at least 65%, at least 70%, at least 80%, at least 85%, at least 90%, at least 95%, at least 99% or 100% sequence identity to SEQ ID NO:48, said camel chymosin comprises a polypeptide having an amino acid sequence at least 60%, at least 65%, at least 70%, at least 80%, at least 85%, at least 90%, at least 95%, at least 99% or 100% sequence identity to SEQ ID NO:49, said llama chymosin comprises a polypeptide having an amino acid sequence at least 60%, at least 65%, at least 70%, at least 80%, at least 85%, at least 90%, at least 95%, at least 99% or 100% sequence identity to SEQ ID NO:50 and said alpaca chymosin comprises a polypeptide having an amino acid sequence at least 60%, at least 65%, at least 70%, at least 80%, at least 85%, at least 90%, at least 95%, at least 99% or 100% sequence identity to SEQ ID NO:51.

29. The method of claim 26 wherein the chymosin is bovine chymosin.

30. The method of claim 26 wherein said bovine chymosin comprises a polypeptide having an amino acid sequence according to SEQ ID NO:48.

31. The method of any of claims 26 to 30 wherein the chymosin is expressed through a Trichoderma reesei promoter.

32. The method of claim 31 wherein the chymosin is expressed under a cbh1 promoter.

33. The method of any of claims 26 to 32 wherein the chymosin is produced as a fusion protein.

34. The method of claim 33 wherein the chymosin is produced as a fusion protein with a CBHI, or a portion thereof.

35. The method of claim 34 wherein the chymosin is produced as a fusion protein with a CBHI, or a portion thereof, and the CBHI amino acid sequence is altered to reduce or eliminate catalytic activity.

36. The host cell of any of claims 8 to 17, wherein the secretion level of the chymosin in the cell is at least 50 mg/liter when the host cell grows in a fermentation condition.

37. A supernatant obtained from a culture of the host cell of claim 36, wherein the supernatant contains substantial amount of chymosin, but not a substantial amount of the host cell.

38. A supernatant obtained using the method of any of claims 26 to 35, wherein the supernatant contains substantial amount of chymosin, but not a substantial amount of the filamentous fungus.

39. An isolated mutant of a parental Trichoderma strain expressing a heterologous polynucleotide and one or more enzyme genes selected from the group consisting of a 1^{st} enzyme (cbh1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:52 or the mature polypeptide thereof, a 2^{nd} enzyme (cbh2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:53 or the mature polypeptide thereof, a 3^{rd} enzyme (egl1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:54 or the mature polypeptide thereof, a 4^{th} enzyme (egl2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:55 or the mature polypeptide thereof, a 5^{th} enzyme (gap1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:65 or the mature polypeptide thereof, a 6^{th} enzyme (egl5) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:58 or the mature polypeptide thereof, a 7^{th} enzyme (egl3) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:56 or the mature polypeptide thereof, a 8^{th} enzyme (egl4) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:57 or the mature polypeptide thereof, a 9^{th} enzyme (egl6) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:59 or the mature polypeptide thereof, a 10^{th} enzyme (man1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:60 or the mature polypeptide thereof, a 11^{th} enzyme (xyn2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:61 or the mature polypeptide thereof, a 12^{th} enzyme (xyn3) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:62 or the mature polypeptide thereof; a 13^{th} enzyme (bgl1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:63 or the mature polypeptide thereof, a 14^{th} enzyme (tpp1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:64 or the mature polypeptide thereof, and a 15^{th} enzyme (sed2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:66 or the mature polypeptide thereof, wherein said one or more enzyme genes are modified rendering the mutant strain deficient in their production relative to the parental strain when cultivated under the same conditions.

40. The isolated mutant of a parental Trichoderma strain according to claim 39 having a 1^{st} enzyme (cbh1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:52 or the mature polypeptide thereof, a 2^{nd} enzyme (cbh2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:53 or the mature polypeptide thereof, a 3^{rd} enzyme (egl1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:54 or the mature polypeptide thereof, a 4^{th} enzyme (egl2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:55 or the mature polypeptide thereof, a 5^{th} enzyme (gap1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:65 or the mature polypeptide thereof, a 6^{th} enzyme (egl5) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:58 or the mature polypeptide thereof, a 7^{th} enzyme (egl3) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:56 or the mature polypeptide thereof, a 8^{th} enzyme (egl4) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:57 or the mature polypeptide thereof, a 9^{th} enzyme (egl6) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:59 or the mature polypeptide thereof, a 10^{th} enzyme (man1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:60 or the mature polypeptide thereof, a 11^{th} enzyme (xyn2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:61 or the mature polypeptide thereof, a 12^{th} enzyme (xyn3) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:62 or the mature polypeptide thereof; a 13^{th} enzyme (bgl1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:63 or the mature polypeptide thereof, a 14^{th} enzyme (tpp1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:64 or the mature polypeptide thereof, and a 15^{th} enzyme (sed2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:66 or the mature polypeptide thereof, wherein said enzyme genes are modified rendering the mutant strain deficient in their production relative to the parental strain when cultivated under the same conditions.

41. The isolated mutant of a parental Trichoderma strain according to claim 39 or 40 further comprising an endogenous secretion enhancing protein gene under the control of a native promoter coding for a secretion enhancing protein, and an exogenously introduced secretion enhancing protein gene expressing said secretion enhancing protein under the control of said native promoter wherein the level of said secretion enhancing protein in said host cell is increased as compared with a corresponding host cell not having the exogenously introduced secretion enhancing protein gene.

42. The isolated mutant of a parental Trichoderma strain of claim 41 wherein the secretion enhancing protein is bip1, ppi1 or sil1.

43. The isolated mutant of a parental Trichoderma strain of claim 42 wherein said bip1 protein comprises a polypeptide having an amino acid sequence at least 60%, at least 65%, at least 70%, at least 80%, at least 85%, at least 90%, at least 95%, at least 99% or 100% sequence identity to SEQ ID NO: 16 or a mature version thereof, said ppi1 comprises a polypeptide having an amino acid sequence at least 60%, at least 65%, at least 70%, at least 80%, at least 85%, at least 90%, at least 95%, at least 99% or 100% sequence identity to SEQ ID NO:25 or a mature version thereof and said sil1 comprises a polypeptide having an amino acid sequence at least 60%, at least 65%, at least 70%, at least 80%, at least 85%, at least 90%, at least 95%, at least 99% or 100% sequence identity to SEQ ID NO:30 or a mature version thereof.

44. The isolated mutant of a parental Trichoderma strain of claim 42 wherein said secretion enhancing protein is bip1.

45. The isolated mutant of a parental Trichoderma strain of claim 44 wherein said bip1 protein comprises a polypeptide having an amino acid sequence according to SEQ ID NO:16 or a mature version thereof.

46. The isolated mutant of a parental Trichoderma strain according to any of claims 39 to 45 wherein the Trichoderma strain is selected from the group consisting of Trichoderma harzianum, Trichoderma koningii, Trichoderma longibrachiatum, Trichoderma reesei, and Trichoderma viride.

47. The isolated mutant of a parental Trichoderma strain according to claim 46 wherein the Trichoderma host cell is Trichoderma reesei.

48. The isolated mutant of a parental Trichoderma strain according to any of claims 39 to 47 further comprising a heterologous polynucleotide encoding chymosin.

49. The isolated mutant of a parental Trichoderma strain according to claim 48 wherein the chymosin is bovine chymosin, camel chymosin, llama chymosin or alpaca chymosin.

50. The isolated mutant of a parental Trichoderma strain according to claim 49 wherein said bovine chymosin comprises a polypeptide having an amino acid sequence at least 60%, at least 65%, at least 70%, at least 80%, at least 85%, at least 90%, at least 95%, at least 99% or 100% sequence identity to SEQ ID NO:48, said camel chymosin comprises a polypeptide having an amino acid sequence at least 60%, at least 65%, at least 70%, at least 80%, at least 85%, at least 90%, at least 95%, at least 99% or 100% sequence identity to SEQ ID NO:49, said llama chymosin comprises a polypeptide having an amino acid sequence at least 60%, at least 65%, at least 70%, at least 80%, at least 85%, at least 90%, at least 95%, at least 99% or 100% sequence identity to SEQ ID NO:50 and said alpaca chymosin comprises a polypeptide having an amino acid sequence at least 60%, at least 65%, at least 70%, at least 80%, at least 85%, at least 90%, at least 95%, at least 99% or 100% sequence identity to SEQ ID NO:51.

51. The isolated mutant of a parental Trichoderma strain according to claim 48 wherein the chymosin is bovine chymosin.

52. The isolated mutant of a parental Trichoderma strain according to claim 51 wherein said bovine chymosin comprises a polypeptide according to SEQ ID NO:48.

53. The isolated mutant of a parental Trichoderma strain according to any of claims 48 to 52 wherein the chymosin is expressed through a Trichoderma promoter.

54. The isolated mutant of a parental Trichoderma strain according to claim 53 wherein the Trichoderma promoter is a Trichoderma reesei promoter.

55. The isolated mutant of a parental Trichoderma strain according to claim 54 wherein the chymosin is expressed under a cbh1 promoter.

56. The isolated mutant of a parental Trichoderma strain according to claim 55 wherein the chymosin is produced as a fusion protein.

57. The isolated mutant of a parental Trichoderma strain according to claim 56 wherein the chymosin is produced as a fusion protein with a CBHI, or a portion thereof.

58. The isolated mutant of a parental Trichoderma strain according to claim 57 wherein the chymosin is produced as a fusion protein with a CBHI, or a portion thereof, and the CBHI amino acid sequence is altered to reduce or eliminate catalytic activity.

59. Use of an isolated mutant of a parental Trichoderma strain according to any of claims 48 to 58 to produce chymosin.

60. The isolated mutant of a parental Trichoderma strain according to any of claims 48 to 58 further comprising one or more pectinase genes selected from the group consisting of a 1^{st} pectinase (pec1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:67 or the mature polypeptide thereof and a 2^{nd} pectinase (pec2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:68 or the mature polypeptide thereof, wherein said one or more pectinase genes are modified rendering the mutant strain deficient in their production relative to the parental strain when cultivated under the same conditions.

61. The isolated mutant of a parental Trichoderma reesei strain according to 60 comprising a 1^{st} pectinase (pec1) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:67 or the mature polypeptide thereof and a 2^{nd} pectinase (pec2) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:68 or the mature polypeptide thereof, wherein said pectinase genes are modified rendering the mutant strain deficient in their production of said 1^{st} and said 2^{nd} pectinase genes relative to the parental strain when cultivated under the same conditions.

62. The isolated mutant of a parental Trichoderma reesei strain according to any of claims 48 to 58 further comprising one or more amylase genes selected from the group consisting of a 1^{st} amylase (Trire2_105956) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:69 or the mature polypeptide thereof and a 2^{nd} amylase (Trire2_123368) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:70 or the mature polypeptide thereof, a 3^{rd} amylase (Trire2_1885) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:71 or the mature polypeptide thereof and a 4^{th} amylase (Trire2_57128) gene encoding a polypeptide comprising an amino acid sequence having at least 60%, at least 65%, at least 70%, at least 80%, at least 85%, at least 90%, at least 95%, at least 99% or 100% sequence identity to SEQ ID NO: 72 or the mature polypeptide thereof, wherein said one or more amylase genes are modified rendering the mutant strain deficient in their production relative to the parental strain when cultivated under the same conditions.

63. The isolated mutant of a parental Trichoderma reesei strain according to 62 comprising a 1^{st} amylase (Trire2_105956) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:69 or the mature polypeptide thereof and a 2^{nd} amylase (Trire2_123368) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:70 or the mature polypeptide thereof, a 3^{rd} amylase (Trire2_1885) gene encoding a polypeptide comprising an amino acid sequence having at least 60%, at least 65%, at least 70%, at least 80%, at least 85%, at least 90%, at least 95%, at least 99% or 100% sequence identity to SEQ ID NO:71 or the mature polypeptide thereof and a 4^{th} amylase (Trire2_57128) gene encoding a polypeptide comprising an amino acid sequence having at least 60%, at least 65%, at least 70%, at least 80%, at least 85%, at least 90%, at least 95%, at least 99% or 100% sequence identity to SEQ ID NO: 72 or the mature polypeptide thereof, wherein said amylase genes are modified rendering the mutant strain deficient in their production relative to the parental strain when cultivated under the same conditions.


SEQUENCE LISTING (72 sequences)

SEQ ID NO: 1 - DNA, 2267 bp, Trichoderma reesei
  source 1..2267: mol_type = other DNA
atggctcgttcacggagctccctggccctcgggctgggcctgctctgctggatcacgctgctcttcgctcctctggcgtttgtcggaaaggccaatgccgcgagcgacgacgcggacaactacggcactgttatcggaattgtaagtcgactgacggcagcaaccccgccattttcttggtgttgatgctcaggcagccctgctaacacgcttctcctccgcccaggatctcggaactacctacagctgcgtcggtgtgatgcagaagggcaaggttgagattctcgtcaacgaccagggtaaccgaatcactccctcctacgtggcctttaccgacgaggagcgtctggttggcgattccgccaagaaccaggccgccgccaaccccaccaacaccgtctacgatgtcaagtcagttctaccgccctgttggcttctattgtataagtggacaattagctaactgttgtcacaggcgattgattggccgcaaattcgacgagaaggagatccaggccgacatcaagcacttcccctacaaggtcattgagaagaacggcaagcccgtcgtccaggtccaggtcaacggccagaagaagcagttcactcccgaggagatttctgccatgattcttggcaagatgaaggaggttgccgagtcgtacctgggcaagaaggttacccacgccgtcgtcaccgtccctgcctacttcaacgtgagtcttttccccgaaattcctcgaggattccaagagccatctgctaacagcccgataggacaaccagcgacaggccaccaaggacgccggtaccattgccggcttgaacgttctccgaatcgtcaacgaacccaccgctgccgctatcgcctatggtctggacaagaccgacggtgagcgccagatcattgtctacgatctcggtggtggtacctttgatgtttctctcctgtccattgacaatggcgtcttcgaggtcttggctaccgccggtgacacccaccttggtggtgaggactttgaccagcgcattatcaactacctggccaaggcctacaacaagaagaacaacgtcgacatctccaaggacctcaaggccatgggcaagctcaagcgtgaagccgaaaaggccaagcgtaccctctcttcccagatgagcactcgtatcgaaatcgaggccttcttcgagggcaacgacttctccgagactctcacccgggccaagttcgaggagctcaacatggacctcttcaagaagaccctgaagcctgtcgagcaggttctcaaggacgccaacgtcaagaagagcgaggttgacgacatcgttctggtcggcggttccacccgtatccccaaggttcagtctcttatcgaggagtactttaacggcaagaaggcttccaagggtatcaaccccgacgaggctgttgctttcggtgccgccgtccaggccggtgtcctttctggtgaggaaggtaccgatgacattgttctcatggacgtcaaccccctgactctcggtatcgagaccactggcggagtcatgaccaagctcattccccgcaacacccccatccccactcgcaagagccagatcttctcgactgctgccgataaccagcccgtcgtcctgatccaggtcttcgagggtgagcgttccatgaccaaggacaacaacctcctgggcaagttcgagcttaccggcattcctcctgccccccgcggtgtcccccagattgaggtttccttcgagttggatgccaacggtatcctcaaggtctccgctcacgacaagggcaccggcaagcaggagtccatcaccatcaccaacgacaagggccgtctcacccaggaggagattgaccgcatggttgccgaggccgagaagttcgccgaggaggacaaggctacccgtgagcgcatcgaggcccgtaacggtcttgagaactacgccttcagcctgaagaaccaggtcaatgacgaggagggcctcggcggcaagattgacgaggaggacaaggagactgtaagttgaagcgatccatcactgctttctgatgcggacatgtcacactaacacttgaccagattcttgacgccgtcaaggaggctaccgagtggctcgaggagaacggcgccgacgccactaccgaggactttgaggagcagaaggagaagctgtccaacgtcgcctaccccatcacctccaagatgtaccagggtgctggtggctccgaggacgatggcgacttccacgacgaattgtaa

SEQ ID NO: 2 - DNA, 1942 bp, Trichoderma reesei
  source 1..1942: mol_type = other DNA
atgaagttcaacaccgtcgcggccgctgcggctctgctcgctggtgtcgcgtatgccgaggacgtcgaggagtccaaggcagtcccggagcttcccacctttactgtgagtttgccctctctttcatctttggaaaaggacccaaatgttggcgcttggctccagcttggagcaagcttcttggacgacgggatatcatgaaccgctgctgacagttcccaccaatcgcttagcccacctccatcaaggcggacttcctcgagcagttcaccgacgactgggagtcccggtggaagccttcccacgccaagaaggacaccagcggctccgacaaggacgcagaggaggaatgggcctacgtcggcgagtgggcggtcgaggagccctaccagtacaagggcatcaacggcgacaagggcctcgttgtcaagaaccctgccgcgcaccacgccatctcggccaagttccccaagaagattgacaacaagggcaagacgctcgtcgtgcagtacgaggtgaagctccagagtaagtttggcctctgcaactcccccgtgataaccaaagcgagatgtggacattgtgctgacctatacgcttccagagggactggactgcggcggtgcctacatgaagctgctgcgcgacaacaaggctctccaccaggatgagttcagcaacaccaccccctacgtcatcatgtttggccccgacaagtgcggccacaacaaccgggtccacttcatcgtcaaccacaagaaccccaagactggcgagtacgaggagaagcacctcaactcggccccggccgtcaacattgtcaagacgacggagctctacaccctcattgtccaccccaacaacaccttctccatcaagcagaacggtgtcgagaccaaggccggcagccttctcgaggacctgagccctcccatcaaccctcccaaggagattgatgaccccaaggactccaagcccgacgactgggtcgacgaggctcgcattcccgaccccgaggccgtcaagcccgaggactgggacgaggatgcgccctttgagattgtcgacgaggaggccgtcaagcccgaggactggctcgaggacgagcccaccacgatccccgaccccgaggcccagaagcccgaggactgggatgacgaggaggacggcgactggatccctcccaccgtccccaaccccaagtgcgaggacgtctccggttgcggcccctggaccaagcccatggtcaggaaccccaactacaagggcaagtggactgctccttacattgacaaccctgcctacaagggcgtctgggctccccgcaagatcaagaaccccgactactttgaggacaagacgcccgccaactttgagcccatgggagctgtaagtttcgttcctttaccaagaccttcatgacgctcgattgctaaccagtgctcgacagattggcttcgagatctggaccatgaccaacgacatcctctttgacaacatctacattggccactccattgaggatgccgagaagctggccaacgagaccttcttcgtcaagcaccccattgagaaggcgcttgccgaggctgatgagcccaagtttgacgacacccccaagtcgccctctgacctcaagttcctcgacgaccccgtgacctttgtcaaggagaagcttgacctgttcctgaccattgcccagcgcgaccccgttgaggccatcaagtttgttcccgaggtcgccggtggcattgccgccgtcttcgtcaccctgattgccatcattgtcggtctggtcggccttggctcctcatcggccgcccccaagaaggccgccgccactgctaaggagaaggccaaggacgtttccgaggctgttgcaagcggtgccgacaaggtcaagggagaggttaccaagcgaaccacccgcagccagtcgtag

SEQ ID NO: 3 - DNA, 1910 bp, Trichoderma reesei
  source 1..1910: mol_type = other DNA
atgaagtcggcgagcaaattgttctttctctccgtgttttccctatgggcgacgccgggcgcatgctcaagctcgtcaagtacatgcactgtacgtcaacccaaccttggcctcgtttcccctttggaagaatgctttgcgctgacagattttgttgatctagttctccccaaacgccatcattgacgatggatgcgtttcgtatgcgactctcgatagactcaatgtcaaggtgaagcctgctatagacgaactcgttcagacgaccgacttcttttcgcactatcgcttgaacctcttcaacaaaaaatgccccttctggaacgacgaagatggcatgtgcggtaacattgcctgcgccgtcgagacgctggacaacgaagaagatattcccgagatatggagggctcacgagcttagcaagctggaaggccctcgagcgaagcatcccggcaagcaagagcagaggcagaaccctgagcgaccgctgcagggagagctgggggaggatgtaggggagagctgcgtggttgaatacgacgacgagtgtgacgacagagactactgcgtctgggacgacgaaggcgcaacgtccaagggggactacatcagcttgttgcgcaaccccgagcgcttcaccggctatggcggtcaaagtgcaaagcaggtgtgggacgccatctactcggagaactgcttcaagaagagctcgtttcccaagtcggccgatctaggcgtctcgcaccgcccaaccgaggcggctgctctggacttcaagcaggtcctggacaccgctggccgccaggctcaactggaacagcagcggcagagcaacccaaacattccctttgttgccaacactggctacgaggtggacgatgagtgtctggagaagcgcgtgttctaccgggtggtgtcgggaatgcacgccagcatcagcgtccacctgtgctgggacttcctgaaccagagcacggggcaatggcagcccaacttggactgctacgagagccgcctgcacaagtttccagaccgcatcagcaacctctacttcaactacgctctcgtgactcgcgccattgcgaagctgggcccgtatgtactgtcaccgcagtacaccttttgcacaggggacccgttgcaagaccaggagacgcgagacaagattgcggccgtcacgaagcacgcggctagcgtcccgcagatctttgacgagggcgtcatgtttgtcaacggcgaaggcccctcgctcaaggaagatttccgcaatcgcttccgcaacatcagccgggtcatggactgcgtcggctgcgacaagtgccgtctctggggcaagatccagaccagcggctacggcacggctttgaagattctgtttgagttcaacgagggccagaagccgccgcccctcaagaggaccgagctggtggccctcttcaacacgtatgccagactcagctcgtcggtggcggccgttgggcgattcagggccatgattgacatgcgcgacaagatggcgtccaagcccgacttcaagcccgaggatctctacacgctcatcgacgaggcggacgaggacatggacgagtttatcaggatgcaaaatcgtgggagccacggagatacgctgggcgagcaggtcggaaacgaatttgcccgcgtcatgatggccgtcaagattgtgctcaagagttggatccgaacgcccaagatgatgtaagtctcttctctcttttttttccccttcttcgagtggcacaaagctcttcattgagatggactaacacaattctagttggcaaattgtctcggaagagacgtcgagattgtatcgcgcttgggtcggtctgcctgcgcgacccagacggtacgcgttcagactgcccaacttgaatagagacgagttgtga

SEQ ID NO: 4 - DNA, 3027 bp, Trichoderma reesei
  source 1..3027: mol_type = other DNA
atgaagtcaccgaggaaatcaccgttgctgaagctcctcggagccgcctttctcttctccaccaacgttctcgccatctccgctgttctcggagtcgatctgggaaccgagtacatcaaggcggcgctggtgaagcccggcatcccgcttgagattgtgctcacgaaagattcccgacgaaaagaaacctcggccgtcgccttcaagccggcaaagggcgccttaccggagggccagtaccccgaacggagctatggcgccgacgcaatggcactcgccgcacgattccccggcgaagtatacccgaatctgaagcccctgcttggactgccagtgggggatgccattgtccaagaatatgcggccaggcaccctgcgttgaagctacaggcgcaccccacgcggggaactgctgcgttcaagacggagacgctgtctccggaagaggaggcttggatggtggaggagctgttggccatggagcttcagagcatccagaagaacgcagaggttaccgctggcggcgactcttcgatacgctccatcgtgctcaccgtcccgccgttttacaccatcgaggagaagcgagccctgcagatggcagcagagctcgccggcttcaaggtcctgagccttgtcagcgacggactggccgtgggcctcaactatgccaccagtcgccaattcccgaatatcaacgaaggcgccaagccggaataccacttggtctttgacatgggagcgggctccacaactgctacggtcatgaggttccaaagccgtacggttaaggacgtcggcaagttcaacaagacggttcaggagatccaggttctcggcagcggctgggacaggaccctcggaggagactctctcaactcgctaatcatcgatgacatgattgctcagtttgtggaatccaagggtgctcagaagatttcggcaaccgccgagcaggttcagtctcatggccgcgccgttgcaaagctgagcaaggaagccgagcgtctccgacacgtcctcagcgccaaccagaacacccaagccagctttgagggactgtacgaagatgttgacttcaagtacaagatctctcgggctgacttcgagaccatggcaaaggctcatgtcgagcgagtcaacgctgccatcaaggacgctctgaaggccgcgaacctcgagattggcgatctgacttccgtcattcttcacggtggtgcgacccgtactccgtttgtgcgagaggccattgagaaagctcttggttctggcgacaagatccgtaccaatgtcaactctgatgaggcagccgtctttggtgctgctttccgggctgctgagctcagcccaagcttccgtgtgaaggagattaggatttctgagggtgcaaactacgcagctggcattacttggaaggctgcgaacggcaaggtacaccgccaacgactctggactgccccgtcgccgctcggtggcccggccaaggagattacctttacggaacaggaggactttactggtttattctatcaacaagttgacactgaggataagcccgtcaagtcgttctcgactaagaaccttaccgcctctgttgctgctctgaaagaaaagtatcccacttgtgccgatactggcgttcagttcaaggctgccgcgaagctccgtaccgagaacggcgaggttgccatcgtcaaggcctttgtggagtgcgaggctgaagtcgttgagaaggaaggctttgttgacggcgttaagaacctctttggcttcgggaagaaagatcagaagcccctcgccgaaggaggagacaaggacagtgccgatgcgtctgcggattctgaggccgagacggaggaagctagctctgcgacaaagtcctcctcttccaccagcaccaccaagtccggagatgctgccgagtcaacagaggctgcaaaggaagtcaagaagaagcagcttgtttctatccctgtcgaagtcacgttggaaaaggctggaatccctcagcttaccaaggccgagtggaccaaggccaaggatcgactgaaggcattcgccgcctccgacaaggccaggctgcagcgcgaagaggccctgaaccagctcgaagcattcacttacaaggttcgcgaccttgtcgacaacgaagccttcatctccgcgtctaccgaggcggagcgacagacgctctctgaaaaggctagcgaagcaagtgactggctttatgaggagggcgactcggccacgaaagatgactttgttgctaagctcaaggctctgcaagatctcgtggcaccgatccagaaccgcctggacgaggctgagaagcggcctggtctgattagcgatctgagaaacattctcaacaccacaaatgtgtttattgacactgttcgtgggcagattgctgcgtatgatgaatggaaatccacagcttcagccaagtcggctgaatcagccacctcgagtgctgccgccgaggcgacgaccaacgactttgaagggctcgaggatgaggacgacagccccaaagaggctgaggagaagcccgttccagaaaaggtcgtgcccccgctgcacaactctgaggagattgacacgctcgaggttctctacaaggagactctggagtggctgaacaagctcgaacgccaacaggcagatgttcctctcaccgaagagcccgtgcttgttgtcagcgagctggttgccagacgagatgcgcttgacaaggccagcttagacctcgcgctgaagagctacacccaataccagaagaacaagcccaagaagcccaccaagagcaagaaggcgaagaagcaggacaagacgaagagcgccgacaaggctggcccgacgtttgagtttcccgagggcagcgtgcccctctccggcgaggagctggaggagctggtcaagaagtacatgaaggaggaggaggagacccgcaggcaggccgagggcggacaggcagaggagaagccggcggaagatacagagaagtcgagccatgacgagctctaa

SEQ ID NO: 5 - DNA, 1417 bp, Trichoderma reesei
  source 1..1417: mol_type = other DNA
atggtagccagattgtccagcatctacgcctgtgggctcttagcctggacgcacattgtttgcgcctctcagtttagcgacccgatgcaactacagaagcatcttgcacagaatgactatactttaattgcttgtaagtcatgacaatatcgccttctaaagtgtgtcaactcaggtagaaataattgctaatagtagcttacagttgttgctgtaagagttgttcgggtcaatatcttacctagtcaagtcgagactcgaggctgaccttaaggtatcgctaccacttacagcctcaactgtaagttttggccaggccaagtttgaacccatctcccttaagaacaccgaacttaaaaaaagtcaaacggcagagaagccagcaaactcctcttagaagaatggcagacggtccagcaacatgtcgcctccaccgccaccatcgactgtccgtccagccctaaactctgtcaggagatggacgtcgcctcctttcccgctattcggctctaccgccaggatggctcagtaacacgttatcgagggcctcgtcggaccgcaccgtgagttgacactttcttcgaattttggagttaatctctcaaagcatgaagtgactgactgactaccttacctcccaggatcgacgcctttgtgaagcgtgctctcaaaccatccgtgcagaatgttcctgggcagcaacttgccaacttcatcaccaacgacgactatgtattcatcgccaagctgcaaggcgagagcgagagcatcaattctcactacagggattttgcgcaagagtattctgatcgatactcgtttggcatcatcacgagtggctctgtaccctccaatggcgtctggtgctacaacaacgtcgacggaaatcagcacgcggcgacggacttgaacgatccaaatgccttgaagaagcttctcaatctttgcaccgcggaggtcattccccagcttacacgacgcaatgagatgacttatctttccgtatgtcttctgttctccctcctcacttttaaaatgttcagtagaagaagcttgggcttctgaccccttattccagtcaggccgatccttggtctattacttctccaacaatgaagcagaccgcgaagcatacgtcaaagcgctcaaacccatcgcccagcgatacgccgagttcctccagttcgtcaccgtcgactctggcgagtatcccgatatgctgcgcaatctgggcgttcgctccgccggaggcctggcagtgcaaaacgtccacaacggacatattttccccttcagaggagacgctgctgcttcgcctggacaggttgaccagttcattgtggccatctcagaaggtagggcgcagccttgggatgggaggtttgacgagggacaggaggcgcatgatgagctctga

SEQ ID NO: 6 - DNA, 2174 bp, Trichoderma reesei
  source 1..2174: mol_type = other DNA
atgcggctaacatccttcttctctggcctggccgcctttggccttctgtcatctccagcactggcagatgatgaagctgacaacgtccccgcgcccacatacttcgattccgtcatggtgcctcccttgacagaactaacgccagacaacttcgaaaaggaggcaagcaaaaccaagtggcttcttgtgaagcactacaggtactaagcccttcagccatatcacaccactccccgtctgattcaagctgacgcgtagccgctgtctagtccatactgccaccattgtatcagctacgccccgaccttccagacaacctacgaattctactacacatccaagccagaaggagctggcgacacgagcttcaccgacttctacgacttcaagtttgctgccgtgaactgtatcgcctacagcgacctttgcgttgagaatggcgtcaagctataccccactacggttctatacgagaacggcaaagaggtcaaggccgtaacgggtggccagaacatcaccttcctttctgatctcatcgaagaagctttggagaagtcgaagcctggatctcggcccaagtctctcgcattgccccaaccgggcgacaaagagcgccccaaatctgagcccgagacagcatcgaggagcgcaaccgaggagaagaagcccaagaagccggttgccacgccgaacgaagacggagtgtcagtttccttgacggccgaaaacttccagcgcctggtgactatgactcaggatccctggttcatcaagttttacgcgccgtggtgcccccattgccaagacatggcgcctacctgggagcagctggcgaagaacatgaagggcaagctcaacattggagaggtcaactgtgacaaggagtcgcgattgtgcaaagacgttggtgcgcgggcgtttcccactatcctgttcttcaagggtggagagcgctcagagtacgaggggctccgaggcctgggcgactttatcaaatatgccgaaaacgccgtcgacctcgctagcggagtgcctgacgtggacttggcagcattcaaggctctcgagcagaaggaagacgtcatctttgtctacttttacgaccacgccaccacatcggaggacttcaatgccctcgagaggctgcccctgagtctcatcggacatgccaaactggttaagactaaggatccggccatgtacgagcgcttcaagatcacgacatggcccagattcatggtttcgagggagggtcgccctacgtactaccctcccctcacccctaacgcgatgagagatacccaccaagttctggactggatgaggtcggtttggcttccccttgtccccgaactgttggttaccaacgcccgccagatcatggacaacaaaattgttgtgctcggcgtcctgaatcgagaagaccaggaatccttccagagtgctcttcgggagatgaagagcgcagccaacgagtggatggacaggcaaatccaagagttccagttggagcggaagaagctgcgagacgcgaagcaaatgaggatcgaggaagctgaggaccgagacgatgagcgcgccctgcgggccgccaaggcgatccatattgacatgaacaattccggacggagagaagtggcctttgcgtgggttgatggcgtagcgtggcagcgctggattcgaaccacgtatggcattgatgttaaggacggagaaagagtcattatcaacgaccaagatgtaagcctcaagctcacccccatttgtcctccctctacaatattgctttgcgtttcgaacatgaacgactaacaaaaacatttgaacagagccgcaagtactgggacagcaccgtgacgggcaactacatcctcgtcagccgcacgtccatcctggagacgctcgacaaggtcgtctacaccccgcaggccctcaagcccaagctcaccatttcctctttcgagaagatctttttcgacatccgcgtctccttcaccgagcacccctacctgaccctgggctgcatcgttggcatcgcctttggagccttctcctggctgcgtggccgctctcgccgtggacgcggccacttccggctcgaggattccatcagcattagagatttcaaggacgggttccttggtggatctaacggcaacaccaaggccgactga

SEQ ID NO: 7 - DNA, 1578 bp, Trichoderma reesei
  source 1..1578: mol_type = other DNA
atgcatcagcaaaccctcctcgccaccctcgcggcgagtctcgctgctcttccttttgctcaggcgggcttctattcgaagagctctcccgtgctgcaagtagacgccaagtcgtacgaccgcctcatcacaaagtcgaatcatacctctgtaagtatccgtcctcacacactcacctcactcacaacgcgacatcatatctcatacacatccaccccaaaccaccacaaacacaagacatatatcaagctcaaacacatacacatacatacaaacacatacacacacagatacatacacaactctcatatatatgaaccattcattgacatttcccccaagattgtcgaattctacgccccctggtgcggccactgccaaaacctcaagcccgcctacgaaaaggccgcccgcaccctcgacggcctggccaaggtcgccgccgtcgactgcgacgacgacgccaacaaggccctctgcggctccctcggcgtcaagggcttccccaccctcaagatcgtccgccccggcaagaagcccggccgccccgtcgtcgaggactaccagggccagcgcaccgcgggcgccattgccgacgccgtcgtcgccaagatcaacaaccacgtcgtcaagctgacggacaaggacattgatgcctttctggaaaaggacggcgacaagccaaaggccatcttgttcacggaaaagggaactacgagtgcgctgctgaggagccttgctattgattttctcgacgccgtgaccattggccaggtccgcaacaaggaaaaggctgccgtcgacaggttcggcatctcttcgttcccttccttcgtcctcatccccggaggcggcaaggagcccgtcgtctacagcggcgagctcaacaagaaggacatggtcgagttcctcaagcaggtcgccgagcccaaccccgacccggccccctcaaacggcaagtccggcaagaaggcctccaccaaggacaaggccagcagcaaggaggccccccaaaaggccgccgccgccgacgagtcttcgtccgccgcatcctccgagacctcaacggccgccgcgccggagtcgaccctcatcgacatccccgccctgacttccaaggcagagctcgaggagcactgtctccaaccaaagtcccaaacctgcgtcctcgcctttgtgcccgcgtccgcctcggagatgcgcaacaagatcctttctgccgtctcccagctgcacaccaagtacgtccacggaaagcgccacttccccttcttctctgtcgacagcgacgtcgaaggctctgccgccctcaaggaagccctcggcctctcgggcaagattgagctcgttgccctcaacgcccgccgggggtggtggaggcgatacgaggacggtgagttcagcgttcacagcgtcgagtcctggattgacgccgttcgcatgggcgagggcgagaagaagaagcttcccgagggagtcgtcgtcgagaaggcggagccggcggaggaagcaaagtctgagactgaagctgccgcagctgatgaggccactgagaagcctgagcacgatgagctctaa

SEQ ID NO: 8 - DNA, 1167 bp, Trichoderma reesei
  source 1..1167: mol_type = other DNA
atggtcttgatcaagagcctcgtgctcgccgtcctggccagctcggtggctgccaagtcggccgtcatcgacctgattccgtccaactttgacaagcttgtcttctccggaaagcccacgcttgtcgagttttttgctccctggtgcggccactgcaagaaccttgctcccgtgtacgaggagttggcccaggtgtttgagcatgctaaggacaaggtccagattgcaaaggtcgacgccgactcggagcgagacctcggaaagcggttcggcatccagggcttccccacgctcaagttcttcgatggcaagagcaaggagccgcaggagtacaagtcgggccgtgatctggacagcctgaccaagttcatcactgagaagactggtgtcaagcccaagaagaagggcgagctgcccagcagcgtggtgatgctgaacactaggaccttccacgacactgttggaggcgacaagaatgtcctggtagcgttcactgctccttggtgtggccgtaagtgaagcctcgacccccgactgagtcttgattctcgcatatttacctcttgaccagactgcaagaacctcgcccccacttgggaaaaggttgccaatgacttcgcgggtgatgagaacgttgtgattgccaaggtcgatgccgagggcgctgacagcaaggccgtcgccgaagagtacggcgtcactggctaccccaccatcctcttcttccccgctggcaccaagaagcaggttgactaccaaggcggccgatcggagggtgactttgtcaacttcatcaacgagaaggccggcaccttccgaaccgagggcggcgagctgaatgacatcgccggcaccgtggcgcccctcgacaccatcgtggccaacttcctcagcggcaccggcttggccgaggctgctgctgagatcaaggaggctgttgacctgcttacggatgctgcggagaccaagttcgccgagtactacgtccgcgtcttcgacaagctgagcaagaatgagaagtttgttaacaaggagcttgcgagactgcagggcatcctggccaagggtggccttgccccttctaagcgggatgagatccagatcaagatcaacgtcctgcgcaaatttacccccaaggagaacgaggaccagaaggacgagctgtga

SEQ ID NO: 9 - DNA, 1705 bp, Trichoderma reesei
  source 1..1705: mol_type = other DNA
atgcaacagaagcgtcttactgctgccctggtggccgctttggccgctgtggtctctgccgagtcggatgtcaagtccttgaccaaggacaccttcaacgacttcatcaactccaatgacctcgtcctggctgagtgtatgtctctctctctctctctccccccctcccctttgccttctgccctctcaagcttctgcatctctcgacccctcccccgccagccccccggcatcgagatccccgctaacagctgcaatcttccagtcttcgctccctggtgcggccactgcaaggctctcgcccccgagtacgaggaggcggccacgactctcaaggacaagagcatcaagctcgccaaggtcgactgtgtcgaggaggctgacctctgcaaggagcatggagttgagggctaccccacgctcaaggtcttccgtggcctcgataaggtcgctccctacactggtccccgcaaggctgacgggtaagctttgaattgcactgttctttgcatcaatccattcattcgctaacgttggttgtcctttcagcatcacctcctacatggtgaagcagtccctgcctgccgtctccgccctcaccaaggataccctcgaggacttcaagaccgccgacaaggtcgtcctggtcgcctacatcgccgccgatgacaaggcctccaacgagaccttcactgctctggccaacgagctgcgtgacacctacctctttggtggcgtcaacgatgctgccgttgctgaggctgagggcgtcaagttcccttccattgtcctctacaagtccttcgacgagggcaagaacgtcttcagcgagaagttcgatgctgaggccattcgcaactttgctcaggttgccgccactcccctcgttggcgaagttggccctgagacctacgccggctacatgtctgccggtatccctctggcttacatcttcgccgagaccgccgaggagcgtgagaacctggccaagaccctcaagcccgtcgccgagaagtacaagggcaagatcaacttcgccaccatcgacgccaagaactttggctcgcacgccggcaacatcaacctcaagaccgacaagttccccgcctttgccattcacgacattgagaagaacctcaagttcccctttgaccagtccaaggagatcaccgagaaggacattgccgcctttgtcgacggcttctcctctggcaagattgaggccagcatcaagtccgagcccatccccgagacccaggagggccccgtcaccgttgtcgttgcccactcttacaaggacattgtccttgacgacaagaaggacgtcctgattgagttctacgctccctggtgcggtcactgcaaggctctcgcccccaagtacgatgagctcgccagcctgtatgccaagagcgacttcaaggacaaggttgtcatcgccaaggttgatgccactgccaacgacgtccccgacgagatccagggcttccccaccatcaagctctaccccgccggtgacaagaagaaccccgtcacctacagcggtgcccgcactgttgaggacttcatcgagttcatcaaggagaacggcaagtacaaggccggcgtcgagatccccgccgagcccaccgaggaggctgaggcttccgagtccaaggcctctgaggaggccaaggcttccgaggagactcacgatgagctgtaa

SEQ ID NO: 10 - DNA, 982 bp, Trichoderma reesei
  source 1..982: mol_type = other DNA
atgaaggcagccctgctcctctccgccctggcctcgtgcgccattggcctcgtcgccgccgccgccgaggacttcaagatcgaggtcacccaccccgtcgagtgcgaccgcaagacgcaaaagggcgacaagctgtccatgcactaccgcggcacgctggccaagacgggcgacaagttcgatgccagtgcgtttcttctattccctttccctctttcctcccatttctctcacacaccaatgacggtcctccttttcttttgatctcattgactgacaagttttggtctacctactctaggctacgatcgtaaccagccattcaacttcaagctgggtgctggccaggtgattaaggggttcgtcttgcccacccccccctaacccacccctctcgttcttttatgacgacgacgacgacgacgacgttgggcgacgttgaggctaacggcttgtagatgggatcagggtctccttgacatgtgcattggcgagaagaggtaagacgaaccgaaccaacccaactgcgtcgctcactgcctccttgggcctctatcaggacgcaatgctgaccattacatcaccaattcaggactctcacgatccctcccgagctgggctacggccagcgcaacatgggccccattcccgccggctcaaccctgagtacgtggctcctatcctcccctacctgaactcccaaacccagagtttcacccacgccgcatggaaaaccaggccgcaggctaacaacacacgatgccatacagtctttgagaccgagctcctcgccatcgagggcgtcaaggcccccgagaagaagcccgtccccgagacgcccattgtcgagaagcccgccgaagagacagaggagagcgtcgtcgagaaggccgccgaggcagccgccagcgtggcctccgaggccgtcgacgccgccaagactgtctttgccgacactgacgagggtcacggggagctgtaa

SEQ ID NO: 11 - DNA, 809 bp, Trichoderma reesei
  source 1..809: mol_type = other DNA
atgctgacctttaggcggctcttcaccaccgccatcgtcctggtggtgggcctgctcttcttcgtcaagacggccgaggccgccaagggccccaagatcacccacaaggtcttcttcgacattgagcacggcgacgagaagctgggccgcatcgtcctgggcctgtacggcaagacggtccccgagacggccgagaacttccgggccctggccaccggcgagaagggcttcggctacgagggctcgaccttccaccgcgtcatcaagcagtttatgattcagggcggcgactttaccaagggcgatggcaccggtggcaagtcgagtaagttgcctttggttcccaaataagcaatcaattgatcaatcaattgggtggcatggcgtttgtcactgcatctggctctggctctggctaaccttgagggctccgtctagtctacggcaacaagttcaaggacgagaacttcaagctgaagcacaccaagaagggcctgctgtccatggccaacgcgggacccgacaccaacggctcccagttcttcatcaccactgttgttacctcgtatgatttccccaccctccttggaagatcctggataagaagtaggaccaatctaacgaacaacttaaacagatggctcgacggccgacacgtcgtcttcggcgaggttctcgagggctacgacattgttgagaagattgaaaacgtccagaccggccccggcgatcgcccagtgaagccggtcaagattgccaagagcggcgagctggaggttccccccgaaggtattcacgtcgagctctaa

SEQ ID NO: 12 - DNA, 1372 bp, Trichoderma reesei
  source 1..1372: mol_type = other DNA
atgatactgcgcgcggcaatcttcgtcttgctggcgctggtatcgctggcggtttgcgccgaggacttttacaaggtatgccgggacgcaatgcctcgaatcaagcacggagcgtgctgacggacacatgacaggttctaggagtcgacaagtctgcgtcagacaagcagctcaagcaggcctatcgccagctctccaagaagttccacccagacaagaacccgtacgccctcctacagctacacgcagtctcgccaaccttctccaatgtgctaatcactctactgcttctagaggcgatgaaacggcgcacgagaaattcgtgctggtgtccgaggcctacgaagttctgagcgattccgagcttcgcaaagtctacgaccgctacggccacgagggcgtcaagtcccaccgtcaaggcggcggcggaggaggaggaggcgaccccttcgacctcttcagcaggttctttggcggccatggccactttgggagaaacagccgcgagccccggggcagcaacattgaggtccgcatcgagatttccctccgcgacttttacaacggcgccacgaccgagttccagtgggagaagcagcacatatgcgaaaagtgcgagggcacgggcagcgcggacggaaaggtcgagacgtgcagcgtctgcggcggacacggggttcggattgtcaagcagcagctcgttcccggcatgttccagcagatgcagatgcgctgcgaccactgtggcggctcgggcaagaccatcaagaacaagtgttccgtctgccacggcagccgagtcgagcgcaagccgacgactgtcagcctgactgtcgagaggggcattgctcgagatgccaaggtggtgtttgagaacgaagccgaccagagccccgactgggttcctggtgatctcattgtcaacctgggcgagaaggccccgtcatacgaagacaaccccgatcgcgtcgacggcaccttcttccggcgcaagggccatgacctgtactggaccgaggttctgtcgctgcgtgaggcctggatgggtggctggacgcgtaacctcacgcacctcgacaagcacgttgtgcgtcttggacgggagcgaggccaggttgttcagagtgggttggtggaaaccattcccggcgaaggcatgcccatatggcacgaagagggagagagcgtctatcacacacacgagtttggaaatctctacgtcacatacgaagtcattttgccggaccagatggacaagaagatggagagcgagttctgggacctgtgggagaagtggcggtccaagaatggtgtggacctgcaaaaggatctcgggcggcctgagccagggcatgaccatgatgagttatga

SEQ ID NO: 13 - DNA, 685 bp, Trichoderma reesei
  source 1..685: mol_type = other DNA
atggcgcgccgccagcacctcaccgcgacagtcctgctggccgtcgtgctcttcttcagcatcacgtacctcctctcgggctcgtccagctccaatgcggatcgaacgcgcgaggccgtagtggcagagcccaagtcggaattcaaggtggattttgacggcatgccggccaacctgctggagggagagtcaatagcacccaagctggagaatgcgactctcaagtacgtttcccgcatacccgaacctgctcccatgagccaccgaccatggcagtgtttcaaaggataccagttctgacgcttttctgcaattacatagagccgagctcggtcgcgcaacatggaaattcatgcacacaatggtcgcccgcttccccgagaagccctcgcccgaggagcgcaagacgctcgagaccttcatctacctcttcggccggctgtacccctgcggcgactgcgcgaggcacttccggggcctgctggcaaaatatccgccgcagacgagtagccggaatgcggctgccggatggctgtgttttgtgcacaaccaggtcaacgagaggctgaagaagcccatatttgactgcaacaacattggcgacttttacgactgcggctgcggggacgagaagaaggacgggaaggaggaggccaaggttgatggcgaattggtgaaggaatag

SEQ ID NO: 14 - DNA, 3407 bp, Trichoderma reesei
  source 1..3407: mol_type = other DNA
atggtgatgctggtggcgatcgcgctcgcatggctgggatgctcgctgctgcggccggtagatgccatgcgcgcagactatctggcccagctgcggcaggagacggtggacatgttctatcacggatatagcaactacatggagcatgcgtttcccgaagacgaggtgggttccgctgcgatagaagattgttgttggggctgctgctatgttccagctcccggggggtcggattctctcatatagaactagacagctaacgacttgtgccttttccatatgcttagctgcgtcccatatcgtgcactcccctgacgcgagatcgagacaatccggggcgcatcagcctcaacgatgccctcggcaactactctctgaccctcatagacagcctgtctacccttgccatcctggccggcggcccgcagaacggcccttacacgggaccgcaggctctgagcgacttccaggatggcgtggccgagtttgtgcgacactacggagacgggcgatcggggccctccggcgctgggatacgtgccagaggctttgatctcgacagcaaagttcaggtctttgagaccgtcatccggggcgtgggcggtctccttagcgcgcacctgttcgccattggggagctgccgattaccggatacgtgcccaggccggagggagtcgcaggcgatgatcctctggagctggcccctattccgtggcccaatgggttcaggtacgatggccagctgctgaggctcgcgctcgacctctccgagaggctgcttcccgccttctacacgccgacgggcattccgtatcctcgtgtcaatctccgcagcggcatccccttttacgtcaactcgcctctccaccaaaacctgggcgaggcagtggaggagcagagtggccgtcctgaaattaccgagacctgcagcgccggggcgggaagcctggttctcgaatttaccgtcttgagcaggctcacgggagacgccaggtttgaacaagccgccaagcgagcattctgggaggtctggcatcgcaggagcgaaattggcttgatcgggaacggcatcgacgccgagcgcgggctgtggatcggccctcacgcgggcattggcgcgggcatggacagcttctttgaatatgcgctcaagagccatatcctcctctcgggcctcggtatgcccaacgcctccacgtcgcgccgacagagcacaaccagctggctggatccaaactccctgcacccgccgctgccaccagagatgcacacgtcagatgccttcctccaggcatggcatcaggcgcacgcctcggtcaagcggtacctgtacaccgaccggagccacttcccttattactccaacaaccaccgtgccacgggccagccctatgccatgtggatcgacagcctgggcgccttctatccggggctcctcgccctggccggtgaggtggaagaggccattgaggcgaacctcgtctacacagccttgtggacgcggtactctgcgctgcccgaacgctggtccgtccgcgaaggcaacgtcgaggcaggcatcggctggtggcccgggaggcccgagttcatcgagtcgacgtaccacatctaccgtgcaacccgcgacccgtggtatctgcacgttggcgagatggtcctccgcgacattcggcgtcggtgctatgcggagtgcggctgggccgggcttcaggacgtgcagacgggcgagaagcaggaccgcatggagagcttcttcttgggagagacggcaaaatacatgtacctgctgttcgacccagaccatccactcaacaagctggatgccgcctacgtcttcaccacagaaggccatccgcttatcataccaaagagcaaaaggggtagcggctctcacaacagacaggaccgcgctcgcaaagccaagaagagccgagacgtcgcagtctacacctactacgatgaaagcttcacaaactcttgtccggcccctcggccgccttcagagcatcacctgataggctcggccacggcggccaggccagacttgttctccgtctctcgcttcacagacctgtacagaacgcccaacgtacacgggcccctggagaaggtggagatgcgagacaagaagaagggccgggtggttcgatacagggccacctcaaaccacaccatcttcccctggactcttcccccagccatgctgccggagaatggcacctgcgctgctcccccggaacgcatcatatccttgattgagttcccggccaacgacatcaccagtggaatcacgtcgcggttcggcaaccatctatcgtggcagacgcatctggggccaacggtcaacattctagagggactgaggctccagctcgagcaggtgtcggaccctgccacgggagaagacaagtggaggatcacacacattggcaacacgcagctggggcgccacgagacagtcttcttccacgcggaacacgtaaggcatctcaaggacgaggtgttttcctgccgcagaaggagggacgccgtggaaatcgagctcctggtcgacaagccgagcgataccaacaacaacaacacgcttgcctcgtccgatgacgatgtagtggtagatgcaaaagcagaagagcaagacggcatgctagccgacgacgacggcgacacactcaacgcagaaacactctcctccaactccctcttccagtccctcctccgcgccgtctcctccgtcttcgagcccgtctacaccgccatccccgagtccgaccccagcgccggcaccgccaaggtctacagtttcgacgcctacacgtccaccggccccggcgcgtaccccatgccgtccatctcggacacgcccatccccggcaaccccttttacaacttccgcaacccggcctccaacttcccctggtcgaccgtcttcctcgccggccaggcctgcgagggcccgctccccgcgtccgcgccgcgcgagcaccaggtcattgtcatgctccgcggcggctgctccttcagccgcaagctggacaacatccccagcttctcgccccacgacagggcgctgcagctcgtcgttgtcctcgacgaaccgccgccgccgccgccgccgccgccagccagtcagaacagcggcggcgatgacgacgatgaagatgacgaagacgaccacgacgccgtcaacgacaacgaagacgacaggcgcgacgtgacgcggccactgctcgacacggagcagaccacgcccaagggcatgaagcgcctgcacggcatcccaatggtcctcgtccgagccgcgcggggcgactacgagcttttcgggcatgccattggcgtgggcatgaggcgcaagtatcgggttgaaagccaggggcttgtcgtggagaatgcggttgtgctgtga

SEQ ID NO: 15 - DNA, 1221 bp, Trichoderma reesei
  source 1..1221: mol_type = other DNA
atgaggcctctggcactcatatttgccctcatcttgggcctattgctctgcttagcagccccagcaacggcatcgtcatcatcatcacaacactctccccaagcggcatcagacgagtcagatttaatatgtcacacatcaaacccagacgaatgctatccccgggtcttcgtaccaacgcatgagttccagccagtccacgacgaccagcaactcccaaacggcctccatgtccgtctcaacatctggaccggccaaaaggaagccaagatcaacgtccccgatgaggccaaccctgatctcgatggcctgcccgtcgaccaagccgtggttctcgtcgaccaggagcagccagaaattatccagatccccaagggcgcaccaaaatacgacaatgtcggcaagatcaaggaacccgcgcaagaaggagacgcccaaacggaagccattgcttttgcagagacgttcaacatgctcaagaccggcaagtcgccaagcgccgaggagttcgacaacggactggaaggcctggaggagctctcccacgacatctactacgggctcaaaatcacagaggacgcggacgtggtcaaggcgctattctgcttgatgggggctcgcgacggcgacgcctcggagggagccacgccgcgcgaccagcaagcggccgcgatcctcgccggcgccctgtccaacaatccgtcggcactcgccgagatagccaagatctggcctgagcttctggactcgtcgtgtcctcgcgacggcgccaccatctctgaccgtttctaccaagacaccgtctccgttgccgactctccggcaaaggtcaaggccgccgtctcggccatcaacggcctgatcaaggacggcgccatccgaaagcagtttctcgaaaacagcggcatgaagcagctcctctcggtcctgtgccaagagaagccggagtgggcgggagcgcagcggaaagtcgctcagctggtgctggacaccttcctggacgaggacatgggcgcccagcttggccagtggcccaggggcaaggcatcgaacaacggggtgtgtgcggcgccggagacggcgctcgatgacggatgctgggactatcatgcggacaggatggtgaagctgcatgggacgccgtggagcaaggagttgaagcagaggctgggagatgcgcgcaaggcgaacagcaagttgccggatcatggcgagctgtag

SEQ ID NO: 16 - protein, 664 aa, Trichoderma reesei
MARSRSSLALGLGLLCWITLLFAPLAFVGKANAASDDADNYGTVIGIDLGTTYSCVGVMQKGKVEILVNDQGNRITPSYVAFTDEERLVGDSAKNQAAANPTNTVYDVKRLIGRKFDEKEIQADIKHFPYKVIEKNGKPVVQVQVNGQKKQFTPEEISAMILGKMKEVAESYLGKKVTHAVVTVPAYFNDNQRQATKDAGTIAGLNVLRIVNEPTAAAIAYGLDKTDGERQIIVYDLGGGTFDVSLLSIDNGVFEVLATAGDTHLGGEDFDQRIINYLAKAYNKKNNVDISKDLKAMGKLKREAEKAKRTLSSQMSTRIEIEAFFEGNDFSETLTRAKFEELNMDLFKKTLKPVEQVLKDANVKKSEVDDIVLVGGSTRIPKVQSLIEEYFNGKKASKGINPDEAVAFGAAVQAGVLSGEEGTDDIVLMDVNPLTLGIETTGGVMTKLIPRNTPIPTRKSQIFSTAADNQPVVLIQVFEGERSMTKDNNLLGKFELTGIPPAPRGVPQIEVSFELDANGILKVSAHDKGTGKQESITITNDKGRLTQEEIDRMVAEAEKFAEEDKATRERIEARNGLENYAFSLKNQVNDEEGLGGKIDEEDKETILDAVKEATEWLEENGADATTEDFEEQKEKLSNVAYPITSKMYQGAGGSEDDGDFHDEL

SEQ ID NO: 17 - protein, 558 aa, Trichoderma reesei
MKFNTVAAAAALLAGVAYAEDVEESKAVPELPTFTPTSIKADFLEQFTDDWESRWKPSHAKKDTSGSDKDAEEEWAYVGEWAVEEPYQYKGINGDKGLVVKNPAAHHAISAKFPKKIDNKGKTLVVQYEVKLQKGLDCGGAYMKLLRDNKALHQDEFSNTTPYVIMFGPDKCGHNNRVHFIVNHKNPKTGEYEEKHLNSAPAVNIVKTTELYTLIVHPNNTFSIKQNGVETKAGSLLEDLSPPINPPKEIDDPKDSKPDDWVDEARIPDPEAVKPEDWDEDAPFEIVDEEAVKPEDWLEDEPTTIPDPEAQKPEDWDDEEDGDWIPPTVPNPKCEDVSGCGPWTKPMVRNPNYKGKWTAPYIDNPAYKGVWAPRKIKNPDYFEDKTPANFEPMGAIGFEIWTMTNDILFDNIYIGHSIEDAEKLANETFFVKHPIEKALAEADEPKFDDTPKSPSDLKFLDDPVTFVKEKLDLFLTIAQRDPVEAIKFVPEVAGGIAAVFVTLIAIIVGLVGLGSSSAAPKKAAATAKEKAKDVSEAVASGADKVKGEVTKRTTRSQS

SEQ ID NO: 18 - protein, 585 aa, Trichoderma reesei
MKSASKLFFLSVFSLWATPGACSSSSSTCTFSPNAIIDDGCVSYATLDRLNVKVKPAIDELVQTTDFFSHYRLNLFNKKCPFWNDEDGMCGNIACAVETLDNEEDIPEIWRAHELSKLEGPRAKHPGKQEQRQNPERPLQGELGEDVGESCVVEYDDECDDRDYCVWDDEGATSKGDYISLLRNPERFTGYGGQSAKQVWDAIYSENCFKKSSFPKSADLGVSHRPTEAAALDFKQVLDTAGRQAQLEQQRQSNPNIPFVANTGYEVDDECLEKRVFYRVVSGMHASISVHLCWDFLNQSTGQWQPNLDCYESRLHKFPDRISNLYFNYALVTRAIAKLGPYVLSPQYTFCTGDPLQDQETRDKIAAVTKHAASVPQIFDEGVMFVNGEGPSLKEDFRNRFRNISRVMDCVGCDKCRLWGKIQTSGYGTALKILFEFNEGQKPPPLKRTELVALFNTYARLSSSVAAVGRFRAMIDMRDKMASKPDFKPEDLYTLIDEADEDMDEFIRMQNRGSHGDTLGEQVGNEFARVMMAVKIVLKSWIRTPKMIWQIVSEETSRLYRAWVGLPARPRRYAFRLPNLNRDEL

SEQ ID NO: 19 - protein, 1008 aa, Trichoderma reesei
MKSPRKSPLLKLLGAAFLFSTNVLAISAVLGVDLGTEYIKAALVKPGIPLEIVLTKDSRRKETSAVAFKPAKGALPEGQYPERSYGADAMALAARFPGEVYPNLKPLLGLPVGDAIVQEYAARHPALKLQAHPTRGTAAFKTETLSPEEEAWMVEELLAMELQSIQKNAEVTAGGDSSIRSIVLTVPPFYTIEEKRALQMAAELAGFKVLSLVSDGLAVGLNYATSRQFPNINEGAKPEYHLVFDMGAGSTTATVMRFQSRTVKDVGKFNKTVQEIQVLGSGWDRTLGGDSLNSLIIDDMIAQFVESKGAQKISATAEQVQSHGRAVAKLSKEAERLRHVLSANQNTQASFEGLYEDVDFKYKISRADFETMAKAHVERVNAAIKDALKAANLEIGDLTSVILHGGATRTPFVREAIEKALGSGDKIRTNVNSDEAAVFGAAFRAAELSPSFRVKEIRISEGANYAAGITWKAANGKVHRQRLWTAPSPLGGPAKEITFTEQEDFTGLFYQQVDTEDKPVKSFSTKNLTASVAALKEKYPTCADTGVQFKAAAKLRTENGEVAIVKAFVECEAEVVEKEGFVDGVKNLFGFGKKDQKPLAEGGDKDSADASADSEAETEEASSATKSSSSTSTTKSGDAAESTEAAKEVKKKQLVSIPVEVTLEKAGIPQLTKAEWTKAKDRLKAFAASDKARLQREEALNQLEAFTYKVRDLVDNEAFISASTEAERQTLSEKASEASDWLYEEGDSATKDDFVAKLKALQDLVAPIQNRLDEAEKRPGLISDLRNILNTTNVFIDTVRGQIAAYDEWKSTASAKSAESATSSAAAEATTNDFEGLEDEDDSPKEAEEKPVPEKVVPPLHNSEEIDTLEVLYKETLEWLNKLERQQADVPLTEEPVLVVSELVARRDALDKASLDLALKSYTQYQKNKPKKPTKSKKAKKQDKTKSADKAGPTFEFPEGSVPLSGEELEELVKKYMKEEEETRRQAEGGQAEEKPAEDTEKSSHDEL

SEQ ID NO: 20 - protein, 363 aa, Trichoderma reesei
MVARLSSIYACGLLAWTHIVCASQFSDPMQLQKHLAQNDYTLIAFVASRLEADLKVSLPLTASTSNGREASKLLLEEWQTVQQHVASTATIDCPSSPKLCQEMDVASFPAIRLYRQDGSVTRYRGPRRTAPIDAFVKRALKPSVQNVPGQQLANFITNDDYVFIAKLQGESESINSHYRDFAQEYSDRYSFGIITSGSVPSNGVWCYNNVDGNQHAATDLNDPNALKKLLNLCTAEVIPQLTRRNEMTYLSSGRSLVYYFSNNEADREAYVKALKPIAQRYAEFLQFVTVDSGEYPDMLRNLGVRSAGGLAVQNVHNGHIFPFRGDAAASPGQVDQFIVAISEGRAQPWDGRFDEGQEAHDEL

SEQ ID NO: 21 - protein, 688 aa, Trichoderma reesei
MRLTSFFSGLAAFGLLSSPALADDEADNVPAPTYFDSVMVPPLTELTPDNFEKEASKTKWLLVKHYSPYCHHCISYAPTFQTTYEFYYTSKPEGAGDTSFTDFYDFKFAAVNCIAYSDLCVENGVKLYPTTVLYENGKEVKAVTGGQNITFLSDLIEEALEKSKPGSRPKSLALPQPGDKERPKSEPETASRSATEEKKPKKPVATPNEDGVSVSLTAENFQRLVTMTQDPWFIKFYAPWCPHCQDMAPTWEQLAKNMKGKLNIGEVNCDKESRLCKDVGARAFPTILFFKGGERSEYEGLRGLGDFIKYAENAVDLASGVPDVDLAAFKALEQKEDVIFVYFYDHATTSEDFNALERLPLSLIGHAKLVKTKDPAMYERFKITTWPRFMVSREGRPTYYPPLTPNAMRDTHQVLDWMRSVWLPLVPELLVTNARQIMDNKIVVLGVLNREDQESFQSALREMKSAANEWMDRQIQEFQLERKKLRDAKQMRIEEAEDRDDERALRAAKAIHIDMNNSGRREVAFAWVDGVAWQRWIRTTYGIDVKDGERVIINDQDVSLKLTPICPPSTILLCSRKYWDSTVTGNYILVSRTSILETLDKVVYTPQALKPKLTISSFEKIFFDIRVSFTEHPYLTLGCIVGIAFGAFSWLRGRSRRGRGHFRLEDSISIRDFKDGFLGGSNGNTKAD

SEQ ID NO: 22 - protein, 461 aa, Trichoderma reesei
MHQQTLLATLAASLAALPFAQAGFYSKSSPVLQVDAKSYDRLITKSNHTSIVEFYAPWCGHCQNLKPAYEKAARTLDGLAKVAAVDCDDDANKALCGSLGVKGFPTLKIVRPGKKPGRPVVEDYQGQRTAGAIADAVVAKINNHVVKLTDKDIDAFLEKDGDKPKAILFTEKGTTSALLRSLAIDFLDAVTIGQVRNKEKAAVDRFGISSFPSFVLIPGGGKEPVVYSGELNKKDMVEFLKQVAEPNPDPAPSNGKSGKKASTKDKASSKEAPQKAAAADESSSAASSETSTAAAPESTLIDIPALTSKAELEEHCLQPKSQTCVLAFVPASASEMRNKILSAVSQLHTKYVHGKRHFPFFSVDSDVEGSAALKEALGLSGKIELVALNARRGWWRRYEDGEFSVHSVESWIDAVRMGEGEKKKLPEGVVVEKAEPAEEAKSETEAAAADEATEKPEHDEL

SEQ ID NO: 23 - protein, 368 aa, Trichoderma reesei
MVLIKSLVLAVLASSVAAKSAVIDLIPSNFDKLVFSGKPTLVEFFAPWCGHCKNLAPVYEELAQVFEHAKDKVQIAKVDADSERDLGKRFGIQGFPTLKFFDGKSKEPQEYKSGRDLDSLTKFITEKTGVKPKKKGELPSSVVMLNTRTFHDTVGGDKNVLVAFTAPWCGHCKNLAPTWEKVANDFAGDENVVIAKVDAEGADSKAVAEEYGVTGYPTILFFPAGTKKQVDYQGGRSEGDFVNFINEKAGTFRTEGGELNDIAGTVAPLDTIVANFLSGTGLAEAAAEIKEAVDLLTDAAETKFAEYYVRVFDKLSKNEKFVNKELARLQGILAKGGLAPSKRDEIQIKINVLRKFTPKENEDQKDEL

SEQ ID NO: 24 - protein, 502 aa, Trichoderma reesei
MQQKRLTAALVAALAAVVSAESDVKSLTKDTFNDFINSNDLVLAEFFAPWCGHCKALAPEYEEAATTLKDKSIKLAKVDCVEEADLCKEHGVEGYPTLKVFRGLDKVAPYTGPRKADGITSYMVKQSLPAVSALTKDTLEDFKTADKVVLVAYIAADDKASNETFTALANELRDTYLFGGVNDAAVAEAEGVKFPSIVLYKSFDEGKNVFSEKFDAEAIRNFAQVAATPLVGEVGPETYAGYMSAGIPLAYIFAETAEERENLAKTLKPVAEKYKGKINFATIDAKNFGSHAGNINLKTDKFPAFAIHDIEKNLKFPFDQSKEITEKDIAAFVDGFSSGKIEASIKSEPIPETQEGPVTVVVAHSYKDIVLDDKKDVLIEFYAPWCGHCKALAPKYDELASLYAKSDFKDKVVIAKVDATANDVPDEIQGFPTIKLYPAGDKKNPVTYSGARTVEDFIEFIKENGKYKAGVEIPAEPTEEAEASESKASEEAKASEETHDEL

SEQ ID NO: 25 - protein, 190 aa, Trichoderma reesei
MKAALLLSALASCAIGLVAAAAEDFKIEVTHPVECDRKTQKGDKLSMHYRGTLAKTGDKFDASYDRNQPFNFKLGAGQVIKGWDQGLLDMCIGEKRTLTIPPELGYGQRNMGPIPAGSTLIFETELLAIEGVKAPEKKPVPETPIVEKPAEETEESVVEKAAEAAASVASEAVDAAKTVFADTDEGHGEL

SEQ ID NO: 26 - protein, 207 aa, Trichoderma reesei
MLTFRRLFTTAIVLVVGLLFFVKTAEAAKGPKITHKVFFDIEHGDEKLGRIVLGLYGKTVPETAENFRALATGEKGFGYEGSTFHRVIKQFMIQGGDFTKGDGTGGKSIYGNKFKDENFKLKHTKKGLLSMANAGPDTNGSQFFITTVVTSWLDGRHVVFGEVLEGYDIVEKIENVQTGPGDRPVKPVKIAKSGELEVPPEGIHVEL

SEQ ID NO: 27 - protein, 413 aa, Trichoderma reesei
MILRAAIFVLLALVSLAVCAEDFYKVLGVDKSASDKQLKQAYRQLSKKFHPDKNPGDETAHEKFVLVSEAYEVLSDSELRKVYDRYGHEGVKSHRQGGGGGGGGDPFDLFSRFFGGHGHFGRNSREPRGSNIEVRIEISLRDFYNGATTEFQWEKQHICEKCEGTGSADGKVETCSVCGGHGVRIVKQQLVPGMFQQMQMRCDHCGGSGKTIKNKCSVCHGSRVERKPTTVSLTVERGIARDAKVVFENEADQSPDWVPGDLIVNLGEKAPSYEDNPDRVDGTFFRRKGHDLYWTEVLSLREAWMGGWTRNLTHLDKHVVRLGRERGQVVQSGLVETIPGEGMPIWHEEGESVYHTHEFGNLYVTYEVILPDQMDKKMESEFWDLWEKWRSKNGVDLQKDLGRPEPGHDHDEL

SEQ ID NO: 28 - protein, 182 aa, Trichoderma reesei
MARRQHLTATVLLAVVLFFSITYLLSGSSSSNADRTREAVVAEPKSEFKVDFDGMPANLLEGESIAPKLENATLKAELGRATWKFMHTMVARFPEKPSPEERKTLETFIYLFGRLYPCGDCARHFRGLLAKYPPQTSSRNAAAGWLCFVHNQVNERLKKPIFDCNNIGDFYDCGCGDEKKDG

SEQ ID NO: 29 - protein, 1070 aa, Trichoderma reesei
MVMLVAIALAWLGCSLLRPVDAMRADYLAQLRQETVDMFYHGYSNYMEHAFPEDELRPISCTPLTRDRDNPGRISLNDALGNYSLTLIDSLSTLAILAGGPQNGPYTGPQALSDFQDGVAEFVRHYGDGRSGPSGAGIRARGFDLDSKVQVFETVIRGVGGLLSAHLFAIGELPITGYVPRPEGVAGDDPLELAPIPWPNGFRYDGQLLRLALDLSERLLPAFYTPTGIPYPRVNLRSGIPFYVNSPLHQNLGEAVEEQSGRPEITETCSAGAGSLVLEFTVLSRLTGDARFEQAAKRAFWEVWHRRSEIGLIGNGIDAERGLWIGPHAGIGAGMDSFFEYALKSHILLSGLGMPNASTSRRQSTTSWLDPNSLHPPLPPEMHTSDAFLQAWHQAHASVKRYLYTDRSHFPYYSNNHRATGQPYAMWIDSLGAFYPGLLALAGEVEEAIEANLVYTALWTRYSALPERWSVREGNVEAGIGWWPGRPEFIESTYHIYRATRDPWYLHVGEMVLRDIRRRCYAECGWAGLQDVQTGEKQDRMESFFLGETAKYMYLLFDPDHPLNKLDAAYVFTTEGHPLIIPKSKRGSGSHNRQDRARKAKKSRDVAVYTYYDESFTNSCPAPRPPSEHHLIGSATAARPDLFSVSRFTDLYRTPNVHGPLEKVEMRDKKKGRVVRYRATSNHTIFPWTLPPAMLPENGTCAAPPERIISLIEFPANDITSGITSRFGNHLSWQTHLGPTVNILEGLRLQLEQVSDPATGEDKWRITHIGNTQLGRHETVFFHAEHVRHLKDEVFSCRRRRDAVEIELLVDKPSDTNNNNTLASSDDDVVVDAKAEEQDGMLADDDGDTLNAETLSSNSLFQSLLRAVSSVFEPVYTAIPESDPSAGTAKVYSFDAYTSTGPGAYPMPSISDTPIPGNPFYNFRNPASNFPWSTVFLAGQACEGPLPASAPREHQVIVMLRGGCSFSRKLDNIPSFSPHDRALQLVVVLDEPPPPPPPPPANDRRDVTRPLLDTEQTTPKGMKRLHGIPMVLVRAARGDYELFGHAIGVGMRRKYRVESQGLVVENAVVL

SEQ ID NO: 30 - protein, 406 aa, Trichoderma reesei
MRPLALIFALILGLLLCLAAPATASSSSSQHSPQAASDESDLICHTSNPDECYPRVFVPTHEFQPVHDDQQLPNGLHVRLNIWTGQKEAKINVPDEANPDLDGLPVDQAVVLVDQEQPEIIQIPKGAPKYDNVGKIKEPAQEGDAQTEAIAFAETFNMLKTGKSPSAEEFDNGLEGLEELSHDIYYGLKITEDADVVKALFCLMGARDGDASEGATPRDQQAAAILAGALSNNPSALAEIAKIWPELLDSSCPRDGATISDRFYQDTVSVADSPAKVKAAVSAINGLIKDGAIRKQFLENSGMKQLLSVLCQEKPEWAGAQRKVAQLVLDTFLDEDMGAQLGQWPRGKASNNGVCAAPETALDDGCWDYHADRMVKLHGTPWSKELKQRLGDARKANSKLPDHGEL

SEQ ID NO: 31 - DNA, 51 bp, synthetic construct
  misc_feature 1..51: note = F-attB1 PCR primer
  source 1..51: mol_type = other DNA
ggggacaagtttgtacaaaaaagcaggctatggctcgttcacggagctccc

SEQ ID NO: 32 - DNA, 53 bp, synthetic construct
  misc_feature 1..53: note = R-attB2 PCR prim
  source 1..53: mol_type = other DNA
ggggaccactttgtacaagaaagctgggtttacaattcgtcgtggaagtcgcc

SEQ ID NO: 33 - DNA, 21 bp, Trichoderma reesei
  source 1..21: mol_type = other DNA
agttaccacgagcggtaacag

SEQ ID NO: 34 - DNA, 19 bp, Trichoderma reesei
  source 1..19: mol_type = other DNA
aagagaactcgttgccaag

SEQ ID NO: 35 - DNA, 20 bp, Trichoderma reesei
  source 1..20: mol_type = other DNA
caccaacaccgtctacgatg

SEQ ID NO: 36 - DNA, 23 bp, Trichoderma reesei
  source 1..23: mol_type = other DNA
cgttcttctcaatgaccttgtag

SEQ ID NO: 37 - DNA, 16 bp, synthetic construct
  misc_feature 1..16: note = Chymosin forward
  source 1..16: mol_type = other DNA
cagcaagctcgtcggc

SEQ ID NO: 38 - DNA, 22 bp, synthetic construct
  misc_feature 1..22: note = Chymosin reverse
  source 1..22: mol_type = other DNA
ggtacatcttgccgttgatctc

SEQ ID NO: 39 - DNA, 22 bp, synthetic construct
  misc_feature 1..22: note = CBHI linker region with SpeI restriction site change
  source 1..22: mol_type = other DNA
ggtacatcttgccgttgatctc

SEQ ID NO: 40 - DNA, 23 bp, synthetic construct
  misc_feature 1..23: note = hph1 PCR primer
  source 1..23: mol_type = other DNA
tctccggtgtcccttgtcccttc

SEQ ID NO: 41 - DNA, 23 bp, synthetic construct
  misc_feature 1..23: note = hph2 PCR primer
  source 1..23: mol_type = other DNA
acctgtggcgccggtgatgccgg

SEQ ID NO: 42 - DNA, 1113 bp, synthetic construct
  misc_feature 1..1113: note = synthetic pro-chymosin gene
  source 1..1113: mol_type = other DNA
actagtgccgagatcacccgcatccctctctacaagggcaagagcctccgcaaggccctcaaggagcacggcctcctcgaggacttcctccagaagcagcagtacggcatcagcagcaagtacagcggcttcggcgaggtcgccagcgtccctctcaccaactacctcgacagccagtacttcggcaagatctacctcggcacccctcctcaggagttcaccgtcctcttcgacaccggcagcagcgacttctgggtcccgagcatctactgcaagagcaacgcctgcaagaaccaccagcgcttcgaccctcgcaagagcagcaccttccagaacctcggcaagcctctcagcatccactacggcaccggcagcatgcagggcatcctcggctacgacaccgtcacggtctccaacatcgtcgacatccagcagactgtcggcctcagcacccaggagcctggcgacgtctttacctacgccgagttcgacggcatcctgggcatggcctaccctagcctcgccagcgagtacagcatccctgtctttgacaacatgatgaaccgccacctcgtcgcccaggacctcttcagcgtctacatggaccgcaacggccaagagagcatgctcaccctcggcgccatcgaccctagctactacaccggcagcctccactgggtcccggtcaccgtccagcagtactggcagttcaccgtcgacagcgtcaccatcagcggcgtcgtcgtcgcctgcgagggcggctgccaggccatcctggacaccggcaccagcaagctcgtcggccctagcagcgacatcctgaacatccagcaggccatcggcgccacccagaaccagtacggcgagttcgacatcgactgcgacaacctcagctacatgcctaccgtcgtctttgagatcaacggcaagatgtaccctctcacccctagcgcctacaccagccaggaccagggcttctgcaccagcggcttccagagcgagaaccacagccagaagtggatcctcggcgacgtctttatccgcgagtactacagcgtctttgaccgcgccaacaacctcgtcggcctcgccaaggccatctaaggcgcgccg

SEQ ID NO: 43 - DNA, 1401 bp, Trichoderma reesei
  source 1..1401: mol_type = other DNA
ccacgaggtccaatatcgctatgcttgtgaggctgccatgggtctgagttcgtattgccgaatgaggcaagtgatttatctatacacgtacatacagcactagagaatgaatcatgactccgcttcgtatgtaatacttgtagcttggccggctggtcgggacacatcatggcaatgcgacgcctcaatggctacgtgtgcttttaccacaacaaagatagatgcactgattctacttgtttctctcaagaccggccgtgctggcagagacagtaaggcaagaaattgagtggcatcttgagcgcttcagccttgccgtggcaaagcgccgatacgtggcactttgccgcttgaaccaaccgatatccacccggataccggttaaggtgcctattgacagcctggaagtacctgtgagacaatgacggctaaggacatcaactacaattgtaagcataatccgctcgggcaaatattagaaaacgccaacgtgtccataacggtcgtgggagagaccagcttccttttccgtttgccacaagtcatcgatacggatacagcttcatatggtacgagtacttacgtgtacttgacagggactccgtcaacagcaaagactcaaggcgaatctgctcctgatgaagaggtagctccgggtcacgttccgggctggaatagcatcgtaacttccaccacgttccgtcacccgcccagcggcaaacgggcaaattttagtgcggcgcagaagaagaggccacttctcgcttctccacgcttcttgtgctccctgggcctggaggggcaaaacggggacgaatcgtggctgttgtaagcggctccgggtgaacgagttaggtaggtgtctctgttcctagcgggcgactccatggctgcacctttgacggctgtgcttgctcggtagttgatcgcactgtattttgcgcaaatttccagtgatttgagacagtgctaaccgtgcgagatcgcccgccgcaagacccctgtcacgtggggctgcgtgttccggggccttccactgcctgtccctcttctagcgtttagcgacccgttgcctccaacagacctgaaatctggcctttttgagccttctggacacctcaactttttctggggcgcttcataaagtggcccatcgtcacctctcggcttcaactcgagtttttccctttttccttttcttcttcttcttcaaacaaagataccccccctcaacccggtgcaaccagcctggtccgaggacaaaacacgatagagctcgctgcgttggatcgctgcgcgtcttctcttgttctctctctcttttcttctctgcaacgcttataactctttttgcgcggggcatctgggaaaaccgtttcttcacacatctcttcttccaca

SEQ ID NO: 44 - DNA, 1095 bp, Bos taurus
  source 1..1095: mol_type = other DNA
gccgagattactcgaatccccctctacaagggcaagagcctccgaaaggccctcaaggagcacggcctcctcgaggacttcctccagaagcagcagtacggcatcagcagcaagtacagcggcttcggcgaggtcgccagcgtccctctcaccaactacctcgacagccagtacttcggcaagatctacctcggcacccctcctcaggagttcaccgtcctcttcgacaccggcagcagcgacttctgggtccccagcatctactgcaagagcaacgcctgcaagaaccaccagcgcttcgacccccgcaagagcagcaccttccagaacctcggcaagcctctcagcatccactacggcaccggcagcatgcagggcatcctcggctacgacaccgtcaccgtcagcaacatcgtcgacatccagcagaccgtcggcctcagcacccaggagcctggcgacgtcttcacttacgccgagttcgacggtatcctcggcatggcctaccccagcctcgccagcgagtacagcatccccgtcttcgacaacatgatgaaccgccacctcgtcgcccaggacctcttcagcgtctacatggaccgaaacggccaggagagcatgctcaccctcggcgccatcgaccctagctactacactggcagcctccactgggtccctgtcaccgtccagcagtactggcagttcaccgtcgacagcgtcaccatcagcggcgttgtcgtcgcttgcgagggtggttgccaggctatcctcgacaccggcactagcaagctcgtcggccctagcagcgacatcctcaacatccagcaggccatcggcgccacccagaaccagtacggcgagttcgacatcgactgcgacaacctcagctacatgcccaccgtcgtcttcgagatcaacggcaagatgtaccctctcacccctagcgcctacactagccaggaccagggcttctgcaccagcggcttccagagcgagaaccacagccagaagtggatcctcggcgacgtcttcatccgcgagtactacagcgtcttcgaccgcgccaacaacctcgtcggcctcgccaaggccatc

SEQ ID NO: 45 - DNA, 1095 bp, Camelus dromedarius
  source 1..1095: mol_type = other DNA
agcggcattactcgaatccccctccacaagggcaagaccctccgaaaggctctcaaggagcgaggcctcctcgaggacttcctccagcgacagcagtacgccgtcagcagcaagtacagcagcctcggcaaggtcgcccgagagcctctcaccagctacctcgacagccagtacttcggcaagatctacatcggcacccctcctcaggagttcaccgtcgtcttcgacactggcagcagcgacctctgggtccccagcatctactgcaagagcaacgtctgcaagaaccaccaccgcttcgacccccgaaagagcagcaccttccgcaacctcggcaagcctctcagcatccactacggcaccggcagcatggagggcttcctcggctacgacaccgtcaccgtcagcaacatcgtcgaccccaaccagactgtcggcctcagcaccgagcagcctggcgaggtcttcacctacagcgagttcgacggcattctcggcctcgcctaccctagcctcgccagcgagtacagcgtccctgtcttcgacaacatgatggaccgacacctcgtcgcccgagacctcttcagcgtctacatggaccgaaacggccagggcagcatgctcaccctcggcgccattgaccctagctactacactggcagcctccactgggtccccgtcaccctccagcagtactggcagttcactgtcgacagcgtcaccatcaacggcgtcgctgtcgcttgcgtcggtggctgccaggctattctcgacactggcactagcgtcctcttcggccccagcagcgacatcctcaagatccagatggccatcggcgccaccgagaaccgatacggcgagttcgacgtcaactgcggcaacctccgaagcatgcccaccgtcgtcttcgagatcaacggccgagactaccccctcagccctagcgcctacactagcaaggaccagggcttctgcaccagcggcttccagggcgacaacaacagcgagctctggatcctcggcgacgtcttcatccgcgagtactacagcgtcttcgaccgcgccaacaaccgcgtcggcctcgccaaggccatc

SEQ ID NO: 46 - DNA, 1095 bp, Lama glama
  source 1..1095: mol_type = other DNA
agcggcatcacccgcatccccctctacaagggcaagaccctccgcaaggctctcaaggagcacggcctcctcgaggacttcctccagcgacagcagtacgccgtcagcagcaagtacagcagcctcggcaaggtcgcccgagagcctctcaccagctacctcgacagccagtacttcggcaagatctacatcggcacccctcctcaggagttcactgtcgtcttcgacaccggcagcagcgacctctgggtccccagcatctactgcaagagcaacgtctgcaagaaccaccaccgcttcgacccccgcaagagcagcaccttccgaaacctcggcaagcctctcagcatccactacggcaccggcagcatggagggcttcctcggctacgacaccgtcaccgtcagcaacatcgtcgaccccaaccagaccgtcggcctcagcactgagcagcctggcgaggtcttcacctacagcgagttcgacggcaacctcggcctcgcctaccctagcctcgctagcgagtacagcgtccctgtcttcgacaacatgatggaccgacacctcgtcgcccaggacctcttcagcgtctacatggaccgaaacggccagggcagcatgctcaccctcggcgccatcgacagcagctactacactggtagcctccactgggtccccgtcaccgtccagcagtactggcaggtcactgtcgacagcgtcaccatcaacggcgtcgctgtcgcttgcgtcggtggctgccaggctattctcgacactggcactagcgtcctcttcggccccagcagcgacatcctcaagatccagaaggccatcggcgccaccgagaaccgatacggcgagttcgacgtcaactgcggcaacctccgaagcatgcccaccgtcgtcttcgagatcaacggccgagactaccccctcagccctagcgcctacactagcaaggaccagggcttctgcaccagcggcttccagggcgacaacaacagcgagctctggatcctcggcgacgtcttcatccgcgagtactacagcgtcttcgaccgcgccaacaaccgcgtcggcctcgccaaggccatc

SEQ ID NO: 47 - DNA, 1112 bp, Vicugna pacos
  source 1..1112: mol_type = other DNA
agccccctcctccgaatccccctccacaagggcaagaccctccgaaaggctctcaaggagcacggcctcctcgaggacttcctccagcgacagcagtacgccgtcagcagcaagtacagcagcctcggcaaggtcgcccgagagcctctcaccagctacctcgacagccagtacttcggcaagatctacatcggcacccctcctcaggagttcactgtcgtcttcgacaccggcagcagcgacctctgggtccccagcatctactgccgcagcaacgtctgcaagaaccaccaccgcttcgacccccgaaagagcagcaccttccgcaacctcggcaagcctctcagcatccactacggcaccggcagcatggagggcttcctcggctacgacaccgtcaccgtcagcaacatcgtcgaccccaaccagactgtcggcctcagcaccgagcagcctggcgaggtcttcacctacagcgagttcgacggcattctcggcctcgcctaccctagcctcgccagcgagtacagcgtccctgtcttcgacaacatgatggaccgacacctcgtcgcccaggacctcttcagcgtctacatggaccgaaacggccagggcagcatgctcaccctcggcgccattgaccctagctactacactggcagcctccactgggtccccgtcaccgtccagcagtactggcagttcactgtcgacagcgtcaccatcaacggcgtcgctgtcgcttgcgtcggtggctgccaggctattctcgacactggcactagcgtcctcttcggccccagcagcgacatcctcaagatccagaaggccatcggcgccactgagaaccgatacggcgagttcgacgtcaactgcggcagcctccgaagcatgcccaccgtcgtcttcgagatcaacggccgagactaccccctcagccctagcgcctacactagcaaggaccagggcttctgcaccagcggcttccagggcgacaacaacagcgagctctggatcctcggcgacgtcttcatccgcgagtactacagcgtcttcgaccgcgccaacaaccgcgtcggcctcgccaaggccatcgcctcgccaaggccatc

SEQ ID NO: 48 - protein, 365 aa, Bos taurus
AEITRIPLYKGKSLRKALKEHGLLEDFLQKQQYGISSKYSGFGEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSDILNIQQAIGATQNQYGEFDIDCDNLSYMPTVVFEINGKMYPLTPSAYTSQDQGFCTSGFQSENHSQKWILGDVFIREYYSVFDRANNLVGLAKAI

SEQ ID NO: 49 - protein, 365 aa, Camelus dromedarius
SGITRIPLHKGKTLRKALKERGLLEDFLQRQQYAVSSKYSSLGKVAREPLTSYLDSQYFGKIYIGTPPQEFTVVFDTGSSDLWVPSIYCKSNVCKNHHRFDPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVLFGPSSDILKIQMAIGATENRYGEFDVNCGNLRSMPTVVFEINGRDYPLSPSAYTSKDQGFCTSGFQGDNNSELWILGDVFIREYYSVFDRANNRVGLAKAI

SEQ ID NO: 50 - protein, 365 aa, Lama glama
SGITRIPLYKGKTLRKALKEHGLLEDFLQRQQYAVSSKYSSLGKVAREPLTSYLDSQYFGKIYIGTPPQEFTVVFDTGSSDLWVPSIYCKSNVCKNHHRFDPRKSSTFRNLGKPLSIHYGTGSMEGFLGYDTVTVSNIVDPNQTVGLSTEQPGEVFTYSEFDGNLGLAYPSLASEYSVPVFDNMMDRHLVAQDLFSVYMDRNGQGSMLTLGAIDSSYYTGSLHWVPVTVQQYWQVTVDSVTINGVAVACVGGCQAILDTGTSVLFGPSSDILKIQKAIGATENRYGEFDVNCGNLRSMPTVVFEINGRDYPLSPSAYTSKDQGFCTSGFQGDNNSELWILGDVFIREYYSVFDRANNRVGLAKAI

SEQ ID NO: 51 - protein, 365 aa, Vicugna pacos
SPLLRIPLHKGKTLRKALKEHGLLEDFLQRQQYAVSSKYSSLGKVAREPLTSYLDSQYFGKIYIGTPPQEFTVVFDTGSSDLWVPSIYCRSNVCKNHHRFDPRKSSTFRNLGKPLSIHYGTGSMEGFLGYDTVTVSNIVDPNQTVGLSTEQPGEVFTYSEFDGILGLAYPSLASEYSVPVFDNMMDRHLVAQDLFSVYMDRNGQGSMLTLGAIDPSYYTGSLHWVPVTVQQYWQFTVDSVTINGVAVACVGGCQAILDTGTSVLFGPSSDILKIQKAIGATENRYGEFDVNCGSLRSMPTVVFEINGRDYPLSPSAYTSKDQGFCTSGFQGDNNSELWILGDVFIREYYSVFDRANNRVGLAKAI

SEQ ID NO: 52 - protein, 514 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 53 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 54 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 55 - protein, 418 aa, Trichoderma reesei
MNKSVAPLLLAASILYGGAAAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 56 - protein, 234 aa, Trichoderma reesei
MKFLQVLPALIPAALAQTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 57 - protein, 344 aa, Trichoderma reesei
MIQKLSNLLVTALAVATGVVGHGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 58 - protein, 242 aa, Trichoderma reesei
MKATLVLGSLIVGAVSAYKATTTRYYDGQEGACGCGSSSGAFPWQLGIGNGVYTAAGSQALFDTAGASWCGAGCGKCYQLTSTGQAPCSSCGTGGAAGQSIIVMVTNLCPNNGNAQWCPVVGGTNQYGYSYHFDIMAQNEIFGDNVVVDFEPIACPGQAASDWGTCLCVGQQETDPTPVLGNDTGSTPPGSSPPATSSSPPSGGGQQTLYGQCGGAGWTGPTTCQAPGTCKVQNQWYSQCLP

SEQ ID NO: 59 - protein, 838 aa, Trichoderma reesei
MKVSRVLALVLGAVIPAHAAFSWKNVKLGGGGGFVPGIIFHPKTKGVAYARTDIGGLYRLNADDSWTAVTDGIADNAGWHNWGIDAVALDPQDDQKVYAAVGMYTNSWDPSNGAIIRSSDRGATWSFTNLPFKVGGNMPGRGAGERLAVDPANSNIIYFGARSGNGLWKSTDGGVTFSKVSSFTATGTYIPDPSDSNGYNSDKQGLMWVTFDSTSSTTGGATSRIFVGTADNITASVYVSTNAGSTWSAVPGQPGKYFPHKAKLQPAEKALYLTYSDGTGPYDGTLGSVWRYDIAGGTWKDITPVSGSDLYFGFGGLGLDLQKPGTLVVASLNSWWPDAQLFRSTDSGTTWSPIWAWASYPTETYYYSISTPKAPWIKNNFIDVTSESPSDGLIKRLGWMIESLEIDPTDSNHWLYGTGMTIFGGHDLTNWDTRHNVSIQSLADGIEEFSVQDLASAPGGSELLAAVGDDNGFTFASRNDLGTSPQTVWATPTWATSTSVDYAGNSVKSVVRVGNTAGTQQVAISSDGGATWSIDYAADTSMNGGTVAYSADGDTILWSTASSGVQRSQFQGSFASVSSLPAGAVIASDKKTNSVFYAGSGSTFYVSKDTGSSFTRGPKLGSAGTIRDIAAHPTTAGTLYVSTDVGIFRSTDSGTTFGQVSTALTNTYQIALGVGSGSNWNLYAFGTGPSGARLYASGDSGASWTDIQGSQGFGSIDSTKVAGSGSTAGQVYVGTNGRGVFYAQGTVGGGTGGTSSSTKQSSSSTSSASSSTTLRSSVVSTTRASTVTSSRTSSAAGPTGSGVAGHYAQCGGIGWTGPTQCVAPYVCQKQNDYYYQCV

SEQ ID NO: 60 - protein, 437 aa, Trichoderma reesei
MMMLSKSLLSAATAASALAAVLQPVPRASSFVTISGTQFNIDGKVGYFAGTNCYWCSFLTNHADVDSTFSHISSSGLKVVRVWGFNDVNTQPSPGQIWFQKLSATGSTINTGADGLQTLDYVVQSAEQHNLKLIIPFVNNWSDYGGINAYVNAFGGNATTWYTNTAAQTQYRKYVQAVVSRYANSTAIFAWELGNEPRCNGCSTDVIVQWATSVSQYVKSLDSNHLVTLGDEGLGLSTGDGAYPYTYGEGTDFAKNVQIKSLDFGTFHLYPDSWGTNYTWGNGWIQTHAAACLAAGKPCVFEEYGAQQNPCTNEAPWQTTSLTTRGMGGDMFWQWGDTFANGAQSNSDPYTVWYNSSNWQCLVKNHVDAINGGTTTPPPVSSTTTTSSRTSSTPPPPGGSCSPLYGQCGGSGYTGPTCCAQGTCIYSNYWYSQCLNT

SEQ ID NO: 61 - protein, 222 aa, Trichoderma reesei
MVSFTSLLAASPPSRASCRPAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 62 - protein, 347 aa, Trichoderma reesei
MKANVILCLLAPLVAALPTETIHLDPELAALRANLTERTADLWDRQASQSIDQLIKRKGKLYFGTATDRGLLQREKNAAIIQADLGQVTPENSMKWQSLENNQGQLNWGDADYLVNFAQQNGKSIRGHTLIWHSQLPAWVNNINNADTLRQVIRTHVSTVVGRYKGKIRAWDVVNEIFNEDGTLRSSVFSRLLGEEFVSIAFRAARDADPSARLYINDYNLDRANYGKVNGLKTYVSKWISQGVPIDGIGSQSHLSGGGGSGTLGALQQLATVPVTELAITELDIQGAPTTDYTQVVQACLSVSKCVGITVWGISDKDSWRASTNPLLFDANFNPKPAYNSIVGILQ

SEQ ID NO: 63 - protein, 744 aa, Trichoderma reesei
MRYRTAAALALATGPFARADSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 64 - protein, 612 aa, Trichoderma reesei
MAKLSTLRLASLLSLVSVQVSASVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 65 - protein, 256 aa, Trichoderma reesei
MFIAGVALSALLCADTVLAGVAQDRGLAARLARRAGRRSAPFRNDTSHATVQSNWGGAILEGSGFTAASATVNVPRGGGGSNAAGSAWVGIDGASCQTAILQTGFDWYGDGTYDAWYEWYPEFAADFSGIDIRQGDQIAMSVVATSLTGGSATLENLSTGQKVTQNFNRVTAGSLCETSAEFIIEDFEECNSNGSNCQPVPFASFSPAITFSSATATRSGRSVSLSGAEITEVIVNNQDLTRCSVSGSSTLTCSYV

SEQ ID NO: 66 - protein, 637 aa, Trichoderma reesei
MKIGLIVLNSLLAGAIAVPAGLQIVHEKRDTSNARWTKREALNPNTKVPVRIALKQNNLDKGMDYLLDVSHPKSANYGNHYTADQVVDLFAPSPPSVAAVTAWLVKSGIPASSITASKSKGWLDFTTTSGQLESLLQTSYNTYQHVEARNVHVGTDEYRLPEEISQHIDFITPGVVFSPMKKSSGAEKRGEKGIRRASKPIPADIAQILRENPLATSTCGSAITPQCIRSMYNITAGTTAVSTNALGIFETGDTYAQQDLTLFWENFATNIPSSTGPKVDEIDGATAPTSPFNAGGESDLDFEIAIPIIYPQTTVLYQAAVKNDDIFNTFLDAIDGSYCTFSDDGETGDDPTIDGTTRNEQCGSFKPTNVISFSYGTAEADYPTYYLQRQCDEFMKLGLQGKSIVFASGDDGVARRSGPCLGPKRNIFTPGEPASCPYVTSVGATTLPSGKQPGDAETAVTTFSSGGGFSNIWTTPSYQADAIASYFASHDPGYASYNTSGGVIPTTGGIYNRAGRGYPDVSAVGDNGVVVNKEEEVLEGGTSMSAPLFAGILTRINEERIAAGKSPIGFANPALYANPSMFHDITVGNQAKGGPNGDSGASACGNTGFSAVSGWDPVTGLGTPDYPALLEYFLGLA

SEQ ID NO: 67 - protein, 401 aa, Trichoderma reesei
MTILSRLAVGVALLAFPELIDAHRVPVKRPNLQWGPKSPGHAFPHSPKRHKTCYVPSCGSNDAPEILKAFKRCNRGGTVVLNEEYVIASPLDLTFLESVDVAITGTIKFTNDIDFWVVNSFKYDFQNSSAFWRFGGKDVNIYGGGHGLIDGNGQAWIEDAKFARCRYLFAYSSGIVANVVQWFNLIANSSDILVSDIDIAVKSESKNPAKNGDGWDTFRSDSVVIQDSYVNNSDDCVSFKPNSTNIIVQGMQCNGSHGISVGSLGQYPAEYDIVEHVYVYNISMSNASDGARIKVWPGTDTPFEPGLSGGGGAGYVKNVTYDTFHNNNNDWAIEINQCYGQSNQTICDKYPSNMTISDVVFKNMWGTTSKKYDPKVGTLTCSSTESSLEKREYRGEEHLGG

SEQ ID NO: 68 - protein, 379 aa, Trichoderma reesei
MLKLSLFLGAVTASLCVQAHAVPPPTVTQAPKLEDRATTCTFSGSNGASSASKSQKSCATIVLSNVAVPSGVTLDLSDLNDGTTVIFEGTTTWGYKEWSGPLLQIEGNDITIQGASGAVLNPDGARWWDGQGGNGGKTKPKFFAAHDLTSSSITNLYIKNTPVQAVSVNGVNGLTITGMTIDNSAGDSGGGHNTDGFDIGSSSNVVISGAKVYNQDDCVAVNSGTNITFTGGLCSGGHGLSIGSVGGRDDNTVQTVTFSNSQVTKSANGIRIKATAGKTGTIKGVTYTGITLSSITGYGILIEQNYDGGDLHGSPTSGIPITNLVLQNISGSNGVVSSGNNIAIVCGSGACSNWTWSNVVVTGGKKYGSCQNVPSPATC

SEQ ID NO: 69 - protein, 463 aa, Trichoderma reesei
MKLRYALPLLLQLSLPVLSADTAAWRSRTIYFALTDRIARGSGDTGGSACGNLGDYCGGTFQGLESKLDYIKGMGFDAIWITPVVTNSDGGYHGYWAEDIDSINSHYGSADDLKSLVNAAHSKGFYMMVDVVANHMGYANISDDSPSPLNQASSYHPECDIDYNNQTSVENCWISGLPDLNTQSSTIRSLYQDWVSNLVSTYGFDGVRIDTVKHVEQDYWPGFVNATGVYCIGEVFDGDPNYLLPYASLMPGLLNYAIYYPMTRFFLQQGSSQDMVNMHDQIGSMFPDPTALGTFVDNHDNPRFLSIKNDTALLKNALTYTILSRGIPIVYYGTEQAFSGGNDPANREDLWRSGFNAQSDMYDAISKLTYAKHAVGGLADNDHKHLYVADTAYAFSRAGGNMVALTTNSGSGSSAQHCFGTQVPNGRWQNVFDEGNGPTYSADGNGQLCLNVSNGQPIVLLSS

SEQ ID NO: 70 - protein, 691 aa, Trichoderma reesei
MAASQDEQKIPQDGTGVIQLDPWLAPFSDALKRRYSKAQDWIKRINDTEGGFEKFSKGTEIFGFNVDEKNNVTYREWAPNAEQAYLVGDFNGWNYESHPMKKNAYGVFEIVVPAKGKEKAIPHNSKVKISLVLPGGSRVDRLPAWAKYVTQDLSVSPAYDARFWSPEPSDKYAFKHPRPQKPQSIRIYEAHVGISSPEQRVTTYDEFTDNVLPRIKDLGYNAIQLMAIMEHAYYASFGYQVNSFFAASSRYGTPEGLKRLVDTAHQMGIVVLLDVVHSHASKNVLDGLNQFDGTDHQYFHSGGKGNHDLWDSRLFNYGHHEVMRFLLSNLRFWMDEYHFDGFRFDGVTSMLYKHHGIGTGFSGGYHEYFGFDVDEEAVVYLMVANEMLHSLYPECVTVAEDVSGMPALCLPLSLGGIGFDYRLAMAIPDMWIKILKEKKDEDWDLANICFTLTNRRHGEKTIAYCESHDQALVGDKTLMMHLCDAELYTNMSTLSPLTPVIDRGMALHKMIRLLTHGLGGEGYLNFEGNEFGHPEWLDFPRAGNNNSFWYARRQFNLTEDPLLRYKFLNNFDKLMNHCEAKYGWLHAPQAYISLKHEGDKVIVFERAGLVFVFNFHTHRSFSDYRIGIEVPGTYKIVLNSDSEEVGGHNRVDEETRFFTTPMEWNGRKNWTHIYIPCRTALVLALESPATQ

SEQ ID NO: 71 - protein, 632 aa, Trichoderma reesei
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 72 - protein, 1557 aa, Trichoderma reesei
MSPRIMTTTEVYLLPLNDDGSPQVAGEYIYLEPKSDDPVTVRFAIEGTSSVCRNGSLWVNIPEQGREFRRDKFREYKLTPDFNRTLEISIPIYESGVFAFYTTYAELPKLDDDDVPKPSDRHEALKKTPVYYIDVAPRLKLDGRPLPLPALSLFSIISKLMGRFPGDWERHLRGISERGYNMIHFTPLQVRGASNSPYSLYDQLGWDPACFPRGEKDIEDLVESLEQKYSLLSLTDIVLNHTAHNTKWLQEHPEAGYNLVTAPWLQSAYELDTKLLELGGQLEKLGLPVDIKTTEDLLQLMEGIKEHVILPLQLWEYYAINVERDADRIVEAWVGGKAIPPQDGSATLNDFEHLKTAELPQQVEFVKKYALQGTDRLGERFRRRVEPNVAANLLSFAFGRYEGESADTVNTTAAKAKLVEILDAINVPFYEEYDKDLNQILEQLFNRIKYVRMDDHGPKLGPINAANPIIETYFSRLPENETTAKLSPGERALANNGWVWGGNALVDNAGPDSRVYLRREVIVWGDCVKLRYGSRREDSPWLWDHMTEYARMLAKHFAGFRIDNCHSTPIHVAEHILDEARRVRPNLYVVAELFTGSEEMDYVFVKRLGISSLIREAMQAWSTGELSRLVHRHGGRPIGSFEVDDISKSDVNTPSGSKGKSDLTREIIRTIRPSPVHALFMDCTHDNEPPAQKRDARDTLPNAALVNMCSSATGSVMGYDEIYPKLVDLVNEKRLYTSESSRTPTIKIGRGKGGIGSIKKLLNQIHTLMGKDGYNETHIHHEGEYITVHRVHPESRKGYFLIAHTAFPGYGNGRGEFNPVLLTGTRANHLGSWMLEVDDREETVKTVQSDTKFLRGLPSQVVDVPGIKMEYHGQDTTISVRDKFPPGSIALFETWIPTAEHSTGLDNYVTSGAKAAWANLNLIDLNFLLYRCEAEERDATDGQDGVYDIPGHGKLVYAGLQGWWSVLEGVIRDNNLGHPVCQNLRDGHWALDYIVGRIQKQSHVFGSEGLSGPAAWLRERFEAIRAIPSFLLPRYFALAIRTAYRASCDRAFELMNEHVAKGQWFLGSLALVSIQQTGLVKSASLWPDKQVPSIAAGLPHFAVQWARCWGRDVFISIRGLYLGTSRFAEAKEHILAFASVLKHGMIPNLLSSGAAPRYNSRDSVWFFLQAIQDYTRFAPEGLDLLKVKVRRRFLPYDDTWFPVDDERTYSVESSIEEIIQEALQRHASGMKFREANAGPQIDSQMRDEGFNQEIKVDWSNGIVFGGNQFNCGTWMDKMGESEKAGSKGIPGTPRDGAAIEITGLLYSTLRWVEQLSKQGKFAYPGVKTAEDKTVSWTQWADLIKANFERCYYVPLSPEDDAKYDVNPSVVARRGIYKDLYKSGKEYEDYQLRPNFAIAMTVASDLFDPKHAWHALCLADSTIRGPTGMATLDPADKDYRPYYRNSEDSHDFATSKGRNYHQGPEWLWPTGFFLRALLKFDLQRRKTAEGRTEAFQQVTRRLAGCKRAIRESPWAGLTELTQKNGEFCPDSSPTQAWSAGCLIDLYMDAMEVQDGFRN